(19) United States
(12) Patent Application Publication
(10) Pub. No.: US 2026/0174678 A1
(43) Pub. Date: Jun. 25, 2026

(54) OLIGOSACCHARIDES FOR GLYCEMIC CONTROL

(71) Applicant: ONE BIO INC, Sacramento, CA (US)

(72) Inventors: Matthew Joseph AMICUCCI, Sacramento, CA (US); Angela Maria MARCOBAL-BARRANCO, Sacramento, CA (US); Maria Ximena MALDONADO-GOMEZ, Sacramento, CA (US); Cory Glen VIERRA, Sacramento, CA (US); James Patrick CERNEY, Sacramento, CA (US); David Gregory CHAPIN, Sacramento, CA (US); Alexandria Marie Salazar CONNER, Sacramento, CA (US); Riley Anne DREXLER, Sacramento, CA (US); Megan LEE, Sacramento, CA (US); Nithya KRISHNAKUMAR, Sacramento, CA (US)

(73) Assignee: ONE BIO INC, Sacramento, CA (US)

(21) Appl. No.: 19/124,994

(22) PCT Filed: Nov. 9, 2023
(86) PCT No.: PCT/US2023/079274

§ 371 Date: Apr. 28, 2025

Related U.S. Application Data
(60) Provisional application No. 63/383,464, filed on Nov. 11, 2022.

(51) Int. Cl. A61K 9/00

(52) U.S. Cl. CPC A61K 9/0019; A61K 9/0053; A61K 2300/00

(57) ABSTRACT

Provided are methods for treating glucose-related metabolic disorders in a subject. In some aspects, the methods comprise enterally administering an effective amount of a beta-glucan oligosaccharide prior to and/or during the subject consuming a glucose source.

### CROSS-REFERENCE TO RELATED APPLICATIONS

This application claims the benefit of and priority to U.S. Provisional Patent Application No. 63/383,464 filed Nov. 11, 2022, which is hereby incorporated by reference in its entirety.

This application incorporates each of the following by reference for its entirety for all purposes, to the extent not inconsistent with the present disclosure: PCT Patent Application No. PCT/US22/29065, filed May 12, 2022 (WO 2022/241163 A1), PCT Patent Application No. PCT/US2018/038350, filed Jun. 19, 2018 (WO 2018/236917 A1), PCT Patent Application No. PCT/US2020/035748, filed Jun. 2, 2020 (WO 2020/247389 A1), and PCT Patent Application No. PCT/US2020/060297, filed Nov. 12, 2020 (WO 2021/097138 A1).

### FIELD

The disclosure herein generally relates to oligosaccharides, compositions comprising oligosaccharides, methods to obtain oligosaccharides and oligosaccharide compositions, methods for modulating microbiota and their metabolic products using oligosaccharides or oligosaccharide compositions, and methods for use of oligosaccharides or oligosaccharides as therapeutics for health applications, including for management of glucose levels in the blood.

### BACKGROUND

Chronic medical conditions or diseases are conditions that endure for extended periods and require ongoing medical attention or limit activities of daily living, or both. In some cases, the symptoms may go through phases of flare-ups and relapses while in other cases, the symptoms remain consistently. Chronic diseases are extremely prevalent and are a major contributor to impaired quality of life and health economic burdens. For example, chronic diseases such as heart disease, cancer, and diabetes are the leading causes of death and disability in the United States and are the leading drivers of the country's $3.8 trillion in annual health care costs.

Hyperglycemia, or high blood sugar, over time can lead to serious damage to many of the body's systems, especially the nerves and blood vessels. Long-term complications from hyperglycemia can include heart disease, strokes, diabetic retinopathy, kidney failure, and poor blood flow in the limbs. Lowering glycemic response is linked to a reduced risk of diabetes, which is characterized by chronic hyperglycemia. Postprandial blood sugar levels are related to insulin produced by the body and glucose absorbed in the small intestine and influenced by diverse environmental factors such as diet. Western diet, with high amount of high-sugar and high-starch foods, as well as changes in gut microbial community due to low fiber diets, can affect diverse mechanisms related to glucose absorption in our bodies. Most current therapeutic approaches aim at treating the consequences of hyperglycemia rather than causes of impaired metabolism. This approach is not efficient and therefore there remains a need for avenues that address potential causes or at least manage impaired metabolism over the longer term, and which are safe with little or no adverse side effects.

One strategy to control glucose level in blood is targeting mechanisms that affect glucose absorption at the interface between the gut wall and the lumen. Controlling the intestinal absorption of glucose in the brush-border membrane of the gut would help to reduce hyperglycemia risk. The glucose transporter sodium-glucose-linked transport protein (SGLT1) is responsible for the active transport of glucose across the brush border membrane of the small intestine, and inhibition of SGLT-1 can significantly reduce intestinal glucose absorption in patients at risk of diabetes. It has been reported that some polysaccharides remarkably reduced SGLT-1 expression not only in the intestinal mucosa of diabetic mice, but also in Caco-2 cells. Furthermore, a pure β-glucan, described as a linear β-(1→3)-glucan, from Baker's yeast is reported to significantly promote blood glucose regulation in normal mice, and reduced glucose transportation through Caco-2 monolayers (Cao et al. 2016).

A different strategy to reduce carbohydrate uptake from food into the blood is targeting alpha-glucosidases enzymes in the brush border membrane of the small intestine. Starch in the diet is cleaved in the stomach to small carbohydrate chains by alpha-amylases, and later in the small intestine into monosaccharides by enzyme complexes called alpha-glucosidases. By using compounds that are structurally like short starch derived carbohydrates, alpha-glucosidases can be competitively inhibited. Thus, monosaccharide formation decreases, and less glucose is absorbed by the host, leading to a reduction of food induced postprandial increases in blood glucose (Benalla, W et al. 2010). The antidiabetic drug Acarbose (a pseudo-tetrasaccharide, O-4,6-dideoxy-4-[[(1S,4R,5S,6S)-4,5,6-trihydroxy-3-(hydroxymethyl)-2-cyclohexen-1-yl]amino]-α-d-glucopyranosyl-(1→4)-O-α-d-glucopyranosyl(1→4)-d-glucose, is an alpha-glucosidase inhibitor. However, gastrointestinal side effects have been reported, in particular, flatulence and diarrhea, due to the fact that it also inhibits alpha-amylase activity.

Moreover, specific oligosaccharides undigested by the host can reach lower parts of the intestine where they are fermented by the gut microbiome. Fermentation of these oligosaccharides produce short chain fatty acids by specific bacterial microbes, which can activate GPR41 and GPR43 receptors. Activation of these receptors results in stimulating glucagon-like peptide-1 (GLP-1) secretion. This intestinal hormone delays emptying of the stomach, reducing glucagon secretion and regulating insulin secretion (Everard and Cani, 2014).

Optimal glycemic control is fundamental to the management of prediabetes and diabetes. Both fasting plasma glucose and postprandial plasma glucose levels correlate with the risk of complications and contribute to the measured glycated hemoglobin (A1C) value. A1C levels >7.0% are associated with a significantly increased risk of both microvascular and cardiovascular (CV) complications. Postprandial plasma glucose is an important component of overall hyperglycemia and may be the predominant component in patients who are closer to A1C limit and in older adults. For example, postprandial plasma glucose has been shown to be the main contributor to total glucose fluctuations in T2D patients with HbA1c <8%, i.e. well controlled diabetes or prediabetes patients. Prediabetes is rapidly increasing worldwide and is mainly associated with age and BMI. Therefore controlling postprandial plasma glucose in the overweight and obese population may play a role in preventing prediabetes from progressing to diabetes.

Current treatments for attenuating postprandial glucose response generally involve alpha-glucosidase inhibitor drugs such as Acarbose, or SGLT-1 inhibitor drugs such as such as Empagliflozin, Canagliflozin and Dapagliflozin. However, these drugs have side effects. Other approaches have involved use of nutritional formulas containing complex carbohydrates as meal replacements for foods containing simple carbohydrates. However, the need to consume a standard meal replacement usually leads to taste fatigue and loss of compliance over time.

Accordingly, there remains a need for safe, effective interventions which enable subjects to manage glucose response, particularly postprandial glucose response, over time.

### SUMMARY

Provided are methods for modifying microbial communities and the associated bioactive metabolites in vitro and/or in the gastrointestinal tract of a subject by applying one or more oligosaccharide compositions of varying structures. Depending on the oligosaccharide structures, microbial community structure (e.g., microbial abundance levels and composition) and metabolism respond in different ways. Also provided are methods of treating diseases, conditions, disorders, and/or indications relating to gastrointestinal health, and/or metabolic disorders. Also provided are novel oligosaccharide compositions and their structural features. Generally, the oligosaccharide compositions are, but need not be, derived from natural products.

Additionally provided are improved methods for generating polysaccharide cleavage products and/or oligosaccharide mixtures by reacting polysaccharides in a reaction mixture with a Fenton's reagent, having a peroxide agent and metal ions, and cleaving the treated polysaccharides with a base to generate polysaccharide cleavage products and/or oligosaccharide mixtures. In embodiments, improvements include use of copper metal ions in combination with hydrogen peroxide to achieve increased reaction efficiency and increased yield of cleavage products and oligosaccharide mixtures.

In one aspect, the disclosure provides a method for treating glucose-related metabolic disorders in a subject, the method comprising enterally administering to the subject an effective amount of a beta-glucan oligosaccharide prior to and/or during the subject consuming a glucose source. In a more specific aspect, the glucose-related metabolic disorder is pre-diabetes, diabetes, including type 1 diabetes, type 2 diabetes, gestational diabetes or metabolic syndrome.

In another aspect, the disclosure provides a method for attenuating post-prandial glucose response in a subject, the method comprising enterally administering to the subject an effective amount of a beta-glucan oligosaccharide prior to and/or during the subject consuming a glucose source. In a more specific aspect, the subject being treated is at risk of developing diabetes, is overweight and/or obese, is pregnant, and/or is prediabetic or diabetic.

In another aspect, the disclosure provides a method for reducing the risk of a prediabetic and/or obese subject from progressing to type 2 diabetes, the method comprising attenuating post-prandial glucose response in the subject by enterally administering to the subject an effective amount of a beta-glucan oligosaccharide prior to and/or during the subject consuming a glucose source.

In another aspect, the disclosure provides a method for lowering HbA1c levels in a subject, the method comprising attenuating the post-prandial glucose response in the subject over a period of at least 2 months by enterally administering to the subject an effective amount of a beta-glucan oligosaccharide prior to and/or during the subject consuming a glucose source.

In embodiments of the previous aspects of the disclosure, the subject is at risk of developing diabetes, is overweight and/or obese, is pregnant, and/or is prediabetic or diabetic. In further embodiments, the subject can be administered about 0.5 g to about 20 g of the beta-glucan oligosaccharide. For example, the subject can be administered about 0.75 g to about 15 g or about 0.75 g to about 7.5 g. of the beta-glucan oligosaccharide. In further embodiments, the subject can be administered the beta-glucan oligosaccharide less than about 2 hours, 1 hour or 30 minutes prior to the subject consuming the glucose source, for example less than about 15 minutes prior to the subject consuming the glucose source. In further embodiments, the beta-glucan oligosaccharide is co-administered with the glucose source. In some embodiments, the beta-glucan oligosaccharide and the glucose source are administered at or near the same time. In some embodiments, the beta-glucan oligosaccharide is administered prior to or at the same time as the glucose source. In some embodiments, the beta oligosaccharide and the glucose source are administered as part of the same treatment but are administered at different times, such as 8 hours apart, 12 hours apart, 1 day apart, 2 days apart, or 1 week apart. In further embodiments, the beta-glucan oligosaccharide inhibits salivary and/or pancreatic amylase. In further embodiment, the beta-glucan oligosaccharide inhibits the SGLT1 glucose transporter and/or inhibits alpha-glucosidase. In further embodiments, the beta-glucan oligosaccharide contains β-1,3 or β-1,4 linked glucose residues. In further embodiments, the beta-glucan oligosaccharide contains both β-1,3 and β-1,4 linked glucose residues. In further embodiments, the beta-glucan oligosaccharide comprises β-1,3: β-1,4 linked glucose residues in a ratio of 1:1 to 1:5; for example 1:1, or 1:2, or 1:3, or 1:4, or 1:5. In further embodiments, the beta-glucan oligosaccharide has a mean molecular weight of less than 10,000 da, or less than 8,000 da, or less than 7,500 da, or less than 5,000 da, optionally greater than 500 da, or greater than 1,000 da, or greater than 2,000 da. In further embodiments, the beta-glucan oligosaccharide contains 3 to 30 subunits, wherein each subunit is either a β-1,3 or α-1,4 glucose residue. In further embodiments, the beta-glucan oligosaccharide contains 3 to 30 subunits, wherein each subunit is either a β-1,3 or a β-1,4 glucose residue and each oligosaccharide contains both a β-1,3 and a β-1.4 glucose residue. In further embodiments, the beta-glucan oligosaccharide contains 3 to 30, or 3 to 25, or 5 to 30, or 5 to 25 subunits, or any subrange thereof, wherein each subunit is either a β1,3 or a β-1,4 glucose residue. In further embodiments, the beta-glucan oligosaccharide contains 3 to 30, or 3 to 25, or 5 to 30, or 5 to 25 subunits, or any subrange thereof, wherein each subunit is either a β-1,3 or a β-1,4 glucose residue and each oligosaccharide contains both a β-1,3 and a β-1,4 residue. In further embodiments, the beta-glucan oligosaccharide has a dynamic viscosity ranging from about 1 to about 10 mPa*s at 100 mg/ml at 25° C. In further embodiments, the beta-glucan oligosaccharide has a dynamic viscosity ranging from about 1 to about 5 mPa*s at 100 mg/ml at 25° C. or from about 1 to about 3 mPa*s at 100 mg/ml at 25° C. or from about 1 to about 1.5 mPa*s at 100 mg/ml at 25° C. or from about 1.3 to about 1.4 mPa*s at 100 mg/ml at 25° C., or any subrange thereof.

In yet another aspect, this invention provides a synthetic composition for treating a glucose-related metabolic disorder, attenuating post-prandial glucose response in a subject, reducing the risk of a prediabetic and/or obese subject from progressing to type 2 diabetes, and/or for lowering HbA1c levels in a subject, the synthetic composition comprising, consisting essentially of or consisting of an effective amount of a beta-glucan oligosaccharide. In further embodiments, the beta-glucan oligosaccharide contains-1,3 and β-1,4 linked glucose residues. In further embodiments, the beta-glucan oligosaccharide contains both β-1,3 and β-1,4 linked glucose residues. In further embodiments, the beta-glucan oligosaccharide comprises β-1,3: β-1,4 linked glucose residues in a ratio of 1:1 to 1:5; for example 1:1, or 1:2, or 1:3, or 1:4, or 1:5, or any subrange thereof. In further embodiments, the beta-glucan oligosaccharide mixture has a mean molecular weight of less than 10,000 da or less than 8,000 da, or less than 7,500 da, or less than 5,000 da, or any subrange thereof. In further embodiments, the beta-glucan oligosaccharide contains about 3 to about 50 subunits, wherein each subunit is either a β-1,3 or a β-1,4 glucose residue. In further embodiments, the beta-glucan oligosaccharide contains about 3 to about 50 subunits, wherein each subunit is either a β-1,3 or a β-1,4 glucose residue and each oligosaccharide contains both a β-1,3 and a β-1,4 glucose residue. In further embodiments, the beta-glucan oligosaccharide contains 3 to 30, or 3 to 25, or 5 to 30, or 5 to 25 subunits, wherein each subunit is cither α-1,3 or a β-1,4 glucose residue. In further embodiments, the beta-glucan oligosaccharide contains 3 to 30, or 3 to 25, or 5 to 30, or 5 to 25 subunits, wherein each subunit is either a β-1,3 or a β-1,4 glucose residue and each oligosaccharide contains both a β-1,3 and a β-1,4 residue. In further embodiments, the beta-glucan oligosaccharide has a dynamic viscosity ranging from about 1 to about 10 mPa*s at 100 mg/ml at 25° C. In further embodiments, the beta-glucan oligosaccharide has a dynamic viscosity ranging from about 1 to about 5 mPa*s at 100 mg/ml at 25° C. or from about 1 to about 3 mPa*s at 100 mg/ml at 25° C. or from about 1 to about 1.5 mPa*s at 100 mg/ml at 25° C. or from about 1.3 to about 1.4 mPa*s at 100 mg/ml at 25° C.

In a yet further aspect, this invention provides a pack for use for treating a glucose-related metabolic disorder, or for attenuating post-prandial glucose response in a subject, or reducing the risk of a prediabetic and/or obese subject from progressing to type 2 diabetes, and/or for lowering HbA1c levels in a subject, the pack comprising, consisting essentially of or consisting of, at least 14 individual doses of an effective amount of a beta-glucan oligosaccharide, wherein the beta-glucan oligosaccharide is as described herein above.

In an additional aspect, the disclosure provides use of a beta-glucan oligosaccharide as described herein above for treatment of a glucose-related metabolic disorder, or for attenuating post-prandial glucose response in a subject, or reducing the risk of a prediabetic and/or obese subject from progressing to type 2 diabetes, and/or for lowering HbA1c levels in a subject.

In an additional aspect, the disclosure provides use of a beta-glucan oligosaccharide as described herein above for the preparation of a medicament for treatment of a glucose-related metabolic disorder, or for attenuating post-prandial glucose response in a subject, or reducing the risk of a prediabetic and/or obese subject from progressing to type 2 diabetes, and/or for lowering HbA1c levels in a subject.

In embodiments, the glucose-related metabolic disorders herein include among others, diabetes (type 1, type 2 and gestational) as well as prediabetes and metabolic syndrome.

Additional aspects and embodiments of the disclosure will be apparent to one of ordinary skill in the art on review of the following detailed description, non-limiting examples and drawings.

Without wishing to be bound by any particular theory, there may be discussion herein of beliefs or understandings of underlying principles relating to the devices and methods disclosed herein. It is recognized that regardless of the ultimate correctness of any mechanistic explanation or hypothesis, an embodiment or aspect of the invention can nonetheless be operative and useful.

### BRIEF DESCRIPTION OF THE DRAWINGS

FIG. 1: Relative transporter specific accumulation (%) of AMG.

FIG. 2: Alpha-glucosidase inhibition by CLX115.

FIG. 3: Alpha-glucosidase inhibition by different beta-glucan sources.

FIGS. 4A-4B: In vitro butyrate level production by CLX115 (SHIME model). FIG. 4A depicts the proximal colon results. FIG. 4B depicts the distal colon results.

FIGS. 5A-5B: In vitro propionate level production by CLX115 (SHIME model). FIG. 5A depicts the proximal colon results. FIG. 5B depicts the distal colon results.

FIG. 6: Gas production levels (kPa).

FIGS. 7A-7D: RID chromatograms. FIG. 7A depicts results for CLX115-PS. FIG. 7B depicts results for CLX115Cu. FIG. 7C depicts results for CLX115. FIG. 7D depicts results for CLX112.

FIGS. 8A-8B: Evaluation of alpha-glucosidases inhibition by CLX115. FIG. 8A depicts the concentration levels of maltose over time. FIG. 8B depicts the concentration of glucose over time.

FIGS. 9A-9B. In vivo evaluation of blood glucose levels. In both FIG. 9A and FIG. 9B, “ctrl” refers control, where maltose was administered alone. FIG. 9A depicts the blood glucose levels measured at different time points after carbohydrate gavage of two groups of mice (n=15/group).

FIG. 9B depicts the peak blood glucose levels at 30 minutes after carbohydrate gavage.

### Statements Regarding Chemical Compounds and Nomenclature

In general, the terms and phrases used herein have their art-recognized meaning, which can be found by reference to standard texts, journal references and contexts known to those skilled in the art. The following definitions are provided to clarify their specific use in the context of the invention.

The following abbreviations are used herein: Deoxyhex refers to deoxyhexose sugar; DP refers to degrees of polymerization; Glc refers to glucose; Hex refers to hexose sugar; HexA refers to hexuronic acid sugar; Man refers to mannose; Pent refers to pentose; and PS refers to polysaccharide.

As used herein, the terms “about” and “approximately.” when used to modify an amount specified in a numeric value or range, mean that slight variations from a stated value may be used to achieve substantially the same results as the stated value. In circumstances where this definition cannot be applied or is exceedingly difficult to apply, then the terms “about” and “approximately” mean a reasonable deviation from the value known to a skilled person in the art, such as, if “X” were the value, for example, “about X” or “approximately X” would indicate a value from 0.9X to 1.1X, e.g., a value from 0.95X to 1.05X, or a value from 0.98X to 1.02X, or a value from 0.99X to 1.01X. Any reference to “about X” or “approximately X,” where X is a value disclosed herein, specifically indicates at least the values X, 0.9X, 0.91X, 0.92X, 0.93X, 0.94X, 0.95X, 0.96X, 0.97X, 0.98X, 0.99X, 1.01X, 1.02X, 1.03X, 1.04X, 1.05X, 1.06X, 1.07X, 1.08X, 1.09X, and 1.1X, and values within this range.

As used herein, the terms “collectively comprise,” “collectively comprises,” or similar terms, when in reference to one or more oligosaccharides (or any similar context), means that the one or more oligosaccharides as a whole have the indicated composition or property. For example, a given composition can contain two different oligosaccharides (e.g., a first oligosaccharide comprising glucose subunits but not arabinose subunits, and a second oligosaccharide comprising arabinose subunits but not glucose subunits). In this situation, the two different oligosaccharides (i.e., the “one or more oligosaccharides”) collectively comprise glucose and arabinose subunits. Similarly, the term “collectively” has the same meaning in any similar context (e.g., in reference to a composition, etc.) to indicate that any components that are present collectively have an indicated feature or property (e.g., NMR analysis or linkage types or amounts, etc.).

Any viscosity measurement or property reported herein employs water as the solvent, unless specified otherwise.

In some aspects, a composition or compound disclosed herein, such as an oligosaccharide or oligosaccharide composition, is isolated or substantially purified. In an embodiment, an isolated or purified oligosaccharide or oligosaccharide composition is at least partially isolated or substantially purified as would be understood in the art. In some aspects, a substantially purified composition, oligosaccharide or formulation disclosed herein has a chemical purity of 95%, optionally for some applications 99%, optionally for some applications 99.9%, optionally for some applications 99.99%, and optionally for some applications 99.999% pure.

As used herein, the term “polysaccharide” refers to a polysaccharide or a material comprising a polysaccharide, in either case wherein at least the polysaccharide component is cleavable by the COG methods disclosed herein. Additionally, as used herein, the term “polysaccharide” refers to any carbohydrate polymer and can also be linked to other non-carbohydrate moieties (e.g., glycoproteins, proteoglycans, glycopeptides, glycolipids, glycoconjugates, glycosides, or any combination thereof). Moreover, as used herein “polysaccharide” refers to a polymer of monosaccharide units of greater than 30 monosaccharide units, and can reach hundreds of thousands of monosaccharides in length. A polysaccharide can be a linear polymer, branched polymer, primarily linear polymer with pendant saccharide monomers, or any combination thereof.

As used herein, the term “peroxide agent” refers to compounds that contain oxygen-oxygen bonds that can produce, natively, with light, temperature, or catalyst (e.g., metals and enzymes), R—O⁻ and/or R—O—O⁻ species, where “R” refers to a hydrogen or carbon group that is attached to the rest of the molecule. In one aspect, a peroxide agent is hydrogen peroxide.

The “degree of polymerization” or “DP” of an oligosaccharide refers to the total number of sugar monomer units (also referred to herein as subunits) that are part of a particular carbohydrate. For example, a tetra galacto-oligosaccharide has a DP of 4, having 3 galactose moieties and one glucose moiety. When used to describe a group of oligosaccharides (e.g., an oligosaccharide composition), DP generally refers to the mean DP of the oligosaccharides in the composition. In some aspects, the DP of an oligosaccharide is referred to as “DP #”, where “#” corresponds to an integer representing the total number (or average number if used to describe a group of oligosaccharides) of sugar monomer units (e.g., “DP3” means a degree of polymerization of 3). For oligosaccharides or oligosaccharide compositions discussed herein having a DP between the range of 3-9, the recited DP includes a variance of up to ±2 monomer units of the recited value. For oligosaccharides or oligosaccharide compositions having a DP of 10 or greater, the recited DP includes a variance of up to +20% of the recited value, for example, an oligosaccharide having a DP of 20 may have a DP of 16, 17, 18, 19, 20, 21, 22, 23, or 24.

As used herein, when the amount of a component is expressed in terms of weight or mole percent, it is intended that the amount is on a dry basis unless otherwise specified. As used herein, “dry basis” means in the absence of water or other solvent. For example, when a composition comprises 10 g of glucose, 40 g of xylose, and 50 g of water, it means the composition comprises 25% (mass % or wt. %) glucose on a dry basis, but the glucose is present in the composition at a concentration of 10% (mass % or wt. %).

As used herein, “live biotherapeutic” refers to a therapeutic or medicinal product that comprises a living microorganism, such as an archaeon, a bacterium, an algae, a fungus (e.g., a yeast), or any combination thereof, as an active ingredient. In some aspects, the live biotherapeutic may comprise an engineered (e.g., genetically modified) or un-engineered living organism, or a mixture of both engineered and un-engineered living organisms.

A “prebiotic” or “prebiotic nutrient” is generally a non-digestible or partially-digestible (i.e., digestible by the subject/human/animal, and does not include digestion by microbes) food ingredient that beneficially affects a host when ingested by selectively stimulating the growth and/or the activity of one or a limited number of microbes in the gastrointestinal tract, urogenital system, or other portion of the host. As used herein, the term “prebiotic” refers to the above described non-digestible or partially-digestible food ingredients in their non-naturally occurring states, e.g., after purification, chemical or enzymatic synthesis as opposed to, for instance, in whole human milk.

A “probiotic” refers to live microorganisms that when administered in adequate amounts confer a health benefit on the host.

As used herein, a “peeling reaction” or “peeling” as applied to the disclosed methods refers to the sequential alkaline degradation of carbohydrates through a mechanism that releases monomeric units from the reducing end of the polymer.

As used herein, a “cleavage agent” or “cleavage reagent” as applied to the disclosed methods preferably refers to a single or collection of non-Arrhenius and/or weak-Arrhenius bases used to cleave polysaccharides after hydroperoxyl oxidation thereof. In certain aspects, a cleavage agent or cleavage reagent breaks glycosidic bonds in the polysaccharide, which bonds may be present between any two saccharides of the polysaccharide. In embodiments, the cleavage reagent (cleavage initiator) may also be, and preferably is a peroxide-quenching reagent, and in either case may be used in combination with an additional compatible peroxide-quenching agent that may or may not also be a cleavage agent. In some aspects, a cleavage reagent may be an enzyme. In some aspects, the cleavage reagent enzyme may be a glycosyl hydrolase, a lytic polysaccharide monooxygenase, a glycosyl transferase, transglycosidase, polysaccharide lyase, carbohydrate binding module, glucosyl transferase, carbohydrate esterase, a cocktail containing two or more of the aforementioned enzymes, or any enzyme that is carbohydrate active. In some aspects, a cleavage reagent may be a solid-phase acid catalyst or a solid-phase base catalyst.

As used herein, a “base” refers to a compound or collection of compounds that can accept hydrogen ions from the peroxyl oxidized carbohydrate, water, or non-aqueous solvent. The term “base” can include Lewis bases, non-Arrhenius bases, weak-Arrhenius bases, other molecules that produce through their decomposition hydroxide ions, Lewis bases, non-Arrhenius bases, or weak-Arrhenius bases, or other compounds that can accept hydrogen ions from the hydroperoxyl oxidized carbohydrate. As used herein, unless otherwise specified, a “base” explicitly does not refer to a strong-Arrhenius base (e.g., Na⁺OH⁻, K⁺OH⁻, or Ca⁺²(OH⁻)₂).

As used herein, “ammonium bicarbonate” as applied to the disclosed methods refers to solid ammonium bicarbonate, and/or an aqueous solution containing: ammonium and bicarbonate: ammonium, OH⁻, and CO₂; ammonia, H₂O, and CO₂; or any of the preceding and their equilibrium products.

As used herein, “ammonium hydroxide” as applied to the disclosed methods refers to: aqueous ammonium hydroxide, and/or a solution containing: ammonia and H₂O; ammonium and OH⁻; ammonia and OH⁻; or any of the preceding and their equilibrium products.

As used herein, a “strong-Arrhenius base” as applied to the disclosed methods refers to a compound that completely dissociates in water to release one or more hydroxide ions into solution. As used herein, a “strong-Arrhenius base” as applied to the disclosed methods refers explicitly to KOH, NaOH, Ba(OH)₂, CsOH, Sr(OH)₂, Ca(OH)₂, LiOH, and RbOH.

As used herein, a “weak-Arrhenius base” as applied to the disclosed methods refers to a compound that incompletely dissociates in water to release one or more hydroxide ions into solution, e.g. ammonium hydroxide, H₂O, etc. As “weak-Arrhenius base” is used herein, there are no compounds which meet both the definition of strong-Arrhenius base and weak-Arrhenius base.

As used herein, a “non-Arrhenius base” as applied to the disclosed methods refers to a compound or atom that can donate electrons (e.g., Lewis Bases), accept protons (e.g., Bronsted-Lowry Bases), or releases hydroxide ions through its decomposition (NH₄HCO₃), but explicitly does not qualify as an Arrhenius base.

As used herein, a “Lewis base” as applied to the disclosed methods refers to a compound or atom that can donate electron pairs (e.g., F″, benzene, H″, pyridine, acetonitrile, acetone, urea, etc.).

As used herein a “Bronsted-Lowry base” as applied to the disclosed methods refers to a compound or atom that can accept or bond to a hydrogen ion (e.g., methanol, formaldehyde, ammonia, etc.).

As used herein a “Peroxide quenching reagent” as applied to the disclosed methods refers to a compound or atom, which is not a strong-Arrhenius base, that can convert hydrogen peroxide, peroxyl radicals, and hydroperoxyl radicals to a less reactive or non-reactive state (e.g., ammonium hydroxide, ammonium bicarbonate, ammonia, etc.). In certain aspects, a peroxide quenching reagent as defined herein converts hydrogen peroxide as well as radicals produced from hydrogen peroxide to less reactive species (e.g. water). In certain aspects, a peroxide quenching reagent may reduce the hydrogen peroxide concentration to zero, below 5 mg/L, below 10 mg/L, below 25 mg/L, or below 50 mg/L. In certain aspects, a peroxide quenching reagent may form water, hydroxide ions, or oxygen gas. In certain aspects, enzymes may be used to quench peroxide species. In certain aspects, those enzymes may include catalases. In certain aspects, those enzymes can be from animal origin. In certain aspects, those enzymes can be from bovine liver. In certain aspects, the enzymes may be from microbial origin. In certain aspects, the enzyme may be recombinant. In certain aspects, different enzymes may be mixed to quench the peroxide species.

As used herein “nitrogen-based” as applied to the disclosed methods refers to a compound that contains at least one nitrogen atom with four substituent groups that can contain any combination of lone pairs of electrons, hydrogens, or carbon atoms (e.g., ammonia, sodium amide, trimethylamine, diethylamine, N,N-Diisopropylethylamine, urea, pyridine, ammonium hydroxide, ammonium bicarbonate, etc.). Exemplary nitrogen-based, peroxide-quenching, PS-cleavage agents are listed in Table 1 below. A nitrogen-based reagent may have an unsubstituted or substituted ammonium group and can be present in neutral and/or ionic forms.

TABLE 1
Exemplary polysaccharide (PS)-cleavage,
and/or peroxide-quenching agents.

| | | |
|---|---|---|
| Compound | Peroxide-quenching | Cleavage |

Exemplary nitrogen-based, peroxide-quenching, PS-cleavage agents

| | | |
|---|---|---|
| Ammonium Hydroxide | Yes | Yes |
| Ammonium Bicarbonate | Yes | Yes |
| Ammonia | Yes | Yes |
| Urea | Yes | Yes |
| Sodium Amide | Yes | Yes |
| Trimethyl amine | (Yes) | Yes |
| Diethylamine | (Yes) | Yes |
| Pyridine | (Yes) | Yes |
| N,N-Diisopropylethylamine | (Yes) | Yes |

Exemplary Lewis base, peroxide-quenching, PS-cleavage agents

| | | |
|---|---|---|
| F— | Yes | Yes |
| H— | Yes | Yes |
| Methoxide (CH₃O⁻) | Yes | Yes |
| Ethoxide (C₂H₅O⁻) | Yes | Yes |
| Tertbutoxide (C₄H₉O⁻) | Yes | Yes |

Exemplary strong Arrhenius base, non-peroxide-quenching, PS-cleavage
agents

| | | |
|---|---|---|
| Sodium Hydroxide | No | Yes |
| Potassium Hydroxide | No | Yes |
| Calcium Hydroxide | No | Yes |
| Cesium Hydroxide | No | Yes |
| Strontium Hydroxide | No | Yes |
| Lithium Hydroxide | No | Yes |
| Rubidium Hydroxide | No | Yes |

Exemplary peroxide-quenching, non-PS-cleavage agents

| | | |
|---|---|---|
| Methanol | Yes | No |
| Acetonitrile | Yes | No |
| Formaldehyde | Yes | No |
| Chlorine Gas | Yes | No |
| Salts of Sulfite | Yes | No |
| Salts of Thiosulfite | Yes | No |
| Catalase Enzyme | Yes | No |

As used herein “reaction mixture” refers to a mixture comprising reagents which may react chemically to form products which are distinct from the reagents.

As used herein “treated polysaccharide” refers to a polysaccharide which has been contacted with at least one reagent capable of reacting with the polysaccharides (e.g. an enzyme or a Fenton's reagent).

As used herein “polysaccharide cleavage product” is a product formed from the chemical and/or enzymatic cleavage of a polysaccharide. In some aspects, the polysaccharide cleavage product comprises one or more oligosaccharides. In some aspects, the polysaccharide cleavage product comprises one or more polysaccharides. In some aspects, the polysaccharide cleavage product comprises a mixture of one or more oligosaccharides and one or more polysaccharides.

As used herein “oligosaccharide” refers to an oligomer of saccharides, in which the DP of the oligomer is between 2 and 30 monosaccharide units, such as between 3-30, 3-20, 3-25, 3-15, 3-10, 3-8, 3-6, or 5-15, or any subrange thereof, monosaccharide units. An oligosaccharide can be linear, branched, primarily linear with pendant saccharide monomers, or any combination thereof. An “oligosaccharide” refers to an individual oligomer chain.

As used herein, “oligosaccharide composition” (also termed “oligosaccharide pool” or oligosaccharide mixture” herein) refers to a mixture of two or more oligosaccharides, each of which can be the same or different from one another. Although efforts have been made herein to consistently use the terms “oligosaccharide” and “oligosaccharide composition” according to their preceding definitions, the intended meaning will be clear from context when such terms are used herein. In embodiments, “one or more oligosaccharides” refers to an oligosaccharide mixture when more than one oligosaccharide is present. In embodiments, “one or more oligosaccharides” refers to one oligosaccharide. In some embodiments, an oligosaccharide composition comprises one or more polysaccharides. In keeping with this aspect, an oligosaccharide composition may comprise up to 60-80% polysaccharides by mass, preferably less than 70% or less than 60% polysaccharides by mass (e.g., between 0.5% and 70% polysaccharides, between 0.5% and 60% polysaccharides, or between 0.5% and 50% polysaccharides). In preferred embodiments, an oligosaccharide composition comprising up to 60-80% polysaccharides has a higher solubility, increased bioactivity, or a combination thereof, as compared to a composition comprising between 80-100% polysaccharides.

As used herein, “subunit” (sometimes referred to as “unit” or “residue” herein) means a species that is covalently bonded to or within an oligomer (e.g., oligosaccharide) or polymer (e.g., polysaccharide). In some aspects, such species generally can include saccharides (e.g., glucose, galactose, mannose, etc.). For example, when an oligosaccharide composition comprises a glucose subunit, it means that the composition comprises a glucose molecule that is bound to or within an oligomer or polymer; as such, a composition that contains only free monomeric glucose would not contain a glucose subunit. Similarly, when an oligosaccharide composition comprises a sum of glucose, galactose, and mannose subunits in an amount of at least 60 wt. % based on total weight of saccharide subunits, this means that the mass of all of the glucose subunits, galactose subunits, and mannose subunits are summed, and the subunits of all saccharides are summed, and then the first sum is divided by the second sum. Additionally, when an oligosaccharide composition comprises non-terminal galactose subunits, and at least 70 wt. % of the non-terminal galactose subunits are specified to have at least one 4-linkage, this feature is calculated by summing the mass of all non-terminal galactose subunits having at least one 4-linkage (and this can include, for example, galactose subunits with 4,6-linkages and 4,3-linkages), and then dividing by the total mass of non-terminal galactose subunits regardless of linkage type. The same concept is applicable to any feature herein where reference to “at least one X-linkage,” in which X is an integer (e.g., such as “a weight ratio of glucose subunits having at least one 4-linkage to glucose subunits having at least one 3-linkage is between 2:1 to 4:1” and other such features). Moreover, in such calculations the actual mass of the subunit is used (i.e., in bound form) rather than the mass of the unit as if it was hydrolyzed (which would add the mass of water). Other features described elsewhere herein can be calculated similarly. These features can be determined with the aid of the various analytical techniques described herein, such as hydrolytic monosaccharide compositional analysis, oligosaccharide analysis, glycosidic linkage analysis, NMR HSQC Analysis, and so forth, as well as other techniques known in the art.

As used herein “Fenton's reagent” refers to a reagent comprising a peroxide agent and a metal. In certain aspects, the peroxide agent is hydrogen peroxide. In certain aspects, the metal is Fe(II), Fe(III). Cu(I), Cu(II), Mn(II), Zn(II), Ni(II), and Co(II), alkaline earth metal ions Ca(II) and Mg(II), the lanthanide Ce(IV) or any combination thereof. In specific embodiments, the metal ion is a copper ion, and particularly is Cu(II).

As used herein the phrase “substantially commensurate with initiation of peroxide-quenching” refers to the relationship between the timing of a cleavage reaction and the timing of a peroxide quenching reaction indicating that the initiation of the cleavage reaction and the initiation of the peroxide quenching reaction occur within a short time duration of each other (e.g. on the order of seconds, or on the order of minutes but not more than one day).

As used herein “specified reaction time” or “reaction time” refers to providing time to allow a reaction to proceed toward an equilibrium state between reagents added and products produced by the reaction of the reagents. In certain aspects, specified reaction time allows sufficient time to reach an equilibrium. In certain other aspects, specified reaction time, while allowing time for the reaction to proceed toward equilibrium, does not provide the time needed to reach equilibrium.

As used herein, the term “synthetic oligosaccharide” refers to an oligosaccharide produced by the depolymerization of one or more polysaccharides. In certain aspects, the term synthetic oligosaccharide refers to compositions of oligosaccharides produced by the methods disclosed herein. The depolymerization to produce synthetic oligosaccharides can alternatively or additionally take place using enzymes, chemical reactions such as Fenton's chemistry, physical processes such as elevated time and temperature, and so forth, or any combination thereof. In some aspects, the term synthetic oligosaccharide refers to oligosaccharides prepared by synthesizing the oligosaccharide from monosaccharides or lower DP oligosaccharides. The term “synthetic oligosaccharide” is used interchangeably herein with “oligosaccharide,” and “composition comprising at least one synthetic oligosaccharide” is used interchangeably herein with “oligosaccharide composition.” No difference in meaning is intended.

As used herein, the term “synthetic composition” means a composition which is artificially prepared and preferably means a composition containing at least one compound that is produced ex vivo chemically and/or biologically, e.g., by means of chemical reaction, enzymatic reaction, recombinantly, or any combination thereof. The synthetic composition typically comprises one or more compounds, including one or more of the oligosaccharides described herein. In some aspects, the oligosaccharides and oligosaccharide compositions can be formulated into a synthetic composition or administered as the oligosaccharide alone. In some embodiments, the synthetic composition can be in the form of a nutritional composition or a pharmaceutical composition.

As used herein, the term “heteropolymer polysaccharide” refers to a polysaccharide containing two or more kinds of monosaccharide subunits linked together by the same type of glycosidic bond or different types of glycosidic bonds; heteropolymer polysaccharides also include polysaccharides containing repeating monosaccharide subunits of the same kind linked together by different types of glycosidic bonds. The glycosidic bonds in a heteropolymer polysaccharide may be β1-2 bonds, β1-3 bonds, β1-4 bonds, β1-5, β1-6 bonds, α1-3 bonds, α1-4 bonds. β1-5, α1-6 bonds, or a combination thereof. Examples of heteropolymer polysaccharides include, but are not limited to, xyloglucan, lichenan, β-glucan, glucomannan, galactomannan, arabinan, xylan, and arabinoxylan.

As used herein, “short chain fatty acid” includes butyrate, propionate, betahydroxybutyrate, lactate, acetate, or any combination thereof.

As used herein, the term “hydrolytic monosaccharide compositional analysis” refers to the method described in Amicucci, Galermo et al. 2019, hereby incorporated by reference in its entirety for all purposes to the extent not inconsistent with the description herein, with some modifications. In some aspects, the hydrolysis reaction to produce monosaccharides was performed at the optimized condition of 100° C. for 2 hours. Samples were ran on an Agilent 1290 Infinity II ultra-high performance liquid chromatography (UHPLC) system couple to an Agilent 6490A triple quadrupole (QqQ) mass spectrometer. Separation was carried out on an Agilent Infinity Lab Poroshell HPH-C18 column (2.1 mm×50 mm, 1.9 μm particle size) plus a guard column (5 mm) with the same solvent system described in Amicucci, Galermo et al. 2019. With a constant flow rate of 1.2 mL/min, an isocratic gradient of 8.5% B was used for the first 4-min elution period, followed by 15% B for 0.4 min. For the flush period, 97% B was held for 1 min. The column thermostat was set at 35° C. For the mass spectrometry parameters, the only change from the method described in Amicucci, Galermo et al. 2019 is that the fragmentor voltage was set at 380V. For data analysis, the hydrolysis correction factor was not applied nor needed since the samples contained oligosaccharides instead of polysaccharides. In this analysis method, monosaccharide composition is calculated by quantifying the concentrations of 14 monosaccharides (glucose, galactose, fructose, xylose, arabinose, fucose, rhamnose, glucuronic acid, galacturonic acid, N-acetylglucosamine, N-acetylgalactosamine, mannose, allose, ribose) against their individual standard curves. For example, 30% glucose, as measured by the herein hydrolytic monosaccharide compositional analysis, refers to containing 30 g of glucose per 100 g of the sum of all 14 monosaccharides described above.

As used herein, the term “free monosaccharide compositional analysis” refers to the method described in MJ Amicucci et al. 2019 (Amicucci, Galermo et al. 2019) with some modifications. The derivatization reaction to produce monosaccharides was performed at the optimized condition of 70° C. for 30 minutes. Samples were ran on an Agilent 1290 Infinity II ultra-high performance liquid chromatography (UHPLC) system couple to an Agilent 6490A triple quadrupole (QqQ) mass spectrometer. Separation was carried out on an Agilent Infinity Lab Poroshell HPH-C18 column (2.1 mm×50 mm, 1.9 μm particle size) plus a guard column (5 mm) with the same solvent system described in the paper. With a constant flow rate of 1.2 mL/min, an isocratic gradient of 8.5% B was used for the first 4-min elution period, followed by 15% B for 0.4 min. For the flush period, 97% B was held for 1 min. The column thermostat was set at 35° C. For the mass spectrometry parameters, the only change from the method described in the paper is that the fragmentor voltage was set at 380V. For data analysis, the hydrolysis correction factor was not applied since the samples herein contains oligosaccharides instead of polysaccharides. In this analysis method, inherently free unpolymerized monosaccharides are calculated by quantifying the concentrations of 14 monosaccharides (glucose, galactose, fructose, xylose, arabinose, fucose, rhamnose, glucuronic acid, galacturonic acid, N-acetylglucosamine, N-acetylgalactosamine, mannose, allose, ribose) against their individual standard curves. For example, 30% free glucose, as measured by the herein free monosaccharide compositional analysis, refers to containing 30 g of glucose per 100 g of the sum of all 14 monosaccharides described above.

As used herein, the terms “monosaccharide ratio,” “monosaccharide peak area ratio.” “ratio of monosaccharide,” or similar terms can refer to any number of the comparisons dependent upon the relationships observed in the hydrolytic monosaccharide compositional analysis. Absolute concentrations of each monosaccharide were calculated on a relative percent basis in relation to the summation of all other monosaccharides observed. Monosaccharide ratios were calculated by dividing one contributing monosaccharide by any other monosaccharide within the composition.

Monosaccharide ratios are not intended to limit the composition to the listed monosaccharides. For example, a glucose: galactose ratio of 1:1 means that there are roughly equal amounts of glucose subunits and galactose subunits in the composition, but the composition may also comprise mannose subunits, rhamnose subunits, or any other subunit.

As used herein, the terms “glycosidic linkage composition,” “glycosidic linkage analysis,” “permethylated linkage composition analysis,” or similar terms, refer to a method described in Galermo, Nandita et al. 2018, hereby incorporated by reference in its entirety for all purposes to the extent not inconsistent with the description herein, with some modifications. The permethylation reaction time was 30 min instead. Samples were ran on an Agilent 1290 Infinity II UHPLC system couple to an Agilent 6490A QqQ mass spectrometer. Separation was carried out on an Agilent Infinity Lab Poroshell HPH-C18 column (2.1 mm×100 mm, 1.9 μm particle size) plus a guard column (5 mm) with the same solvent system described in Galermo, Nandita et al. 2018. With a constant flow rate of 0.8 mL/min, an isocratic gradient of 14% B was used for the 16-min elution period, followed by a 2-min 99% B flush period. The column thermostat was set at 35° C. The glycosidic linkage composition is calculated by integrating the chromatographic peak area of all peaks with the following m/z values: 481.2, 495.2, 509.2, 523.3, 525.2, 537.3, 539.3, 553.3, 567.3, and 581.3. For example, 20% 4-galactose, as measured by the permethylated linkage composition analysis, refers to the peak area of 4-galactose being 20% of the sum of the peak area of all linkage peaks with the m/z values listed above.

As used herein, the term “other minor linkages” refers to the sum of linkages which are either not entirely annotated or constitute less than 2% of any samples. Therefore, the contributions of these linkages to the sample glycosidic linkage composition are summed into this “other minor linkages” category.

As used herein, the terms “linkage ratio,” “linkage peak area ratio,” “ratio of linkage,” or other similar terms can refer to any number of comparisons dependent upon the relationships observed in the glycosidic linkage composition analysis. For example, an oligosaccharide having a ratio of beta-1,3 linked to beta-1,4 linked residues ranging from 1:1 to 1:5 refers to a linkage ratio as measured by glycosidic linkage composition analysis. Peak area for each linkage was calculated on a relative percent basis of the peak area in relationship to the summation of all other linkage peaks areas observed. Peak area ratios are calculated by dividing one contributing linkage by any other linkage of the same monosaccharide within the composition.

As used herein, the terms “oligosaccharide analysis” or “oligosaccharide composition analysis” (or similar terms) refer to a HPLC-quadrupole time-of-flight (Q-TOF) method described in Amicucci, Nandita et al. 2020, hereby incorporated by reference in its entirety for all purposes to the extent not inconsistent with the description herein, with some modifications. For sample prep, oligosaccharides were reduced by incubation with 2.0 M NaBH4 for 1 h at 65° C. Oligosaccharides were purified using C-18 cartridge 96-well plates: plates were washed with 100% ACN, and the oligosaccharides were loaded and eluted with water. Oligosaccharides were subsequently purified using porous graphitized carbon (PGC) 96-well plates: PCG plates were washed with 80% acetonitrile and 0.1% (v/v) TFA in water, and the oligosaccharides from C-18 purification were loaded and washed with water. The oligosaccharides were eluted with 40% acetonitrile with 0.05% (v/v) TFA. Samples were completely dried by evaporative centrifugation and reconstituted for mass spectrometry analysis. Instrumentation was performed on an Agilent 1260 Infinity II HPLC coupled to an Agilent 6530 Q-TOF mass spectrometer. Using the same stationary (plus a 5 mm guard column) and mobile phase as described in the paper, separation was carried out using the following gradient: 2-15% B, 0-20 min: 15-60% B; 20-45 min. The column thermostat was set at 35° C. The fragmentor voltage was set at 75V. In this method, “oligosaccharide weight %” or “oligo wt. %” or such terms when used in the context of the “oligosaccharide analysis” was calculated by dividing the chromatographic peak area of a particular oligosaccharide by the total peak area of all oligosaccharides identified in that sample during the defined chromatographic period. Generally, when an oligosaccharide composition is described herein to contain a specified weight percent of oligosaccharides on a dry basis having a degree of polymerization of a specified number (e.g., at least 50 wt. % oligosaccharides on a dry basis having a degree of polymerization of between 3 and 30 monosaccharide subunits), such values can be calculated with the aid of the oligosaccharide analysis described herein; however, other methods can also aid this determination, such as size exclusion chromatography using a universal detector, or other methods known in the art.

As used herein, the term “retention factor” refers to the ratio obtained by dividing the retention time of a given peak observed in an oligosaccharide analysis (e.g., HPLC spectrum) by the first oligosaccharide peak (i.e., the lowest retention time) observed in the oligosaccharide analysis.

As used herein the terms, “NMR HSQC Analysis,” “1H-13C HSQC NMR,” “HSQC spectra” or other similar terms correspond to the data generated from two-dimensional spectral analysis of a sample via a Heteronuclear Single Quantum Coherence (HSQC) spin coupling of protons and bonded carbons present in said sample. HSQC experimentation depends on the solvation of samples in a deuterated solvent such as D6-DMSO or D20. An HSQC spectrum contains a unique peak for each proton attached to the heteronuclear carbon atom being considered, allowing for identification of molecular structure of analyzed sample. Each experiment was conducted with a Bruker AVANCE 600 MHz NMR using heteronuclear single quantum coherence (HSQC) to illustrate the correlation between the 1H and 13C chemical shifts through 1JCH coupling. The resulting FIDs were processed using Bruker TopSpin 4.1.3 and the experimental chemical shifts were utilized to determine oligosaccharide structures and the anomeric characteristics of the glycosidic bonds with the aid of the CASPER program. Relative ratios between alpha and beta bonds were calculated through examination of the 2D 1H-13C HSQC via examination of signal strength in Hz. These values were then compared to determine percent abundance of each linkage type among the same carbohydrate. NMR samples were dried via lyophilization, and the resulting material were then dissolved in 0.75 mL of dimethyl sulfoxide-d6 (DMSO-d6) with a 0.03% (v/v) TMS internal standard at a concentration of 20 mg/mL at a 4.5-6 pH range.

As used herein, “molecular weight analysis” “molecular weight distribution analysis” or “SEC-RID” or similar terms, refer to a method wherein samples were prepared by reconstituting dried powders into a 10 mg/mL solution in nano pure water. Samples were analyzed on an Agilent Infinity II 1260 RID coupled to an Agilent Infinity II 1260 HPLC. Separation was performed on an Agilent AdvanceBio SEC column (7.8 mm×300 mm, 2.7 μm particle size) with a 10 μL injection volume. Chromatographic solvents consisted of A: nano pure water and B: 95% acetonitrile in water (v/v) with a 50-minute gradient of 0.0-13.0 min, 0% B; 13.0-14.0 min, 30% B; 14.0-50.0 min 0% B. The flow rate was set to 1.00 mL/min and the column temperature was set at 35° C. The RID is operated in positive signal polarity mode and a 2.31 Hz peak width at 35° C. with 500,000 nRIU attenuation. Samples were integrated using Agilent ChemStation data analysis and molecular weights determined using the Agilent Cirrus GPC program along with a set of Dextran standards spanning the resolution of 180-150,000 Da. It will be appreciated by one having skill in the art that SEC-RID is subject to random, experimental error, and the molecular weights inferred therefrom should therefore be read to encompass reasonable variations from the stated value. Specifically, in some aspects, the molecular weights of oligosaccharide compositions described herein include variations of +20% of the stated molecular weight, or in some aspects, +10% of the stated molecular weight, or in some aspects, +5% of the stated molecular weight.

Unless otherwise specified, the term “average molecular weight” “average molecular mass” “mean molecular weight” “mean molecular mass” or similar terms refers to weight average molecular weight. Generally, unless otherwise specified, when an oligosaccharide or an oligosaccharide composition is described herein to have a specified average molecular weight (e.g., the molecular weight distribution of beta glucan oligosaccharides is such that at least 50% of the mass is smaller than 5 kDa), such values can be calculated with the aid of molecular weight analysis as described herein.

As used herein, the term “bioactive” or “bioactivity” refers to one or more of: decreasing glycemic response, decreasing Hba1c levels, inhibiting SGLT1 transport, inhibiting alpha-amylase, inhibiting alpha-glucosidase, increasing the production of GLP-1, enhancing microbial production of short chain fatty acids, enhancing microbial production of butyrate, enhancing microbial production of lactate, enhancing microbial production of acetate, increasing the members of the gut microbiota that produce short chain fatty acids, increasing the members of the gut microbiota that produce butyrate, increasing the members of the gut microbiota that produce lactate, increasing the members of the gut microbiota that produce acetate, increasing abundances of Clostridium butyricum in the gut, increasing abundances of Bifidobacterium in the gut, increasing abundances of Lactobacilii in the gut, or increasing abundances of Lactobacillus GG rhamnosus in the gut. In aspects where bioactivity refers to increasing or decreasing (e.g., increasing the production of GLP-1, decreasing Hba1c levels, etc.), the increase or decrease refers to a biologically relevant increase or biologically relevant decrease, respectfully, as defined herein.

As used herein, “enhances microbial production” or “enhancing microbial production” refers to a biologically relevant increase in production of a particular metabolite or group of metabolites. In some aspects, a biologically relevant increase is a statistically significant change as measured by parametric or non-parametric tests. In some aspects, a biologically relevant increase can be measured in feces, serum, urine, or organ tissue. In some aspects, a biologically relevant increase is measured through a host metabolic product (choline can be measured as TMA or TMAO). In some aspects, a biologically relevant increase is a 10% increase or a 100% increase or a 500% increase or a 1.000% increase or more. In some aspects, the biologically relevant increase can be in the absolute amount of a metabolite or group of metabolites. In some aspects, the biologically relevant increase can be the rate that a metabolite or group of metabolites are produced. In some aspects, the biologically relevant increase can be in the relative amount of a metabolite or group of metabolites.

As used herein, “decreases microbial production” refers to a biologically relevant decrease in the production of a particular metabolite or group of metabolites. In some aspects, a biologically relevant decrease is a statistically significant change as measured by parametric or non-parametric tests. In some aspects, a biologically relevant decrease can be measured in feces, serum, urine, or organ tissue. In some aspects, a biologically relevant decrease is measured through a host metabolic product (choline can be measured as TMA or TMAO). In some aspects, a biologically relevant decrease is a 10% decrease or a 20% decrease or a 50% decrease or a 75% decrease or a 90% decrease or more. In some aspects, the biologically relevant decrease can be in the absolute amount of a metabolite or group of metabolites. In some aspects, the biologically relevant decrease can be the rate that a metabolite or group of metabolites are produced. In some aspects, the biologically relevant decrease can be in the relative amount of a metabolite or group of metabolites.

As used herein, “decreases microbial utilization” refers to a biologically relevant increase in the amount of a particular metabolite or group of metabolites due to lower microbial utilization. In some aspects, a biologically relevant increase is a statistically significant change as measured by parametric or non-parametric tests. In some aspects, a biologically relevant increase can be measured in feces, serum, urine, or organ tissue. In some aspects, a biologically relevant increase is measured through a host metabolic product (choline can be measured as TMA or TMAO). In some aspects, a biologically relevant increase is a 10% increase or a 100% increase or a 500% increase or a 1,000% increase or more. In some aspects, the biologically relevant increase can be in the absolute amount of a metabolite or group of metabolites. In some aspects, the biologically relevant increase can be the rate that a metabolite or group of metabolites are produced. In some aspects, the biologically relevant increase can be in the relative amount of a metabolite or group of metabolites.

As used herein, the term “relative abundance of a bacteria” means the abundance of that bacteria relative to other bacteria in the microbiota in or on the particular organ of a complex organism, such as a human or mammal.

As used herein, “slows microbial utilization” refers to a biologically relevant increase in the amount or buildup of a particular metabolite or group of metabolites due to slowed microbial utilization. In some aspects, a biologically relevant increase is a statistically significant change as measured by parametric or non-parametric tests. In some aspects, a biologically relevant increase can be measured in feces, serum, urine, or organ tissue. In some aspects, a biologically relevant increase is measured through a host metabolic product (choline can be measured as TMA or TMAO). In some aspects, a biologically relevant increase is a 10% increase or a 100% increase or a 500% increase or a 1.000% increase or more. In some aspects, the biologically relevant increase can be in the absolute amount of a metabolite or group of metabolites. In some aspects, the biologically relevant increase can be the rate that a metabolite or group of metabolites are produced. In some aspects, the biologically relevant increase can be in the relative amount of a metabolite or group of metabolites.

As used herein, “increases abundance of” refers to a biologically relevant increase in the population of a certain bacterial taxa.

In some aspects, a “biologically relevant increase” is a statistically significant change as measured by parametric or non-parametric tests, generally in reference to the effects of a method comprising administering an oligosaccharide composition or formulation thereof to a subject, or in an in vitro context, relative to an otherwise identical method that does not include administering the oligosaccharide composition or formulation thereof. In some aspects, a biologically relevant increase can be measured in feces, jejunum, cecum, ileum, stomach, large intestines, duodenum, mouth, respiratory tract, skin, urogenital tract, vaginal tract, or other microbial community. In some aspects, a biologically relevant increase is a 10% increase or a 5× increase or a 10× increase or a 50× increase or a 100× or 1,000× increase or 10,000× or more. In some aspects the biologically relevant increase can be in the absolute amount of a taxa or group of taxa, or amount of a given species (e.g., short chain fatty acid, GLP-1, etc.). In some aspects, the biologically relevant increase can be the rate that a taxa, group of taxa, or other given species increases in the microbial community or in a subject (or location therein, such as a GI tract). In some aspects, the biologically relevant increase can be in the relative amount of a taxa, group of taxa, or other given species in a microbial community or in a subject (or location therein, such as a GI tract). In some aspects, an increase in abundance refers to the presence of one microbial taxa as compared to another microbial taxa, or one given species compare to another given species. “Biologically relevant decrease,” “biologically relevant change,” “biologically relevant amount.” and similar such terms can be similarly understood.

As used herein, “stimulates” in reference to a receptor means a given species (e.g., butyrate, propionate, etc.) acts as an agonist by binding to the receptor, which initiates an immune response.

As used herein, “increases the production” of a given species (e.g., a short chain fatty acids. GLP-1, etc.) means a biologically relevant increase in the production of the given species.

As used herein, “inhibits histone deacetylases” means the enzymatic activity of histone deacetylase is reduced by a biologically relevant amount or even eliminated.

As used herein, “increases the expression of” in reference to a gene (e.g., Muc2, occluding, claudin-4, ZO-1, etc.) means expression of the gene is increased by a biologically relevant amount.

As used herein, “lowers A1c levels” means a biologically relevant decrease in A1c levels.

As used herein, “lowers an inflammatory gastrointestinal marker” means a biologically relevant decrease in the amount of inflammatory gastrointestinal marker.

As used herein, “increases an anti-inflammatory gastrointestinal marker” means a biologically relevant increase in the amount of anti-inflammatory gastrointestinal marker.

As used herein, “lowers inflammation” in reference to a subject or GI tract of a subject means a decrease in one or more of TNF-α, IL1-β, IL-6, or other known inflammatory markers, or any combination thereof, when compared to the levels of the same markers in a subject that is not subjected to the relevant administering or treatment steps with a formulation or oligosaccharide composition described herein.

As used herein, “decreases intestinal barrier permeability” in reference to a subject or GI tract of a subject means (1) a decrease in TEER values, and/or (2) an increase in the expression of one or more of Muc2, occluding, claudin-4, ZO-1 genes, or and/or (3) other known intestinal barrier permeability markers, or (4) any combination thereof, when compared to the same markers in a subject that is not subjected to the relevant administering or treatment steps with a formulation or oligosaccharide composition described herein.

“Therapy” means treatment given or action taken to reduce or eliminate symptoms of a disease or pathological condition.

As used herein, a “therapeutically effective amount” or “effective amount” of the disclosed compounds (e.g., oligosaccharide, oligosaccharide composition, and/or synthetic composition) is a dosage of the compound that is sufficient to achieve a desired therapeutic or other outcome or effect, such as an anti-inflammatory effect, stimulation of growth of specified microbiota, and so forth. For example, a therapeutically effective amount of a compound may be such that the subject receives a dosage of about 0.1 μg/kg body weight/day to about 1000 mg/kg body weight/day, for example, a dosage of about 1 μg/kg body weight/day to about 1000 μg/kg body weight/day, such as a dosage of about 5 μg/kg body weight/day to about 500 μg/kg body weight/day. The compound(s) herein may administered in one or more doses, such as on a regular basis, including once-a-day, twice-a-day, every two days, weekly, or bi-weekly for a specified time period in order to achieve and/or maintain the desired therapeutic effect.

As used herein, “treatment” or “treat” refers to a therapeutic intervention that ameliorates a sign or symptom of a disease or pathological condition after it has begun to develop, and also includes addressing a medical condition or disease with the objective of improving or stabilizing an outcome in the subject being treated or addressing an underlying nutritional need. “Treatment” or “treat” therefore include the dietary or nutritional management of the medical condition or disease by addressing nutritional needs of the person being treated. “Treating.” “treat,” and “treatment” have grammatically corresponding meanings. As used herein, the term “ameliorating,” with reference to a disease or pathological condition, refers to any observable beneficial effect of the treatment. The beneficial effect can be evidenced, for example, by a delayed onset of clinical symptoms of the disease or condition in a susceptible subject, a reduction in severity of some or all clinical symptoms of the disease or condition, a slower progression of the disease or condition, an improvement in the overall health or well-being of the subject, or by other parameters well known in the art that are specific to the particular disease or condition. The phrases “treating a disease.” “treating a condition,” and similar terms are inclusive of inhibiting the full development of a disease or condition, for example, in a subject who is at risk for a disease or condition, or who has a disease or condition, such as inflammatory bowel disease (IBD), Crohn's disease, ulcerative colitis, bacterial vaginosis, cardiovascular disease, chronic kidney disease, a nervous system disorder, allergic reaction, atopic dermatitis, and so forth.

As used herein, “preventive treatment” or “prevention” means treatment given or action taken to diminish the risk of onset or recurrence of a disease. Preventing a disease or condition refers to prophylactically administering a composition to a subject who does not exhibit signs of a disease or condition, or exhibits only early signs of the disease or condition, for the purpose of decreasing the risk of developing a pathology or condition, or diminishing the severity of a pathology or condition. Although in some aspects the disease or condition is avoided, either permanently or subject to re-treatment, in alternative aspects the onset of the disease or condition is delayed. “Primary prevention” means prevention of the initial onset of a condition in an individual. “Secondary prevention” means, in a subject who has a condition or who has had a condition, (i) prevention of reoccurrence of the condition, (ii) increase in the duration of remission of the condition, and/or (iii) reduction in severity of symptoms of the condition. “Preventing,” “prevention,” “preventative treatment,” and “prevent” are used interchangeably.

“Emotional disorder” means a mental disorder involving a primary disturbance of emotions resulting in the emotions being distorted or inconsistent with circumstances. Emotional disorders include excessive anxiety, fear, anger, happiness, etc.

“Mood disorder” means a mental disorder involving a primary disturbance of a mood resulting in the mood being distorted or inconsistent with circumstances. Mood disorders include depression, major depressive disorder, dysthymia and bipolar disorder.

As used herein, the term “enteral administration” means any form for delivery of a composition to a subject that causes the deposition of the composition in the gastrointestinal tract (including the stomach). Methods of enteral administration include feeding through a naso-gastric tube or jejunum tube, oral, sublingual and rectal.

As used herein, “gastrointestinal tract” or “GI tract” means the passageway in the digestive system of a subject that includes all components from the esophagus to the anus (inclusive), as well as everything situated along the passageway including the stomach, intestines, and so forth. Generally, “gastrointestinal tract” is used interchangeably herein with the term “gut.”

As used herein, the term “microbiota”, “microflora” and “microbiome” mean a community of living microorganisms that typically inhabits a bodily organ or part, for example the gastro-intestinal or urogenital organs of complex organisms, such as mammals and humans. In particular, the most dominant members of the gastrointestinal microbiota include microorganisms of the phyla of Firmicutes, Bacteroidetes, Actinobacteria, Proteobacteria, Synergistetes, Verrucomicrobia, Fusobacteria, and Euryarchaeota; at genus level Bacteroides, Faecalibacterium, Bifidobacterium, Roseburia, Alistipes, Collinsella, Blautia, Coprococcus, Ruminococcus, Eubacterium and Dorea; at species level Bacteroides uniformis, Alistipes putredinis, Parabacteroides merdae, Ruminococcus bromii, Dorea longicatena, Bacteroides caccae, Bacteroides thetaiotaomicron, Eubacterium hallii, Ruminococcus torques, Faecalibacterium prausnitzii, Ruminococcus lactaris, Collinsella aerofaciens, Dorea formicigenerans, Bacteroides vulgatus and Roseburia intestinalis. The gastrointestinal microbiota includes the mucosa-associated microbiota, which is located in or attached to the mucous layer covering the epithelium of the gastrointestinal tract, and luminal-associated microbiota, which is found in the lumen of the gastrointestinal tract. Dominant members of the urogenital microbiota, for example, include Lactobacillus crispatus, Lactobacillus jensenii, Lactobacillus gasseri, Lactobacillus iners, and Lactobacillus vaginalis.

The term “Bifidobacterium” and its synonyms refer to a genus of anaerobic bacteria having beneficial properties for humans. Members of the Bifidobacterium genus are some of the major strains that make up the gut microbiome, the bacteria that reside in the gastrointestinal tract and have health benefits for their hosts (Guarner and Malagelada 2003).

As used herein, the terms “modulate,” “modulating,” or other similar terms refer to the ability of a disclosed compound (e.g., oligosaccharide or oligosaccharide composition) to alter the amount, degree, or rate of a biological function (including metabolite production), the progression of a disease, or amelioration of a condition. For example, modulating can refer to the ability of a compound to increase or decrease the abundance of a microorganism, increase or decrease production of a metabolite, or elicit a decrease in the inflammation, pain, incidence, or severity of a symptom associated with a particular condition or disease (e.g., associated with the gastrointestinal system, cardiovascular system, renal system, nervous system, immune system, and/or urogenital system).

As used herein, the term “modulation of microbiota” means exerting a modifying or controlling influence on microbiota, for example, an influence leading to an increase in the indigenous intestinal abundance of one or more types of microorganism, such as Bifidobacterium, and/or a metabolite producing bacteria, such as those that produce butyrate. In another example, the influence may lead to a reduction of the intestinal abundance of one or more types of microorganisms, such as Ruminococcus gnavus and/or Proteobacteria.

As used herein, the term “oral administration” means any form for the delivery of a composition to a subject through the mouth. Accordingly, oral administration is a form of enteral administration.

As used herein, the term “EP or Ethanol Precipitate” means a composition which is artificially prepared via the selective precipitation by the addition of a known concentration of ethanol and the separation of the non-soluble portion.

As used herein, the term “ES or Ethanol Supernatant” means a composition which is artificially prepared via the selective precipitation by the addition of a known concentration of ethanol and the separation of the soluble portion.

Disclosed herein are examples of starting materials (sometimes referred to herein as “one or more materials,” “materials,” “parent,” “parent polysaccharides,” “precursor polysaccharides” or similar terms) from which certain oligosaccharides and compositions thereof are derived. It will be appreciated by one having skill in the art that any toxic portions of said starting materials will be minimized or excluded from the methods, compositions, medicaments, and formulations herein.

As used herein, “arabinan” is a polysaccharide with a glycosidic linkage composition comprising at least 75% arabinose sub-units comprised of α-1,3, α-1,5, and α-1,3,5 linkages. In some aspects, arabinan contains 10-20% galactose subunits, 10-20% xylose subunits, or 10-20% of a combination of galactose and xylose subunits. In some aspects arabinan is “de-branched” and contains only α-1,5 arabinose linkages.

As used herein, “arabinan oligosaccharide” means an oligosaccharide that contains alpha-linked arabinan residues and that optionally resembles a beetroot arabinan and/or a legume arabinan, such as, a pea arabinan, and/or a soy arabinan. In some aspects, the oligosaccharide comprises alpha 1-5, alpha 1-3, or alpha 1-2 glycosidic linkages. Arabinan oligosaccharides can be linear or branched. In some aspects, the molecular weight distribution of arabinan oligosaccharides is such that at least 50% of the mass is smaller than 50 kDa. Arabinan oligosaccharides can be made through Fenton-type depolymerizations as described in WO2021097138A1, WO2018236917A1, WO2020247389A1, and WO2022241163A1, which are each incorporated by reference herein in its entirety, and more specifically for methodologies of synthesis, to the extent not inconsistent with the description herein.

As used herein, “barley” comprises a polysaccharide comprising beta glucan with a glycosidic linkage composition comprising a glucose backbone comprising β-1,4 and β-1,3 in about a 4 to 1 ratio.

As used herein, “curdlan” is a polysaccharide with a glycosidic linkage composition comprising a β-1,3 glucose backbone.

As used herein, “glucomannan” is a polysaccharide with a glycosidic linkage composition of about 60% β-1,4 mannose and about 40% β-1,4 glucose backbone.

As used herein, “xylan” is a polysaccharide with a glycosidic linkage composition comprising a β-1,4 xy lose backbone with about 13% α-1,2 Glucose-4-OMe.

As used herein, “arabinogalactan” is a polysaccharide with a glycosidic linkage composition comprising a β-1,4 xylose backbone with α-1,3 and α-1,2 arabinose branches in about a 1 to 2 ratio.

As used herein, “locust bean gum” is a polysaccharide with a glycosidic linkage composition of about 73% β-1,4 mannose backbone, with about 23% decorated with β-1,4 galactose.

As used herein, “galactan” is a polysaccharide with a glycosidic linkage composition comprising a β-1,4 galactan backbone.

As used herein, “legume” refers to any part of a plant in the family Fabaceae (or Leguminosae), or the fruit or seed of such a plant. Examples of legumes include peas, beans, soy, chickpeas, peanuts, lentils, lupins, mesquite, carob, tamarind, alfalfa, and clover. In some aspects, legume refers to by-products of the plant during harvest or food processing. Non-limiting examples include powders, pods, flowers, stems, roots, seeds, fiber, or crude protein. Legume may refer to the solid material after roasting, fermentation, hot-water, enzymatic, chemical, alkaline, super critical fluid, sun drying, organic solvent, acidic, mechanical pressure or pressure-based extractions.

As used herein, “lichenan” is a polysaccharide (comprising beta glucan) with a glycosidic linkage composition comprising a β-1,4 glucose backbone with alternating β-1,3 glucose about 33% of the time.

As used herein, “galactomannan” is a polysaccharide with a glycosidic linkage composition comprising a β-1,4 mannose backbone, with about 22% α-1,3 galactose branching.

As used herein, “β-glucan” (also called “beta-glucan”) is a polysaccharide with a glycosidic linkage composition comprising a glucose backbone comprising β-1,4 and β-1,3 in about a 4 to 1 ratio. In embodiments, beta-glucan has weight average molecular weight (Mw) of 500 kDa or greater.

As used herein, “xyloglucan” is a polysaccharide with a glycosidic linkage composition comprising a β-1,4 glucose backbone with α-1,6 xylose branches

As used herein, “arabinoxylan” is a polysaccharide with a glycosidic linkage composition comprising β-1,4 xylose backbone with α-1,3 and α-1,2 arabinose branches in about a 1 to 2 ratio.

As used herein, “olive” refers to any part of the plant in the genus Olea. “Olive” may refer to Olea europaea, Olea cuspidate, Olea oleaster, Olea cerasiformis (maderensis), Olea guanchica, Olea laperrinei, Olea maroccana, Olea Canarium, or other species. “Olive” may refer but not limited to colors of green, shades of red, brown, or black. “Olive” may refer to by-products of the plant during harvest or food processing, non-limiting examples include olive flowers and their associated parts (Stigma, style, filament, pedals, flora axis, articulation and nectary), Olive ovules, Olive oil, Olive oil press cake, Olive mill waste water, Olive pomace, olive composts or Olive sludges “Olive” may refer to the solid material after roasting, fermentation, hot-water, enzymatic, chemical, alkaline, super critical fluid, organic solvent, acidic, mechanical pressure or pressure based extractions. “olive” may refer to other non-Olea genus, which are colloquially known as Olive.

As used herein, “Ulvaceae (Ulva intestinalis)” refers to any part of the plant in the genus Ulva, Enteronia, Gemina, Letterstedtia, Lobata, Ochlochaete, Percursaria, Phycoseris, Ruthnielsenia, Solenia, Ulvaria, Umbraulva or Enteromorpha. “Ulvaceae Ulva” may refer to Ulva intestinalis, Ulva lactuca or other species. “Ulvaceae ulva” may refer to by-products of the plant during harvest or food processing, non-limiting examples include a flat or a hollow tubular thallus, Ulvaceae ulva leafs, frond or blades, Ulvaceae ulva stripe or Ulvaceae ulva hold fast. “Ulvaceae ulva” may refer to the solid material after roasting, fermentation, hot-water, enzymatic, chemical, alkaline, super critical fluid, sun drying, organic solvent, acidic, mechanical pressure or pressure based extractions. “Ulvaceae ulva” may refer to other non-ulva genus, which are colloquially known as Sea lettuce, gutweed or grass kelp.

As used herein, “Macrocystis pyrifera” refers to any part of the plant in the genus Macrocystis. “Macrocystis pyrifera” may refer to Fucus pyrifer L., Laminaria pyrifera (L.) Lamouroux, Macrocystis humboldtii (Bonpland) C.Ag., Macrocystis planicaulis C. Agardh, Macrocystis pyrifera var. humboldtii, or other species. “Macrocystis pyrifera” may refer to by-products of the plant during harvest or food processing, non-limiting examples include a flat or a hollow tubular thallus, Macrocystis pyrifera blades, Macrocystis pyrifera air bladders (Pneumatocyst), Macrocystis pyrifera stripe, Macrocystis pyrifera sporophylls or Macrocystis pyrifera hold fast. “Macrocystis pyrifera” may refer to the solid material after roasting, fermentation, hot-water, enzymatic, chemical, alkaline, super critical fluid, sun drying, organic solvent, acidic, mechanical pressure or pressure based extractions. “Macrocystis pyrifera” may refer to other non-Macrocystis genus, which are colloquially known as giant kelp, giant bladder kelp, pacific kelp, or large brown algae.

As used herein, “sugar cane” refers to any part of the plant in the genus Saccharum. “Sugar cane” may refer to Saccharum officinarum, Saccharum sinense, Saccharum barberi, Saccharum arundinaceum, Saccharum bengalense, Saccharum edule, Saccharum procerum, Saccharum ravennae, Saccharum robustum, Saccharum spontaneum, hybrids of two, three or more species, or other species. In some aspects, “sugar cane” refers to by-products of the plant during harvest or food processing, non-limiting examples include, sugar cane leaf (barbojo), sugar cane stalks (canc), raw sugarcane cylinders or cubes, sugar cane bagasse, fresh sugar cane juice, Sugar cane molasses, sugar cane rapadura, sugar cane flour or processed sugar cane. In some aspects, sugar cane refers to the solid material after roasting, fermentation, hot-water, enzymatic, chemical, alkaline, super critical fluid, sun drying, organic solvent, acidic, mechanical pressure or pressure based extractions. Sugar cane may also refer to “Power Cane”. In some aspects, sugar cane refers to other non-Saccharum genus, which are colloquially known as sugar cane.

As used herein, “Carrot” refers to any part of the plant in the genus Daucus. “Carrot” may refer to Daucus carota, Daucus sativus, Carota sativa, or other species. “Carrot” may refer to by-products of the plant during harvest or food processing, non-limiting examples include, carrot flower, carrot stem, carrot seed, carrot leaf, carrot tap root or carrot lateral roots. “Carrot” may refer to the solid material after roasting, fermentation, hot-water, enzymatic, chemical, alkaline, super critical fluid, sun drying, organic solvent, acidic, mechanical pressure or pressure based extractions. “Carrot” may refer to other non-Daucus genus, which are colloquially known as Daucus carota subsp. Sativus, or wild carrot.

As used herein, “soy” refers to any part of the plant in the genus Glycine or Soja. Soy may refer to Dolichos soja L., Glycine angustifolia Miq., Glycine gracilis Skvortsov, Glycine hispida (Moench) Maxim., Glycine soja, Phaseolus max L., Soja angustifolia, Soja hispida Moench, Soja japonica savi, Soja max, Soja H., Soja viridis or other species. In some aspects, soy refers to by-products of the plant during harvest or food processing, non-limiting examples include, soy root, soy stem, soy leaves, soy flowers, soy fruiting pods, soy bean, soy protein, soy okra (pulp or curd), soy fiber or soy bean testa. In some aspects, soy refers to the solid material after roasting, fermentation, hot-water, enzymatic, chemical, alkaline, super critical fluid, sun drying, organic solvent, acidic, mechanical pressure or pressure based extractions. “Soy” may refer to other non-Glycine or soja genus, which are colloquially known as soy bean, kongbiji or soya.

As used herein, “Sphingomonas elodea extract” refers to any part of the bacteria in the genus Sphingomonas. “Sphingomonas elodea extract” may refer to Pseudomonas elodea or other species. “Sphingomonas elodea” may refer to by-products of the bacteria during harvest or food processing, non-limiting examples include, extracellular polysaccharides, intracellular polysaccharides, Sphingomonas elodea cell wall, Sphingomonas elodea carbohydrate membrane, or purified Sphingomonas elodea gellan gum polysaccharides. “Sphingomonas elodea extract” may refer to the solid material after roasting, fermentation, hot-water, enzymatic, chemical, alkaline, super critical fluid, sun drying, organic solvent, acidic, mechanical pressure or pressure based extractions. “Sphingomonas elodea extract” may refer to other non-Sphingomonas which are colloquially known as gellan gum, bacteria extract or gelling agent.

As used herein, “coffee” refers to any part of the plant in the genus Coffea. “Coffee” may refer to Coffea arabica, Coffea, robusta, Coffea liberica, or other species. In some aspects, coffee refers to by-products of the plant during harvest or food processing, non-limiting examples include spent coffee grounds, coffee extracts, coffee beans, coffee parchment coffee pulp, coffee berries, coffee cherries, coffee husk, coffee silver skin, coffee pectin layer, coffee bean outer skin, coffee hulls, coffee leaves, coffee roots, coffee stems, or coffee leaves. In some aspects, coffee refers to the solid material after roasting, fermentation, hot-water, enzymatic, chemical, alkaline, super critical fluid, organic solvent, acidic, mechanical pressure or pressure based extractions. “Coffee” may refer to other non Coffea genus, which are colloquially known as coffee.

As used herein, “Xanthomonas campestris extract” refers to any part of the bacteria in the genus Xanthomonas. “Xanthomonas campestris extract” may refer to extracts from Xanthomonas campestris pv. armoraciae, Xanthomonas campestris pv. begoniae A, Xanthomonas campestris pv. begoniae B, Xanthomonas campestris pv. campestris, Xanthomonas campestris pv. cannabis, Xanthomonas campestris pv. carota, Xanthomonas campestris pv. corylina, Xanthomonas campestris pv. dieffenbachiae, Xanthomonas campestris pv. glycines syn. Xanthomonas axonopodis pv. glycines, Xanthomonas campestris pv. graminis, Xanthomonas campestris pv. hederae, Xanthomonas campestris pv. hyacinthi, Xanthomonas campestris pv. juglandis, Xanthomonas campestris pv. malvacearum or Xanthomonas citri subsp. malvacearum, Xanthomonas campestris pv. musacearum, Xanthomonas campestris pv. mangiferaeindicae, Xanthomonas campestris pv. mori, Xanthomonas campestris pv. nigromaculans, Xanthomonas campestris pv. pelargonii, Xanthomonas campestris pv. phaseoli, Xanthomonas campestris pv. poinsettiicola, Xanthomonas campestris pv. pruni, Xanthomonas campestris pv. raphani, Xanthomonas campestris pv. sesami, Xanthomonas campestris pv. tardicrescens, Xanthomonas campestris pv. translucens, Xanthomonas campestris pv. vesicatoria, Xanthomonas campestris pv. viticola or other species. In some aspects, Xanthomonas campestris extract refers to by-products of the bacteria during harvest or food processing, non-limiting examples include. Xanthomonas campestris extracellular polysaccharides, Xanthomonas campestris intracellular polysaccharides. Xanthomonas campestris cell wall. Xanthomonas campestris carbohydrate membrane, or purified Xanthomonas campestris xanthan gum polysaccharides. In some aspects, Xanthomonas campestris extract refers to the solid material after roasting, fermentation, hot-water, enzymatic, chemical, alkaline, super critical fluid, sun drying, organic solvent, acidic, mechanical pressure or pressure based extractions. “Xanthomonas campestris extract” may refer to other non-Xanthomonas which are colloquially known as xanthan gum, bacteria extract or gelling agent.

As used herein, “xanthan gum” refers to a polysaccharide with a β-1,4 glucose backbone with alternating α-1,2 mannose. Xanthan gum can be derived from Xanthomonas campestris. In some aspects, the oligosaccharides derived from xanthan gum (“xanthan gum oligosaccharide”) possess various structural features similar to those in the parent polysaccharide. In some aspects, xanthan gum oligosaccharides comprise a composition having 63.86% glucose and 30.64% mannose. In some aspects, xanthan gum oligosaccharides comprise a glycosidic linkage composition of 29.88% 4-glucose, 16.02% 2-mannose, 12.24% 3,6-galactose, 11.54% terminal glucose and 6.09% 4,6-mannose. In some aspects, xanthan gum oligosaccharides are created through enzymatic, chemical, or biological synthesis or through the depolymerization of beta glucans via enzymatic, chemical, physical, or biological processes. In some aspects, xanthan gum oligosaccharides are made through Fenton-type depolymerizations as described in WO2021097138A1, WO2018236917A1, WO2020247389A1, and WO2022241163A1, which are each incorporated by reference herein it its entirety, specifically for methodologies of synthesis, to the extent not inconsistent with the description herein. Oligosaccharide CLX123 provides examples of xanthan gum oligosaccharides.

As used herein, “pea” refers to any part of the plant in the genus Pisum, Cajanus, lathyrus or Vigina. In some aspects, pea refers to Pisum sativum, Cajanus cajanor, Vigna unguiculata, Lathyrus aphaca or other species. In some aspects, pea refers to by-products of the plant during harvest or food processing, non-limiting examples include pea powder, pea pods, pea flower, pea stem, pea stipules, pea root, pea seeds, pea fiber, or crude pea protein. In some aspects, pea refers to the solid material after roasting, fermentation, hot-water, enzymatic, chemical, alkaline, super critical fluid, sun drying, organic solvent, acidic, mechanical pressure or pressure based extractions. Pea may refer to other non Pisum, Cajanus, lathyrus or Vigina genus, which are colloquially known as pea, snow pea, split pea, snap pea, field pea or sugar pea.

As used herein, “tomato” refers to any part of the plant in the genus Solanum. “Tomato” may refer to Solanum lycopersicum, Lycopersicon lycopersicum, Lycopersicon esculentum or other species. “Tomato” may refer to by-products of the plant during harvest or food processing, non-limiting examples include tomato peels, tomato berries, tomato stalks, tomato flowers, tomato seeds, tomato berry flesh, or tomato root. “Tomato” may refer to the solid material after roasting, fermentation, hot-water, enzymatic, chemical, alkaline, super critical fluid, sun drying, organic solvent, acidic, mechanical pressure or pressure based extractions. “Tomato” may refer to other non-Lupinus Solanum, which are colloquially known as tomatoes.

As used herein, “Saccharomyces cerevisiae” refers to any part of the yeast in the genus Saccharomyces. In some aspects, Saccharomyces cerevisiae refers to Saccharomyces cerevisiae or other species. Saccharomyces cerevisiae may refer to by-products of the yeast cell during harvest or food processing, non-limiting examples include yeast cell membrane, yeast growth media, yeast extracellular polysaccharides, yeast intracellular polysaccharides, yeast cell extracts, yeast fiber, yeast polysaccharides, mannose rich yeast extracts, or yeast spores. In some aspects, Saccharomyces cerevisiae refers to the solid material after roasting, fermentation, hot-water, enzymatic, chemical, alkaline, super critical fluid, sun drying, organic solvent, acidic, mechanical pressure or pressure based extractions. In some aspects, Saccharomyces cerevisiae may refer to other non-Saccharomyces genus, which are colloquially known as baker yeast.

As used herein, “yeast beta glucan” refers to a beta glucan found in the cell walls of yeast. In some aspects, yeast beta glucan refers to a polysaccharide containing beta-linked glucose units that may be in the beta-3 position, the beta-4 position, or the beta-6 position. In some aspects, yeast beta glucan is found alongside other polymers such as mannans. In some aspects, yeast beta glucan refers to a structure, wherein, the backbone is beta-3 linked and the beta-6 linkages are long branches. In some aspects, yeast beta glucan is derived from Saccharomyces cerevisiae or other yeast within or outside of the Saccharomyces genus. In some aspects, yeast beta glucan refers to the solid material after roasting, fermentation, hot-water, enzymatic, chemical, alkaline, super critical fluid, sun drying, organic solvent, acidic, mechanical pressure or pressure based extractions.

As used herein, “beta glucan” (also called “β-glucan”) is a polysaccharide that contains β-linked glucose residues. In some embodiments, beta glucan refers to a collective set of polysaccharides that encompass cereal beta glucans, yeast beta glucans, and fungal beta glucans. In some aspects, beta glucan polymers comprise β1-3, β1-4, or β1-6 glycosidic linkages. In some aspects, beta glucan polymers comprises a glycosidic linkage composition comprising a glucose backbone comprising β-1,4 and β-1,3 in about a 4 to 1 ratio. In some aspects, beta glucans are linear or branched. In some aspects, within each class of beta glucans, the distribution of polymers is such that at least 80% of the mass is larger than 50 kDa. In some aspects, “beta glucan” refers to the solid material after roasting, fermentation, hot-water, enzymatic, chemical, alkaline, super critical fluid, sun drying, organic solvent, acidic, mechanical pressure or pressure based extractions. CLX115-PS is one example of a beta glucan.

As used herein, “beta glucan oligosaccharide” (also called “β-glucan oligosaccharide”) refers to a collective set of oligosaccharides that resemble a cereal beta glucan, a yeast beta glucan, and/or a fungal beta glucan. In aspects, beta glucan oligosaccharides are classes of oligosaccharides that contain exclusively β-linked glucose residues. In some aspects, the beta glucan oligosaccharide comprises β1-3, β1-4, or β1-6 glycosidic linkages. In some aspects, beta glucan oligosaccharides are linear or branched. In some aspects, the molecular weight distribution of beta glucan oligosaccharides is such that at least 50% of the mass is smaller than 5 kDa. In some aspects, “beta glucan oligosaccharides” refers to oligosaccharides created through enzymatic, chemical, or biological synthesis or through the depolymerization of beta glucans via enzymatic, chemical, physical, or biological processes. Beta glucan oligosaccharides may be made through Fenton-type depolymerizations as described in WO2021097138A1, WO2018236917A1, and WO2020247389A1, which are each incorporated by reference herein in its entirety, and more specifically for methodologies of synthesis, to the extent not inconsistent with the description herein. Oligosaccharide compositions CLX112, CLX115, and CLX115Cu are all examples of beta glucan oligosaccharides.

As used herein, “cereal beta glucan” refers to a beta glucan found in the cell walls of cereals. “Cereal beta glucan” refers to a polysaccharide containing beta-linked glucose units are in the beta-3 position and beta-4 positions. In some aspects, cereal beta glucan is found alongside other polymers such as cellulose, starch, and arabinoxylans. In some embodiments, “cereal beta glucan” refers to a structure, wherein, the linear polymer is comprised of beta-4 linked glucose residues with beta-3 linked residues interspersed at a ratio of about 1:1 to 5:1 beta-4: beta-3 linked glucose residues. In some embodiments, cereal beta glucan has a structure in which the linear polymer is comprised of beta-4 linked glucose residues with beta-3 linked residues interspersed at a ratio of about 3:1 to 5:1 beta-4: beta-3 linked glucose residues. Cereal beta glucan can be derived from cereals and grains such as oats, barley, wheat, rye, and rice. Beta glucans can come from other cereals and grains. Beta glucans can be extracted from the bran or the endosperm of cereals and grains. In some aspects, cercal beta glucan refers to the solid material after roasting, fermentation, hot-water, enzymatic, chemical, alkaline, super critical fluid, sun drying, organic solvent, acidic, mechanical pressure or pressure based extractions. In some embodiments, the distribution of polymers in cereal beta glucan is such that at least 80% of the mass is larger than 50 kDa. CLX115-PS is an example of a cereal beta glucan.

As used herein, “cereal beta glucan oligosaccharides” refers to an oligosaccharide that resembles beta glucan found in the cell walls of cereals and contains beta-linked glucose units that are in the beta-3 position and beta-4 positions. In some embodiments, cereal beta glucan oligosaccharides are found alongside polysaccharides or oligosaccharides such as cellulose, starch, and arabinoxylans in their polysaccharide or oligosaccharide forms. In some aspects, cereal beta glucan oligosaccharides refers to a structure, wherein, the linear polymer is comprised of beta-4 linked glucose residues with beta-3 linked residues interspersed at a ratio of about 1:1 to 5:1 beta-4: beta-3 linked glucose residues. In some aspects, cereal beta glucan oligosaccharides have a structure in which the linear polymer is comprised of beta-4 linked glucose residues with beta-3 linked residues interspersed at a ratio of about 3:1 to 5:1 beta-4: beta-3 linked glucose residues. Cereal beta glucan oligosaccharides can be derived from cereal beta glucans from cereals and grains such as oats, barley, wheat, rye, and rice. Cereal beta glucan oligosaccharides can come from other cereal and grain beta glucans. Cereal beta glucan oligosaccharides can come from beta glucans extracted from the bran or the endosperm of cereals and grains. Cereal beta glucan oligosaccharides can refer to the solid material after roasting, fermentation, hot-water, enzymatic, chemical, alkaline, super critical fluid, sun drying, organic solvent, acidic, mechanical pressure or pressure based extractions. In some aspects, the molecular weight distribution of cereal beta glucan oligosaccharides is such that at least 50% of the mass is smaller than 5 kDa. In some aspects, cereal beta glucan oligosaccharides refers to oligosaccharides created through enzymatic, chemical, or biological synthesis or through the depolymerization of beta glucans via enzymatic, chemical, physical, or biological processes. In some aspects, cereal beta glucan oligosaccharides are made through Fenton-type depolymerizations as described in WO2021097138A1, WO2018236917A1, WO2020247389A1. Oligosaccharides CLX112, CLX115, and CLX115Cu are all examples of cereal beta glucan oligosaccharides.

As used herein, “Tragacanth gum” refers to any part of the yeast in the genus Astragalus. “Tragacanth gum” may refer to Astragalus adscendens, Astragalus gummifer, Astragalus brachycalyx, and Astragalus tragacantha or other species. Tragacanth gum may refer to by-products of the tragacanth plant during harvest or food processing, non-limiting examples include Tragacanth sap, Tragacanth powder, Tragacanth beans, Tragacanth leafs or Tragacanth bark. “Tragacanth gum” may refer to the solid material after roasting, fermentation, hot-water, enzymatic, chemical, alkaline, super critical fluid, sun drying, organic solvent, acidic, mechanical pressure or pressure based extractions. Tragacanth gum may refer to other non-Astragalus genus, which are colloquially known as Shiraz gum, shiraz, gum elect, Gond Kateera, or gum dragon.

As used herein, “orange” refers to any part of the plant in the genus Citrus. “Orange” may refer to Citrus maxima, Citrus reticulata, Citrus sinensis, Citrus aurantium, Citrus bergamia risso, Citrus trifoliata or other distinct species, varieties and hybrids. Orange may refer to by-products of the plant during harvest or food processing, non-limiting examples include Orange Rind, Orange pith Orange Pulp, Orange fiber, Orange Juice, Orange seeds, Orange leaves, Orange bark, or Orange flowers. In some aspects, orange refers to the solid material after roasting, fermentation, hot-water, enzymatic, chemical, alkaline, super critical fluid, sun drying, organic solvent, acidic, mechanical pressure or pressure based extractions. “Orange” may refer to other non-Citrus genus, which are colloquially known as sweet orange, bitter orange, Bergamot orange, Trifoliate orange or mandarin orange.

As used herein, “beet” refers to any part of the plant in the genus Beta. “Beet” may refer to Beta vulgarisor or other distinct species and subspecies, Adanesisi, Maritima, Vulgaris, Altissima, circla, flavescens, conditiva and crassa. “Beet” may refer to by-products of the plant during harvest or food processing, non-limiting examples include beet tap roots, beet stems, beet leaves, beet powder, beet fiber, and beetroots. “Beet” may refer to the solid material after roasting, fermentation, hot-water, enzymatic, chemical, alkaline, super critical fluid, sun drying, organic solvent, acidic, mechanical pressure, candied, pickling or pressure based extractions. “Beet” may refer to other non-beta genus, which are colloquially known as sugar beets, sea beets, spinach beets, Swiss chard, beet root, table beets, garden beet, red beet, dinner beat golden beet or mangel-wurzel.

As used herein, “Baobob” refers to any part of the plant in the genus Adansonia. “Baobob” may refer to Adansonia digitata, Adansonia grandidieri, Adansonia gregorii, Adansonia madagascariensis, Adansonia perrieri, Adansonia rubrostipa, Adansonia suarezensis, Adansonia za or other distinct species. “Baobob” may refer to by-products of the plant during harvest or food processing, non-limiting examples include Baobob Fruit, Baobob powder, Baobob bark, Baobob leaves, Baobob fiber, Baobob seeds, Baobob fruit pith, or Baobob flowers. “Baobob” may refer to the solid material after roasting, fermentation, hot-water, enzymatic, chemical, alkaline, super critical fluid, sun drying, organic solvent, acidic, mechanical pressure, or pressure based extractions. “Baobob” may refer to other non-Adansonia genus, which are colloquially known as boab, bottle tree, dead rat tree, monkey-bread tree or montane.

As used herein, “Karaya gum” refers to any part of the plant in the genus Sterculia. “Karaya gum” may refer to Sterculia urens, Cavallium urens, Clompanus urens, Kavalama urens or other distinct species. “Karaya gum” may refer to by-products of the plant during harvest or food processing, non-limiting examples include Karaya sap, Karaya powder, Karaya leaves, or Sterculia urens bark. “Karaya gum” may refer to the solid material after roasting, fermentation, hot-water, enzymatic, chemical, alkaline, super critical fluid, sun drying, organic solvent, acidic, mechanical pressure, or pressure based extractions. “Karaya gum” may refer to other non-Sterculia genus, which are colloquially known as Indian tragacanth gum, katira, kulu or gum Sterculia.

As used herein, “lupin galactan” refers to any part of the plant in the genus Lupinous. “Lupin” may refer to Lupinus arboreus, Lupinus hirsutus, Lupinus chamissonis, Lupinus albifrons, Lupinus excubitus, Lupinous albus, Lupinous mutabilis, Lupinus longifolius, Lupinous angustifolius or other distinct species. “Lupin Galactan” may refer to by-products of the plant during harvest or food processing, non-limiting examples include Lupin Bean, Lupin powder, Lupin seed, Lupin flower, lupin stem, “protein extracted” lupin, Lupin fiber, Defatted lupin flour. “Lupin Galactan” may refer to the solid material after roasting, fermentation, hot-water, enzymatic, chemical, alkaline, super critical fluid, sun drying, organic solvent, acidic, mechanical pressure, or pressure-based extractions. “Lupin” may refer to other non-Lupinus genus, which are colloquially known as lupin beans, white lupin, tarwi, chocho, kirku, turmus or blue lupin

As used herein, when a table, spectrum, or other data is referred to as representing the features or properties possessed by a particular composition, oligosaccharide, or other compound or mixture, unless specified otherwise, the same analysis method and procedure used to obtain the table, spectrum, or other data is to be used to determine the properties of the particular composition, oligosaccharide, or other compound or mixture.

Various compositions disclosed herein are identified by a “CLX” designation. CLX generally refers to an oligosaccharide composition. In embodiments where CLX is followed by “PS,” the CLX #-PS refers to a polysaccharide composition. Such CLX compositions can be prepared in any suitable manner and by any suitable method, including ground up synthetic methods (e.g., oligomerizing monomeric or shorter chain oligosaccharides into the indicated oligosaccharides), or by depolymerization methods (e.g., by depolymerizing polysaccharides or longer chain oligosaccharides into shorter chain oligosaccharides). For example, in some aspects, the CLX compositions disclosed herein can be prepared by a depolymerization method disclosed in WO 2018/236917 (Amicucci et al., “Production of bioactive oligosaccharides”) or WO 2021/097138 (Amicucci et al., “High-yield peroxide quench-controlled polysaccharide depolymerization and compositions thereof”), both of which are hereby incorporated by reference in their entireties for all purposes, and more specifically for methodologies of preparation, to the extent not inconsistent with the description herein. By way of example, such CLX compositions disclosed herein can be prepared by a method comprising dissolving the indicated source polysaccharide(s) (e.g., microbial curdlan, lichenan, xylan, etc.) in 20 ml of HPLC grade water in a capped reaction vessel and placed in a shaker-incubator for 20 min at 55° C. and 85 RPM. The pH of the solution is adjusted to 5.2, or about 5.2. Hydrogen peroxide (5 ml) and iron (II) sulfate or copper (II) sulfate (in either case, 2.75 mg in 50 μL water) are added to the reaction mixture and mixed thoroughly. The reaction in the capped reaction vessel is allowed to proceed in the shaker-incubator at 55° C. and 65 RPM for two hours. The capped reaction is cooled to 12° C. in a −20° C. freezer. Ammonium hydroxide (1 ml of 28% v/v to pH 8-12, such as pH 10.2) is used to adjust pH and sample is reacted at 45° C. in a shaker-incubator for 1 hour at 20 RPM, the cap is left loose to allow oxygen, ammonia, and carbon dioxide gases to be released. Alternatively, sodium hydroxide (65 μL, 10.45 M NaOH to pH 8-12, such as pH 10) may be used in place of ammonium hydroxide. The sample is then frozen and lyophilized, then stored at −80° C. The freeze-dried oligosaccharide mixture is rehydrated with the minimum amount of water required to allow for a free-flowing solution. This solution is then loaded onto a column containing 15 mL mixed bed ion exchange resin per gram (dry weight) of crude material, and the runoff is collected in a plastic freezer bag. Once the material is loaded onto the column, the column is then rinsed with 3 bed volumes of water. Finally, the runoff is sealed and frozen in the bag, then carefully shattered and subjected to lyophilization. In embodiments, the depolymerization method is performed using well-established production methods, such as batch processing or continuous processing.

For NMR analysis of the CLX compositions described herein, oligosaccharides were dissolved in D20 or D6-DMSO at a concentration of 50 mg/ml and were analyzed on a 600 MHz Bruker NMR spectrometer for their HSQC spectra.

The oligosaccharide compositions disclosed herein, including CLX compositions, are characterized, in part, by the relative amounts of monosaccharide subunits present in each composition. The amount of each subunit is represented as a percentage as defined herein under “hydrolytic monosaccharide compositional analysis” and/or as a “monosaccharide ratio” as defined herein. It will be appreciated by one having skill in the art that hydrolytic monosaccharide compositional analysis is subject to random, experimental error, and the percentages and ratios should therefore be read to encompass reasonable variations from the stated value. Specifically, in some aspects, percentages and ratios associated with monosaccharide subunits of oligosaccharide compositions include variations of +20% of the stated percentage or ratio. In keeping with this aspect, for a CLX composition recited as having 50% of its mass comprising glucose, it will be appreciated by a skilled artisan that said CLX composition may have anywhere between 40%-60% of its mass comprising glucose. As a further example of this aspect, a glucose: galactose ratio of 2:1 includes variations glucose: galactose ratios of 1.6:1 to 2.4:1 (e.g., 1.6:1, 1.8:1, 2:1, 2.2:1, 2.4:1) and 2:0.8 to 2:1.2 (e.g., 2:0.8, 2:0.9, 2:1, 2:1.1, 2:1.2). In some aspects, percentages and ratios associated with monosaccharide subunits of oligosaccharide compositions include variations of +10% of the stated percentage or ratio. In some aspects, percentages and ratios associated with monosaccharide subunits of oligosaccharide compositions include variations of +5% of the stated percentage or ratio. In some aspects, percentages and ratios associated with monosaccharide subunits of oligosaccharide compositions include variations of +1% of the stated percentage or ratio.

Relatedly, the oligosaccharide compositions, including CLX compositions, are also characterized by the relative amounts of glycosidic linkages present in each composition. The amount of each linkage is represented as a percentage as defined herein under “glycosidic linkage composition” and/or as a “linkage ratio” as defined herein. It will appreciated by one having skill in the art that glycosidic linkage composition analysis is subject to random, experimental error, and the percentages and ratios should therefore be read to encompass reasonable variations from the stated value. Specifically, in some aspects, percentages and ratios associated with glycosidic linkages of oligosaccharide compositions include variations of ±20% of the stated percentage or ratio. In some aspects, percentages and ratios associated with glycosidic linkages of oligosaccharide compositions include variations of ±10% of the stated percentage or ratio. In some aspects, percentages and ratios associated with glycosidic linkages of oligosaccharide compositions include variations of ±5% of the stated percentage or ratio. In some aspects, percentages and ratios associated with glycosidic linkages of oligosaccharide compositions include variations of ±1% of the stated percentage or ratio.

“Diabetes” is a disease that is characterized by hyperglycemia and relative lack of insulin. Diabetes is diagnosed when fasting plasma glucose ≥7.0 mmol/L (126 mg/dL), or two hours after dosing in a glucose tolerance test, plasma glucose ≥11.1 mmol/L (200 mg/dL). There are three main types namely Type 1 diabetes, Type 2 diabetes and gestational diabetes. Type 1 diabetes is an autoimmune disease that originates when beta cells that make insulin are destroyed by the immune system. Type 2 diabetes is caused by a combination of lifestyle and genetic factors leading to hyperglycemia, insulin resistance and finally impairment of the beta cells. Gestational diabetes occurs when a woman without diabetes develops high blood sugar levels during pregnancy. In this specification, unless a specific form is stated, the term diabetes encompasses all three forms of the disease.

“Obese” means a human individual that has a body mass index (BMI), a measurement obtained by dividing the individual's weight by the square of the individual's height, of over 30 kg/m².

“Overweight” means a human individual that has a body mass index (BMI), a measurement obtained by dividing the individual's weight by the square of the individual's height, in the range 25-30 kg/m².

The term “patient” or “subject” as used herein generally refers to a living organism suffering from or prone to a disease or condition that can be treated by administration of a compound or pharmaceutical composition, as provided herein. Non-limiting examples include humans, other mammals, bovines, rats, mice, dogs, monkeys, goat, sheep, cows, deer, and other non-mammalian animals. In some embodiments, a patient is human. In those aspects where the patient is a human, the patient can be a pediatric or adult patient. In some embodiments, a patient is a mammal. In some embodiments, a patient is a mouse. In some embodiments, a patient is an experimental animal. In some embodiments, a patient is a rat. In some embodiments, a patient is a test animal.

“Pre-diabetes” means that the individual has at least one of the following characteristics: a glycated hemoglobin (A1C) level between 5.7 and 6.4 percent, a fasting blood glucose from 100 to 125 mg/dL (5.6 to 7.0 mmol/L), or a blood sugar level from 140 to 199 mg/dl (7.8 to 11.0 mmol/L).

“Risk of diabetes” means having a risk factor indicative of a higher risk of developing diabetes. Non-limiting examples of risk factors include: a waistline of more than 100 cm for men or 88 cm for women, a blood pressure of 130/85 mmHg or higher, blood triglycerides levels above 150 mg/dl, fasting blood glucose levels greater than 100 mg/dl and high-density lipoprotein levels of less than 40 mg/dl in men or 50 mg/dl in women. An “individual at risk of diabetes” may have one or more of these factors present.

“Synthetic composition” means a composition which is artificially prepared and optionally means a composition containing at least one compound that is produced ex vivo chemically and/or biologically, e.g. by means of chemical reaction, enzymatic reaction or recombinantly. The synthetic composition typically comprises a beta-glucan oligosaccharide. Also, in some embodiments, the synthetic compositions may comprise one or more nutritionally or pharmaceutically active components which do not affect adversely the efficacy of the beta-glucan oligosaccharide. Some non-limiting embodiments of a synthetic composition are described below.

### DETAILED DESCRIPTION

In the following description, numerous specific details of the oligosaccharides, oligosaccharide compositions, and methods of the present invention are set forth in order to provide a thorough explanation of the precise nature of the invention. It will be apparent, however, to those of skill in the art that the invention can be practiced without these specific details. Although the following description is divided into sections, it is contemplated that each section contains various aspects of the invention and as such disclosure from within each section and across two or more sections can be combined to form any aspect of the invention.

In this description, it will be shown that by depolymerizing polysaccharide beta-glucan fibers, the oligosaccharide units may have an enhanced effect towards the inhibition of amylase, alpha-glucosidase, and SGLT1. Without wishing to be bound by any particular theory, it is believed that this increased effect may be caused by increased availability for the oligosaccharides to move into the active sites of the enzymes/transporters due to reduced substrate steric hindrance, more frequent interactions between the protein and the oligosaccharides, and lower solution viscosity. Further, the oligosaccharide units are more readily digested by the intestinal microbiota resulting in increased concentrations of short chain fatty acids in the colon. The beta-glucan oligosaccharides are also easier to formulate than the initial polysaccharides and have better organoleptics, improving subject compliance.

In some aspects, a method for treating glucose-related metabolic disorders in a subject is provided. In embodiments, glucose-related metabolic disorders include, diabetes (type 1, type 2 or gestational diabetes) or metabolic syndrome. In embodiments, a method for attenuating post-prandial glucose response in a subject is provided. In some embodiments, the subject is at risk of developing diabetes, is overweight and/or obese, is pregnant, and/or is diabetic. In some embodiments, a method for reducing the risk of a prediabetic and/or obese subject from progressing to type 2 diabetes is provided. In some embodiments, a method for lowering HbA1c levels in a subject is provided. In the forgoing embodiments, the method comprises enterally administering to the subject an effective amount of a beta-glucan oligosaccharide prior to and/or during the subject consuming a glucose source. In the method for lowering HbA1c levels in a subject, the post-prandial glucose response is attenuated in the subject over a period of at least 2 months by the enteral administration. In embodiments of the forgoing methods, the beta-glucan oligosaccharide is administered up to 2 hours prior to the subject consuming the glucose source. In embodiments of the forgoing methods, the beta-glucan oligosaccharide is administered up to 1 hour prior to the subject consuming the glucose source. In embodiments of the forgoing methods, the beta-glucan oligosaccharide is administered up to 30 minutes prior to the subject consuming the glucose source. In embodiments of the forgoing methods, the beta-glucan oligosaccharide is administered up to 15 minutes prior to the subject consuming the glucose source.

In embodiments, the effective amount of the beta-glucan oligosaccharide employed for treatment ranges from about 0.5 g to about 20 g. In embodiments, the effective amount of the beta-glucan oligosaccharide ranges from about 0.75 g to about 15 g. for example about 1 g to about 7.5 g.

The disclosure provide certain beta-glucan oligosaccharides and methods for their use. In embodiments, the beta-glucan oligosaccharide inhibits salivary or pancreatic amylase and/or the beta-glucan oligosaccharide inhibits the SGLT1 glucose transporter and/or the beta-glucan oligosaccharide inhibits alpha-glucosidase.

In embodiments, the beta-glucan oligosaccharide contains beta-1,3 and beta-1,4 linked glucose residues. In embodiments, the beta-glucan oligosaccharide contains both beta-1,3 and beta-1,4 linked glucose residues. In embodiments, the beta-glucan oligosaccharide comprises β1,3 linked glucose residues: $1.4 linked glucose residues in a ratio of 1:1 to 1:5, such as 1:1, 1:2, 1:3, 1:4, or 1:5. In embodiments, the beta-glucan oligosaccharide has a weight averaged molecular weight (Mw) of less than 10.000 Da, optionally greater than 500 Da or 1,000 Da. In embodiments, the beta-glucan oligosaccharide has a weight averaged molecular weight (Mw) of less than 8,000 Da, optionally greater than 500 Da or 1,000 Da. In embodiments, the beta-glucan oligosaccharide has a weight averaged molecular weight (Mw) of less than 7,500 Da, optionally greater than 500 Da or 1,000 Da. In embodiments, the beta-glucan oligosaccharide has a weight averaged molecular weight (Mw) of less than 5,000 Da, optionally greater than 500 Da or 1,000 Da. In embodiments, the beta-glucan oligosaccharide has a weight averaged molecular weight (Mw) of less than 2,500 Da, optionally greater than 500 Da or 1,000 Da. In embodiments, the beta-glucan oligosaccharide contains 3 to 30 subunits, wherein each subunit is either a beta-1,3 glucose residue or a beta-1,4 glucose residue. In embodiments, the beta-glucan oligosaccharide contains 3 to 30 subunits, wherein each subunit is either a beta-1,3 glucose residue or a beta-1,4 glucose residue. In embodiments, the beta-glucan oligosaccharide contains 3 to 30 subunits, wherein each subunit is a beta-1,3 glucose residue or a beta-1,4 glucose residue and the beta-glucan oligosaccharide contains both beta-1,3 glucose residues and beta-1,4 glucose residues. In embodiments, the beta-glucan oligosaccharide contains 3 to 30 subunits, or 3 to 25 subunits, or 5 to 30 subunits or 5 to 25 subunits, or 10 to 30 subunits or 10 to 25 subunits, or any subrange thereof.

In embodiments, the beta-glucan oligosaccharide has a dynamic viscosity ranging from about 1 to about 10 mPa*s at 100 mg/ml at 25° C. In embodiments, the beta-glucan oligosaccharide has a dynamic viscosity ranging from about 1 to about 5 mPa*s at 100 mg/ml at 25° C. In embodiments, the beta-glucan oligosaccharide has a dynamic viscosity ranging from about 1 to about 3 mPa*s at 100 mg/ml at 25° C. In embodiments, the beta-glucan oligosaccharide has a dynamic viscosity ranging from about 1 to about 1.5 mPa*s at 100 mg/ml at 25° C. In embodiments, the beta-glucan oligosaccharide has a dynamic viscosity of about 1.3 to about 1.4 mPa*s at 100 mg/ml at 25° C.

In embodiments, at least 70% of the mass of beta-glucan oligosaccharide has molecular mass of less than 100 kDa, optionally greater than 0.1 kDa, 0.5 kDa, or 1 kDa. In embodiments, at least 60% of the mass of the beta-glucan oligosaccharide has molecular mass of less than 50 kDa, optionally greater than 0.1 kDa, 0.5 kDa, or 1 kDa. In embodiments, at least 50% of the mass of the beta-glucan oligosaccharide has molecular mass of less than 15 kDa, optionally greater than 0.1 kDa. 0.5 kDa, or 1 kDa. In embodiments, at least 50% of the mass of the beta-glucan oligosaccharide has molecular mass of less than 5 kDa, optionally greater than 0.1 kDa, 0.5 kDa, or 1 kDa. In embodiments, at least 25% of the mass of the beta-glucan oligosaccharide has a molecular mass of less than 1 kDa, optionally greater than 0.1 kDa.

In embodiments, the beta-glucan oligosaccharide has a solubility of between 50 mg/ml and 1000 mg/ml, for example between 50 mg/ml and 1000 mg/ml, between 100 mg/ml and 1000 mg/ml, between 150 mg/ml and 1000 mg/ml, between 200 mg/ml and 1000 mg/ml, between 150 mg/ml and 500 mg/ml, or between 200 mg/ml and 1000 mg/ml. In preferred embodiments, the beta-glucan oligosaccharide has a solubility of at least 200 mg/ml, optionally less than 1000 mg/ml. In further preferred embodiments, the beta glucan oligosaccharide has a solubility of at least 200 mg/ml, optionally less than 1000 mg/ml, with a turbidity value of less than 20 NTU, optionally greater than 0.5 NTU.

In aspects, the compositions disclosed herein, including CLX compositions, have a turbidity value of between 0.5 NTU and 50 NTU, for example between 0.5 NTU and 50 NTU. between 0.5 NTU and 40 NTU, between 0.5 NTU and 30 NTU, between 0.5 NTU and 25 NTU, or between 0.5 NTU and 20 NTU. In preferred embodiments, the compositions disclosed herein, including CLX compositions, have a turbidity value of less than 20 NTU, optionally greater than 0.5 NTU, optionally greater than 1 NTU.

In embodiments, the disclosure provides methods for generating oligosaccharide compositions, particularly for generating beta-glucan oligosaccharides of this disclosure and for use as described herein. In embodiments, the beta-glucan oligosaccharide is generated by reacting polysaccharides in a reaction mixture with a Fenton's reagent, having a peroxide agent and metal ions, to provide treated polysaccharides; and cleaving the treated polysaccharides with a base to generate a mixture of polysaccharide cleavage products and/or oligosaccharides characteristic of the polysaccharides which mixture is the beta-glucan oligosaccharide. In embodiments, the Fenton's reagent comprises hydrogen peroxide, and one or more metal ions selected from the group consisting of transition metals Fe(II), Fe(III), Cu(I), Cu(II), Mn(II), Zn(II), Ni(II), and Co(II), alkaline earth metals Ca(II) and Mg(II), and the lanthanide Ce(IV). In embodiments, the base is one or more bases selected from the group consisting of ammonium hydroxide, ammonium bicarbonate, ammonia, urea, sodium amide, dimethyl amine, trimethylamine, pyridine, and N,N-diisopropylethylamine, sodium hydroxide, calcium hydroxide, potassium hydroxide, barium hydroxide, lithium hydroxide. In embodiments, the base is one or more bases selected from the group consisting of ammonium hydroxide and sodium hydroxide. In embodiments, the base is a nitrogen-based cleavage reagent. In embodiments, the nitrogen-based cleavage reagent is also a peroxide quenching reagent, and initiation of polysaccharide cleavage is commensurate, or substantially commensurate with initiation of peroxide-quenching. In embodiments, the nitrogen-based cleavage agent is not a peroxide-quenching agent, and the method further comprises initiation of peroxide quenching with an additional agent that is a peroxide-quenching agent. In embodiments, wherein the metal ion is a copper ion. In embodiments, the metal ion is Cu(II). In embodiments, Cu(II) is used in the reaction mixture at a concentration of about 0.25 mM to about 1.00 mM. In embodiments. Cu(II) is used in the reaction mixture at a concentration of about 0.7 mM to about 0.8 mM. In embodiments, Cu(II) is used in the reaction mixture at a concentration of about 0.75 mM. In embodiments, the source of Cu(II) is copper sulfate. In embodiments, the Fenton's reagent comprises copper sulfate. In embodiments, the hydrogen peroxide concentration is about 1% to about 7%. In embodiments, the hydrogen peroxide concentration is about 3.5 to 4.5% (v/v). In embodiments, the hydrogen peroxide concentration is about 4.0 (v/v). In embodiments, the base concentration is about 0.2 M to about 1M. In embodiments, the base concentration is about 0.3 M to about 0.5M. In embodiments, the base concentration is about 0.4 M. In embodiments, the base is added to a final pH of about 8 to 12. In embodiments, the base is added to a final pH of about 8.5 to 11. In embodiments, the base is added to a final pH of about 9.5 to 10.5. In embodiments, the base is added to a final pH of about 10. In embodiments, the polysaccharide loading in the reaction mixture is between about 2% and about 20% (w/v). In embodiments, the polysaccharide loading in the reaction mixture is between about 8% and about 12% (w/v). In embodiments, the polysaccharide loading is about 10% (w/v) in the reaction mixture.

In embodiments, the polysaccharide used to generate the beta-glucan oligosaccharide is derived from a grain. In embodiments, the polysaccharide used to generate the beta-glucan oligosaccharide is derived from oat or barley. In embodiments, the polysaccharide used to generate the beta-glucan oligosaccharide is a beta-glucan particularly a beta-glucan having a weight average molecular weight of 500 kDa or more, optionally less than 10,000 kDa. In some embodiments, the polysaccharide used to generate the beta-glucan oligosaccharide is derived from oat or barley co-product or waste streams, such as oat or barley milk solids, brewers spent grain, or pearled barley dust.

In embodiments, the beta-glucan is purified from the starting material via dry milling fractionation. In embodiments, the beta-glucan is purified by wet milling fractionating with centrifugation, decanters, tri-canters, and hydrocyclones. In embodiments, the beta-glucan is purified by removing starch via amylase digestion with or without jet-cooking or other steam cooking techniques. In embodiments, the beta-glucan is purified by removing proteins via protease digestion. Suitable proteases include flavourzyme, alcalase, papain, bromelain, protease A, protease B, neutrase, trypsin, chymotrypsin pepsin, and subtilisin. Enzyme liberated products such as glucose, maltose, maltooligosaccharides, peptides, and amino acids can be removed from the beta-glucan via ethanol precipitation or common solid liquid separation techniques (decanters, tri-canters, centrifuges, nutsche dryers) or size-based exclusion via membrane filtration, dead end-filtration, or chromatography techniques.

In another aspect, the disclosure provides for the use of a beta-glucan oligosaccharide as described herein for treating a glucose-related metabolic disorder or the use of a beta-glucan oligosaccharide as herein described for preparation of a medicament for treating a glucose-related metabolic disorder. In embodiments, the use is for treating diabetes or metabolic syndrome. In embodiments, the use is for treating type 1 or type 2 diabetes or gestational diabetes. In embodiments, the use is for attenuating post-prandial glucose response in a subject. In embodiments, the subject is at risk of developing diabetes, is overweight and/or obese, is pregnant, and/or is diabetic. In embodiments, the use is for reducing the risk of a prediabetic and/or obese subject from progressing to Type 2 diabetes. In embodiments, the use is for lowering HbA1c levels in a subject.

In an additional aspect, the disclosure provides, a pharmaceutical composition comprising a beta-glucan oligosaccharide as described herein and a pharmaceutically acceptable carrier.

In yet another aspect, the disclosure provides a dietary supplement comprising a beta-glucan oligosaccharide as described herein and a food-grade carrier.

In some aspects, a method for controlling glucose levels through the application of an oligosaccharide composition generated from cereal grain beta glucans is provided. In some aspects, the oligosaccharide composition is generated from oat, barley, rye, or wheat. In some aspects, the oligosaccharide composition is generated from cereal derived beta glucans. In some aspects, the oligosaccharide composition can disrupt the breakdown of starch. In some aspects, the oligosaccharide composition can disrupt the breakdown of maltooligosaccharides and maltose into glucose. In some aspects, the oligosaccharide composition can disrupt the absorption of glucose from the intestine. In some aspects, the oligosaccharide composition can inhibit salivary and pancreatic amylases. In some aspects, the oligosaccharide composition can inhibit alpha-glucosidase. In some aspects, the oligosaccharide composition can inhibit the SGLT1 transporter.

In some aspects, the oligosaccharide composition can be used to treat prediabetics. In some aspects, the oligosaccharide composition can be used to treat type 1 diabetes. In some aspects, the oligosaccharide composition can be used to treat type 2 diabetes. In some aspects, the oligosaccharide composition can be used to treat gestational diabetes. In some aspects, the oligosaccharide composition can be used to lower postprandial glucose spikes. In some aspects, the oligosaccharide composition can be used to lower HbA1c levels.

In some aspects, the oligosaccharide composition can be created by the depolymerization of beta glucans by Fenton systems. In some aspects, the Fenton systems use iron and/or copper. In some aspects, the composition can be created by enzymatic depolymerization. In some aspects, the enzymatic depolymerizations use lichenase, beta glucanase, and/or cellulase. In some aspects, the oligosaccharide composition is created through algae, yeast, and/or bacterial fermentation. In some aspects, the oligosaccharide composition is created by chemical or chemoenzymatic synthesis. In some aspects, the oligosaccharide composition is created by autolysis. In some aspects, the oligosaccharides are created by genetic engineering. In some aspects, the oligosaccharides are extracted from foodstuffs.

In some aspects, the oligosaccharide composition is fermented by the gut microbiome. In some aspects, the oligosaccharide composition stimulates the production of short chain fatty acids. In some aspects, the short chain fatty acids are butyrate or propionate or acetate or betahydroxybutyrate or lactate. In some aspects, the interaction between the oligosaccharide composition and the gut microbiome can help control blood glucose levels. In some aspects, the short chain fatty acids can stimulate the release of GLP-1. In some aspects, the GLP-1 stimulates insulin secretion. In some aspects, the oligosaccharide composition works synergistically by first inhibiting amylase, alpha-glucosidase, and SGLT1 in the small intestine before being fermented by the microbiome in the small intestine and colon.

In some aspects, the oligosaccharide contains β1-3 and β1-4 glucose glycosidic linkages. In some aspects, the oligosaccharide pool or at least one oligosaccharide within the pool contains a ratio of about 0.37:1 β1-3:81-4 glucose glycosidic linkages. In some aspects, the oligosaccharide pool or at least one oligosaccharide within the pool contains a ratio of about 0.20:1 to about 0.37:1 β1-3: β1-4 glucose glycosidic linkages. In some aspects, when measured by SEC-RID at least 70% of the mass is less than 100 kDa, optionally greater than 0.1 kDa or greater than 0.5 kDa. In some aspects, when measured by SEC-RID at least 60% of the mass is less than 50 kDa, optionally greater than 0.1 kDa or greater than 0.5 kDa. In some aspects, when measured by SEC-RID at least 50% of the mass is less than 15 kDa, optionally greater than 0.1 kDa or greater than 0.5 kDa. In some aspects, when measured by SEC-RID at least 50% of the mass is less than 5 kDa, optionally greater than 0.1 kDa or greater than 0.5 kDa. In some aspects, when measured by SEC-RID at least 25% of the mass is less than 1 kDa, optionally greater than 0.1 kDa or greater than 0.5 kDa. In some aspects, the oligosaccharide pool contains oligosaccharides ranging from a DP of 3-30.

In this specification, the following terms have the following meaning unless otherwise specified:

As used herein, the term “CLX101” refers to an oligosaccharide composition wherein 99% of the mass comprises glucose, as measured by hydrolytic monosaccharide compositional analysis. For composition CLX101, the glycosidic linkage composition comprises, approximately, the amount set forth in Table B, for CLX101 (75% 3-linked glucose, 9% terminal glucose, and 15% other minor linkages). The CLX101 composition comprises, approximately, the 1H-13C HSQC NMR correlations set forth in Table A for CLX101. The CLX101 composition comprises, approximately, the values set forth in in Table C, as measured by oligosaccharide analysis. CLX101 has a dynamic viscosity at 25° C. at 100 mg/ml of 1.306 mPa*s. In some aspects, CLX101 is derived from microbial curdlan. In some aspects, CLX101 generally is derived from microbial curdlan, but can also be derived from other materials/sources (e.g., depolymerization of polysaccharides or oligomerization of lower DP mono- and/or oligo-saccharides) that provide oligosaccharides that have the same (or substantially the same, e.g., values within 10%, or within 15%, or within 20%, or within 25%, or within 30%) dynamic viscosity, hydrolytic monosaccharide composition, glycosidic linkage composition, oligosaccharide analysis, and 1H-13C HSQC NMR analysis as CLX101. CLX101 was produced in accordance with the depolymerization described in Example 7.

As used herein, the term “CLX102” refers to an oligosaccharide composition wherein 37% of the mass comprises glucose, and 60% of the mass comprises mannose, as measured by hydrolytic monosaccharide compositional analysis. For composition CLX102, the glycosidic linkage composition comprises, approximately, the amount set forth in Table B, for CLX102 (32% 4-linked glucose, 8% terminal glucose, 48% 4-linked mannose, and 13% terminal mannose). The CLX102 composition comprises, approximately, the 1H-13C HSQC NMR correlations set forth in Table A for CLX102. The CLX102 composition comprises, approximately, the values set forth in Table D, as measured by oligosaccharide analysis. CLX102 has a dynamic viscosity at 25° C. at 100 mg/ml of 1.392 mPa*s. In some aspects, CLX102 generally is derived from Konjac glucomannan, but can also be derived from other materials/sources (e.g., depolymerization of polysaccharides or oligomerization of lower DP mono- and/or oligo-saccharides) that provide oligosaccharides that have the same (or substantially the same, e.g., values within 10%, or within 15%, or within 20%, or within 25%, or within 30%) dynamic viscosity, hydrolytic monosaccharide composition, glycosidic linkage composition, oligosaccharide analysis, and 1H-13C HSQC NMR analysis as CLX102. In an aspect, CLX102 was produced in accordance with the depolymerization described in Example 7.

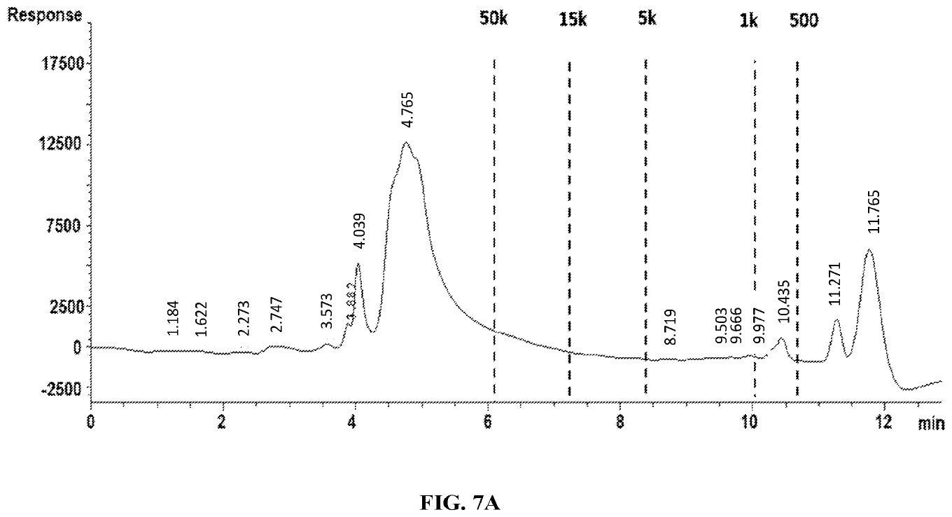

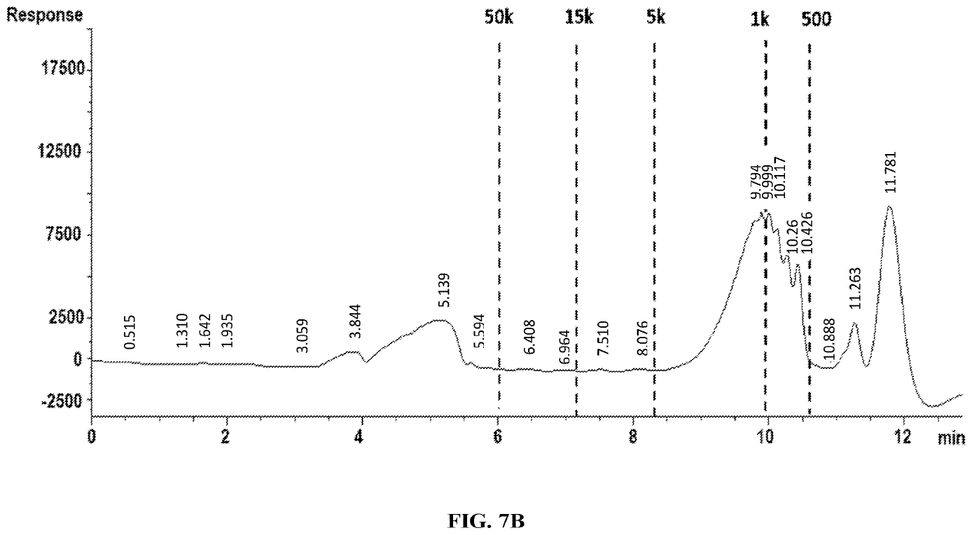

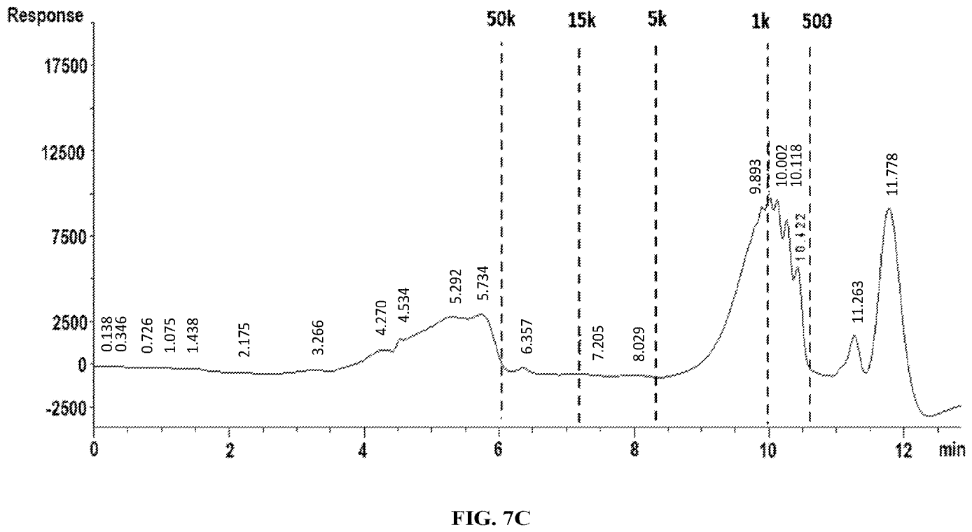

As used herein, the term “CLX112” refers to an oligosaccharide composition wherein 97% of the mass comprises glucose, as measured by hydrolytic monosaccharide compositional analysis. For composition CLX112, the glycosidic linkage composition comprises, approximately, the amount set forth in Table B, for CLX112 (17% 3-linked glucose, 49% 4-linked glucose, and 31% terminal glucose). The CLX112 composition comprises, approximately, the 1H-13C HSQC NMR correlations set forth in Table A for CLX112. The CLX112 composition comprises, approximately, the values set forth in Table E, as measured by oligosaccharide analysis. The molecular weight distribution of CLX112 composition comprises, approximately, the values set forth in Table K, as measured by refractive index detection (RID) (see also FIG. 7D). CLX112 has a dynamic viscosity at 25° C. at 100 mg/ml of 1.248 mPa*s. In some aspects, CLX112 generally is derived from barley beta glucan, but can also be derived from other materials/sources (e.g., depolymerization of polysaccharides or oligomerization of lower DP mono- and/or oligo-saccharides) that provide oligosaccharides that have the same (or substantially the same, e.g., values within 10%, or within 15%, or within 20%, or within 25%, or within 30%) dynamic viscosity, hydrolytic monosaccharide composition, glycosidic linkage composition, oligosaccharide analysis, and 1H-13C HSQC NMR analysis as CLX112. In an aspect, CLX112 was produced in accordance with the depolymerization described in Example 7.

As used herein, the term “CLX113” refers to an oligosaccharide composition wherein 49% of the mass comprises glucose, 36% of the mass comprises xylose, and 14% comprises galactose, as measured by hydrolytic monosaccharide compositional analysis. For composition CLX113, the glycosidic linkage composition comprises, approximately, the amount set forth in Table B, for CLX113 (28% 4-linked glucose, 6% 6-linked glucose, 20% 4,6-linked glucose, 4% terminal glucose, 21% terminal galactose, 6% 2-linked xylose, and 11% terminal xylose). The CLX113 composition comprises, approximately, the 1H-13C HSQC NMR correlations set forth in Table A for CLX113. The CLX113 composition comprises, approximately, the values set forth in Table F, as measured by oligosaccharide analysis. CLX113 has a dynamic viscosity at 25° C. at 100 mg/ml of 1.209 mPa*s. In some aspects, CLX113 generally is derived from tamarind seed xyloglucan. In some aspects, CLX113 is derived from other materials/sources (e.g., depolymerization of polysaccharides or oligomerization of lower DP mono- and/or oligo-saccharides) that provide oligosaccharides that have the same (or substantially the same, e.g., values within 10%, or within 15%, or within 20%, or within 25%, or within 30%) dynamic viscosity, hydrolytic monosaccharide composition, glycosidic linkage composition, oligosaccharide analysis, and 1H-13C HSQC NMR analysis as CLX113. In an aspect, CLX113 was produced in accordance with the depolymerization described in Example 7.

As used herein, the term “CLX115” refers to an oligosaccharide composition wherein 95% of the mass comprises glucose and 2% of the mass comprises arabinose, as measured by hydrolytic monosaccharide compositional analysis. For composition CLX115, the glycosidic linkage composition comprises, approximately, the amount set forth in Table B, for CLX115 (64% 4-linked glucose, 23% 3-linked glucose, and 13% terminal glucose). The CLX115 composition comprises, approximately, the 1H-13C HSQC NMR correlations set forth in Table A for CLX115. The CLX115 composition comprises, approximately, the values set forth in Table G, as measured by oligosaccharide analysis. The molecular weight distribution of CLX115 composition comprises, approximately, the values set forth in Table K, as measured by refractive index detection (RID) (see also FIG. 7C). CLX115 has a dynamic viscosity of 1.382 mPa*s at 100 mg/ml at 25° C. In some aspects, CLX115 generally is derived from oat beta glucan. In some aspects, CLX115 is derived from other materials/sources (e.g., depolymerization of polysaccharides or oligomerization of lower DP mono- and/or oligo-saccharides) that provide oligosaccharides that have the same (or substantially the same, e.g., values within 10%, or within 15%, or within 20%, or within 25%, or within 30%) dynamic viscosity, hydrolytic monosaccharide composition, glycosidic linkage composition, and oligosaccharide analysis as CLX115. In an aspect, CLX115 was produced in accordance with the depolymerization described in Example 7.

As used herein, the term “CLX123” refers to an oligosaccharide composition wherein 64% of the mass comprises glucose, 31% of the mass comprises mannose, and 3% of the mass comprises glucuronic acid, as measured by hydrolytic monosaccharide compositional analysis. For composition CLX123, the glycosidic linkage composition comprises, approximately, the amounts set forth in Table B for CLX123 (30% 4-linked glucose, 3% 3-linked glucose, 12% terminal glucose, 12% 3,6-linked galactose, 16% 2-linked mannose, 6% 4,6-linked mannose, 6% terminal mannose, and 4% 4-linked glucuronic acid). The CLX123 composition comprises, approximately, the 1H-13C HSQC NMR correlations set forth in Table A for CLX 123. The CLX123 oligosaccharide composition comprises, approximately, the values set forth in Table H, as measured by oligosaccharide analysis. CLX123 has a dynamic viscosity at 25° C. at 100 mg/ml of 1.658 mPa*s. In some aspects, CLX123 generally is derived from Xanthomonas Campestris extract. In some aspects, CLX123 is derived from any source or method (e.g., depolymerization of polysaccharides or oligomerization of lower DP mono- and/or oligo-saccharides) that provides oligosaccharides that have the same (or substantially the same, e.g., values within 10%, or within 15%, or within 20%, or within 25%, or within 30%) dynamic viscosity, hydrolytic monosaccharide composition, oligosaccharide analysis, glycosidic linkage composition, and 1H-13C HSQC NMR analysis as CLX 123. In an aspect, CLX123 was produced in accordance with the depolymerization described in Example 7.

As used herein, the term “CLX125” refers to an oligosaccharide composition wherein 44% of the mass comprises glucose, 43% of the mass comprises rhamnose, and 8% of the mass comprises glucuronic acid, as measured by hydrolytic monosaccharide compositional analysis. For composition CLX 125, the glycosidic linkage composition comprises, approximately, the amount set forth in Table B, for CLX125 (24% 4-linked glucose, 21% 3-linked glucose, 10% terminal glucose, 22% 4-linked rhamnose, 7% terminal rhamnose, 5% 4-linked glucuronic acid, and 3% terminal glucuronic acid). CLX125 composition comprises, approximately, the 1H-13C HSQC NMR correlations set forth in Table A for CLX125. The CLX125 oligosaccharide composition comprises, approximately, the values set forth in Table I, as measured by oligosaccharide analysis. CLX125 has a dynamic viscosity at 25° C. at 100 mg/ml of 1.525 mPa*s. In some aspects, CLX125 generally is derived from Sphingomonas elodea extract. In some aspects, CLX125 is derived from any source or method (e.g., depolymerization of polysaccharides or oligomerization of lower DP mono- and/or oligo-saccharides) that provides oligosaccharides that have the same (or substantially the same, e.g., values within 10%, or within 15%, or within 20%, or within 25%, or within 30%) dynamic viscosity, hydrolytic monosaccharide composition, oligosaccharide analysis, glycosidic linkage composition, and 1H-13C HSQC NMR analysis as CLX125. In an aspect, CLX125 was produced in accordance with the depolymerization described in Example 7.

As used herein, the term “CLX115-PS” (OatBG poly) refers to a polysaccharide composition wherein 99% of the mass comprises glucose as measured by hydrolytic monosaccharide compositional analysis. For composition CLX115-PS, the glycosidic linkage composition comprises, approximately, the amounts set forth in Table B for CLX 115-PS. The molecular weight distribution of CLX115-PS composition comprises, approximately, the values set forth in Table K, as measured by refractive index detection (RID) (see also FIG. 7A). In an aspect, oat beta-glucan polysaccharide for CLX115-PS was sourced from Purestar Chem in 90% purity. Oat beta-glucan has been reported to have a molecular weight of 65,000-3,100,000 Da (doi: 10.3390/ijms20164032).

As used herein, the term “CLX112-PS” (BarleyBG poly) refers to a polysaccharide composition wherein 97% of the mass comprises glucose as measured by hydrolytic monosaccharide compositional analysis. For composition CLX112-PS, the glycosidic linkage composition comprises, approximately, the amounts set forth in Table B for CLX112-PS. In an aspect, barley beta-glucan polysaccharide for CLX112-PS was sourced from Megazyme Inc. (product number P-BGBH). Barley beta-glucan has been reported to have a molecular weight of 31,000-2,700,000 Da (doi: 10.3390/ijms20164032).

As used herein, the term “CLX115Cu” refers to an oligosaccharide composition wherein 87.5% of the mass comprises glucose and 4.5% of the mass comprises arabinose, as measured by hydrolytic monosaccharide compositional analysis. For composition CLX115Cu, the glycosidic linkage composition comprises, approximately, the amount set forth in Table B, for CLX115Cu. The CLX115Cu oligosaccharide composition comprises, approximately, the values set forth in Table J, as measured by oligosaccharide analysis. The molecular weight distribution of CLX115Cu composition comprises, approximately, the values set forth in Table K, as measured by refractive index detection (RID) (see also FIG. 7B). In some aspects, CLX115 generally is derived from oat beta glucan. In some aspects, CLX115Cu is derived from other materials/sources (e.g., depolymerization of polysaccharides or oligomerization of lower DP mono- and/or oligo-saccharides) that provide oligosaccharides that have the same (or substantially the same, e.g., values within 10%, or within 15%, or within 20%, or within 25%, or within 30%) dynamic viscosity, hydrolytic monosaccharide composition, glycosidic linkage composition, and oligosaccharide analysis as CLX115Cu. In an aspect, CLX115Cu is produced in accordance with the depolymerization described in Example 6.

TABLE A
1H-13C HSQC NMR correlations from oligosaccharide compositions used herein.
The listed pairs correspond to those major peaks in the anomeric region.

| CLX101 | | CLX102 | | CLX113 | | CLX112 | | CLX115 | |
|---|---|---|---|---|---|---|---|---|---|
| 1H δ | 13C δ | 1H δ | 13C δ | 1H δ | 13C δ | 1H δ | 13C δ | 1H δ | 13C δ |
| [ppm] | [ppm] | [ppm] | [ppm] | [ppm] | [ppm] | [ppm] | [ppm] | [ppm] | [ppm] |
| 3.37 | 103.44 | 4.52 | 102.44 | 4.56 | 104.43 | 4.64 | 95.88 | 4.96 | 100.76 |
| 3.42 | 99.57 | 4.55 | 96.58 | 4.56 | 102.45 | 4.75 | 102.73 | 4.93 | 91.79 |
| 3.52 | 105.61 | 4.56 | 96.52 | 4.57 | 96.57 | 4.67 | 95.70 | 4.86 | 92.07 |
| 3.52 | 98.05 | 4.62 | 95.88 | 4.67 | 103.00 | 4.76 | 102.68 | 4.38 | 103.66 |
| 3.57 | 103.32 | 4.65 | 95.76 | 4.67 | 95.68 | 4.78 | 102.50 | 4.33 | 102.65 |
| 3.74 | 90.66 | 4.66 | 95.76 | 4.68 | 95.75 | 4.79 | 102.50 | 4.32 | 96.44 |
| 3.76 | 90.61 | 4.76 | 99.98 | 4.69 | 95.75 | 5.23 | 91.89 | 4.28 | 102.79 |
| 3.80 | 84.04 | 4.84 | 96.82 | 4.70 | 95.68 | 4.65 | 95.88 | 4.28 | 96.71 |
| 3.92 | 90.66 | 4.86 | 96.76 | 4.96 | 98.83 | 4.52 | 102.50 | 4.27 | 104.10 |
| 3.95 | 90.66 | 4.88 | 93.65 | 5.18 | 98.62 | 4.51 | 102.50 | 4.22 | 96.94 |
| 4.64 | 95.88 | 4.90 | 93.65 | 5.24 | 91.79 | 4.54 | 102.32 | 4.20 | 103.24 |
| 4.65 | 95.88 | 5.17 | 93.77 | 5.41 | 92.06 | 4.66 | 95.70 | | |
| 4.67 | 95.64 | 5.21 | 91.84 | | | | | | |
| 4.69 | 95.64 | 5.27 | 92.30 | | | | | | |
| 4.76 | 102.73 | 5.39 | 99.57 | | | | | | |
| 4.77 | 102.68 | | | | | | | | |
| 4.80 | 102.50 | | | | | | | | |
| 4.81 | 102.50 | | | | | | | | |
| 5.24 | 92.01 | | | | | | | | |

| | |
|---|---|
| CLX123 | CLX125 |

| 1H δ | 13C δ | 1H δ | 13C δ |
|---|---|---|---|
| [ppm] | [ppm] | [ppm] | [ppm] |
| 5.01 | 93.22 | 5.12 | 99.92 |
| 5.00 | 101.92 | 5.08 | 100.49 |
| 4.97 | 102.02 | 5.05 | 100.46 |
| 4.97 | 102.29 | 5.01 | 100.49 |
| 4.91 | 93.19 | 5.00 | 101.01 |
| 4.88 | 94.14 | 4.97 | 100.95 |
| 4.87 | 95.07 | 4.90 | 92.50 |
| 4.74 | 101.59 | 4.88 | 92.79 |
| 4.73 | 99.99 | 4.80 | 94.54 |
| 4.71 | 101.77 | 4.43 | 104.69 |
| 4.66 | 99.74 | 4.36 | 105.04 |
| 4.64 | 99.82 | 4.30 | 102.1 |
| 3.42 | 99.83 | 3.93 | 88.01 |
| 3.22 | 97.84 | 3.75 | 79.18 |
| | | 3.70 | 67.26 |
| | | 3.65 | 66.74 |
| | | 3.63 | 67.51 |
| | | 3.54 | 67.16 |
| | | 3.54 | 66.61 |
| | | 3.52 | 67.48 |

TABLE B
Glycosidic linkage analysis of CLX compositions. Data are presented in units of
peak area %. “Other” refers to linkages making up less than 2%. The notation
“—” represents a linkage that exists in an amount less than 2% (which
can be 0%) of the total oligosaccharide weight. If linkage is not fully described
it will be denoted by the monosaccharide, when known, or the type of monosaccharide,
either pentose or hexose, followed by multiple or a single “x” denoting
the number of branch points. Blank cells = not measured/no signal

| CLX No. | 3-Glc | 4-Glc | 6-Glc | 4,6-Glc | T-Glc | T-Man |
|---|---|---|---|---|---|---|
| CLX123 | 3.28 | 29.88 | — | — | 11.54 | 5.53 |
| CLX125 | 20.96 | 24.24 | — | — | 10.25 | — |
| CLX115 | 23 | 64 | | | 13 | |
| CLX115Cu | — | 64.41 | | | 16.42 | |
| CLX115-PS | 25.69 | 71.71 | | | 2.6 | |
| CLX112-PS | 27.77 | 71.88 | | | 0.35 | |
| CLX112 | 17 | 49 | | | 31 | |
| CLX101 | 75 | | | | 9 | |
| CLX102 | | 32 | | | 8 | |
| CLX113 | | 28 | 6 | 20 | 4 | |

| CLX No. | 4-Gal | 3,6-Gal | T-Gal | 2-Xyl | T-Xyl | 2-Man |
|---|---|---|---|---|---|---|
| CLX123 | | 12.24 | | | | 16.02 |
| CLX125 | | — | | | | — |
| CLX115 | | | | | | |
| CLX115Cu | 9.27 | | — | | — | |
| CLX115-PS | | | | | | |
| CLX112-PS | | | | | | |
| CLX112 | | | | | | |
| CLX101 | | | | | | |
| CLX102 | | | | | | |
| CLX113 | | | 21 | 6 | 11 | |

| CLX No. | 4,6-Man | | 4-Man | T-Man | 4-Rham | T-Rham | | 4-GlcA | T-GlcA | Other |
|---|---|---|---|---|---|---|---|---|---|---|
| CLX123 | 6.09 | | — | | — | — | — | 4.12 | — | 1.51 |
| CLX125 | — | | — | | — | 21.57 | 7.33 | 4.91 | 2.5 | 1.16 |
| CLX115 | | | | | | | | | | |
| CLX115Cu | | | | | | | | | | 5.96 |
| CLX115-PS | | | | | | | | | | |
| CLX112-PS | | | | | | | | | | |
| CLX112 | | | | | | | | | | |
| CLX101 | | | | | | | | | | 15 |
| CLX102 | | 48 | 13 | | | | | | | |
| CLX113 | | | | | | | | | | 4 |

TABLE C
CLX101 comprises the oligosaccharides shown in this table. Hex refers
to hexose sugars, Pent refers to pentose sugars, HexA refers to
hexuronic acid sugars, and Deoxyhex refers to deoxyhexose sugars.

| | | | RT | Oligo | Retention |
|---|---|---|---|---|---|
| Compound | Identity | Mass | (min) | Wt. % | Factor |
| 1 | 1Hex1Pent | 312.12 | 1.663 | 0.75 | 1.14 |
| 2 | 2Hex | 342.13 | 1.456 | 3.34 | 1.00 |
| 3 | 2Hex1Pent | 474.17 | 12.672 | 4.45 | 8.70 |
| 4 | 2Hex1Pent | 474.17 | 13.644 | 1.57 | 9.37 |
| 5 | 3Hex | 504.18 | 7.423 | 0.85 | 5.10 |
| 6 | 3Hex | 504.19 | 11.902 | 38.62 | 8.17 |
| 7 | 3Hex1Pent | 636.23 | 24.761 | 0.98 | 17.01 |
| 8 | 4Hex | 666.24 | 24.35 | 20.24 | 16.72 |
| 9 | 5Hex | 828.29 | 30.063 | 18.87 | 20.65 |
| 10 | 6Hex | 990.34 | 36.833 | 10.32 | 25.30 |

TABLE D
CLX102 comprises the oligosaccharides shown in this table. Hex refers
to hexose sugars, Pent refers to pentose sugars, HexA refers to
hexuronic acid sugars, and Deoxyhex refers to deoxyhexose sugars.

| | | | RT | Oligo | Retention |
|---|---|---|---|---|---|
| Compound | Identity | Mass | (min) | Wt. % | Factor |
| 1 | 2Hex1Pent | 474.17 | 2.263 | 0.17 | 1.518 |
| 2 | 2Hex1Pent | 474.17 | 9.187 | 0.88 | 6.162 |
| 3 | 2Hex1Pent | 474.17 | 10.653 | 0.54 | 7.145 |
| 4 | 2Hex1Pent | 474.17 | 12.713 | 0.37 | 8.526 |
| 5 | 3Hex | 504.18 | 1.491 | 0.84 | 1 |
| 6 | 3Hex | 504.18 | 2.193 | 0.35 | 1.471 |
| 7 | 3Hex | 504.19 | 6.695 | 4.31 | 4.49 |
| 8 | 3Hex | 504.19 | 7.637 | 2.06 | 5.122 |
| 9 | 3Hex | 504.18 | 8.885 | 1.94 | 5.959 |
| 10 | 3Hex | 504.19 | 9.605 | 1.74 | 6.442 |
| 11 | 3Hex | 504.19 | 12.868 | 1.45 | 8.63 |
| 12 | 3Hex | 504.18 | 14.159 | 0.4 | 9.496 |
| 13 | 3Hex1Pent | 636.23 | 6.413 | 0.92 | 4.301 |
| 14 | 3Hex1Pent | 636.23 | 12.074 | 1.13 | 8.098 |
| 15 | 3Hex1Pent | 636.23 | 13.068 | 0.77 | 8.765 |
| 16 | 3Hex1Pent | 636.23 | 18.947 | 1.52 | 12.708 |
| 17 | 3Hex1Pent | 636.23 | 19.573 | 0.75 | 13.127 |
| 18 | 3Hex1Pent | 636.23 | 20.258 | 0.81 | 13.587 |
| 19 | 4Hex | 666.24 | 4.124 | 2.33 | 2.766 |
| 20 | 4Hex | 666.24 | 6.206 | 2.04 | 4.162 |
| 21 | 4Hex | 666.24 | 9.373 | 2.99 | 6.286 |
| 22 | 4Hex | 666.24 | 10.25 | 2.66 | 6.875 |
| 23 | 4Hex | 666.24 | 10.97 | 3.94 | 7.357 |
| 24 | 4Hex | 666.24 | 14.977 | 1.75 | 10.045 |
| 25 | 4Hex | 666.24 | 16.802 | 4.87 | 11.269 |
| 26 | 4Hex | 666.24 | 17.38 | 5.13 | 11.657 |
| 27 | 4Hex | 666.24 | 19.079 | 1.69 | 12.796 |
| 28 | 4Hex | 666.24 | 19.911 | 0.58 | 13.354 |
| 29 | 4Hex | 666.24 | 20.691 | 0.36 | 13.877 |
| 30 | 4Hex | 666.24 | 24.147 | 1.15 | 16.195 |
| 31 | 4Hex1Pent | 798.28 | 9.604 | 0.45 | 6.441 |
| 32 | 4Hex1Pent | 798.28 | 15.28 | 0.48 | 10.248 |
| 33 | 4Hex1Pent | 798.28 | 17.219 | 0.45 | 11.549 |
| 34 | 4Hex1Pent | 798.28 | 18.793 | 0.64 | 12.604 |
| 35 | 4Hex1Pent | 798.28 | 20.328 | 1.41 | 13.634 |
| 36 | 4Hex1Pent | 798.28 | 21.012 | 0.36 | 14.093 |
| 37 | 4Hex1Pent | 798.28 | 21.923 | 0.44 | 14.704 |
| 38 | 4Hex1Pent | 798.28 | 22.714 | 0.55 | 15.234 |
| 39 | 4Hex1Pent | 798.28 | 26.723 | 0.35 | 17.923 |
| 40 | 4Hex1Pent | 798.28 | 27.138 | 0.22 | 18.201 |
| 41 | 4Hex1Pent | 798.28 | 27.347 | 0.26 | 18.341 |
| 42 | 4Hex1Pent | 798.28 | 27.565 | 0.32 | 18.488 |
| 43 | 5Hex | 828.29 | 7.648 | 1.05 | 5.129 |
| 44 | 5Hex | 828.29 | 9.213 | 1.28 | 6.179 |
| 45 | 5Hex | 828.29 | 12.21 | 0.37 | 8.189 |
| 46 | 5Hex | 828.29 | 14.335 | 1.28 | 9.614 |
| 47 | 5Hex | 828.29 | 14.932 | 0.75 | 10.015 |
| 48 | 5Hex | 828.29 | 16.24 | 2.01 | 10.892 |
| 49 | 5Hex | 828.29 | 17.527 | 1.3 | 11.755 |
| 50 | 5Hex | 828.29 | 18.549 | 5.98 | 12.441 |
| 51 | 5Hex | 828.29 | 18.882 | 0.36 | 12.664 |
| 52 | 5Hex | 828.29 | 19.204 | 1.29 | 12.88 |
| 53 | 5Hex | 828.29 | 19.522 | 0.69 | 13.093 |
| 54 | 5Hex | 828.29 | 20.062 | 1.8 | 13.455 |
| 55 | 5Hex | 828.29 | 20.313 | 1.44 | 13.624 |
| 56 | 5Hex | 828.29 | 21.885 | 1.43 | 14.678 |
| 57 | 5Hex | 828.29 | 25.896 | 3.46 | 17.368 |
| 58 | 5Hex | 828.29 | 27.822 | 0.56 | 18.66 |
| 59 | 5Hex | 828.29 | 28.03 | 0.64 | 18.799 |
| 60 | 6Hex | 990.34 | 11.067 | 0.31 | 7.423 |
| 61 | 6Hex | 990.34 | 11.999 | 0.4 | 8.048 |
| 62 | 6Hex | 990.34 | 15.579 | 0.48 | 10.449 |
| 63 | 6Hex | 990.34 | 17.343 | 0.5 | 11.632 |
| 64 | 6Hex | 990.34 | 18.939 | 0.55 | 12.702 |
| 65 | 6Hex | 990.34 | 19.881 | 1.01 | 13.334 |
| 66 | 6Hex | 990.34 | 21.075 | 0.83 | 14.135 |
| 67 | 6Hex | 990.34 | 21.86 | 0.73 | 14.661 |
| 68 | 6Hex | 990.34 | 22.178 | 0.72 | 14.875 |
| 69 | 6Hex | 990.34 | 22.854 | 2.01 | 15.328 |
| 70 | 6Hex | 990.34 | 23.366 | 1.15 | 15.671 |
| 71 | 6Hex | 990.34 | 24.012 | 1.16 | 16.105 |
| 72 | 6Hex | 990.34 | 25.898 | 0.92 | 17.37 |
| 73 | 6Hex | 990.34 | 26.344 | 0.71 | 17.669 |
| 74 | 6Hex | 990.34 | 28.735 | 0.53 | 19.272 |
| 75 | 6Hex | 990.34 | 28.984 | 0.59 | 19.439 |
| 76 | 7Hex | 1152.39 | 17.924 | 0.18 | 12.021 |
| 77 | 7Hex | 1152.4 | 22.096 | 0.58 | 14.82 |
| 78 | 7Hex | 1152.39 | 22.626 | 0.44 | 15.175 |
| 79 | 7Hex | 1152.4 | 22.915 | 0.27 | 15.369 |
| 80 | 7Hex | 1152.39 | 23.686 | 0.42 | 15.886 |
| 81 | 7Hex | 1152.39 | 24.151 | 0.37 | 16.198 |
| 82 | 7Hex | 1152.39 | 24.537 | 0.74 | 16.457 |
| 83 | 7Hex | 1152.39 | 25.769 | 0.59 | 17.283 |
| 84 | 7Hex | 1152.39 | 27.136 | 0.51 | 18.2 |
| 85 | 7Hex | 1152.39 | 28.252 | 0.34 | 18.948 |
| 86 | 7Hex | 1152.4 | 28.633 | 0.57 | 19.204 |
| 87 | 8Hex | 1314.45 | 24.955 | 0.37 | 16.737 |

TABLE E
CLX112 comprises the oligosaccharides shown in this table. Hex refers
to hexose sugars, Pent refers to pentose sugars, HexA refers to
hexuronic acid sugars, and Deoxyhex refers to deoxyhexose sugars.

| | | | RT | Oligo | Retention |
|---|---|---|---|---|---|
| Compound | Identity | Mass | (min) | Wt % | Factor |
| 1 | 3Hex | 504.19 | 10.431 | 7.9 | 1 |
| 2 | 3Hex | 504.19 | 14.158 | 13.53 | 1.357 |
| 3 | 3Hex | 504.19 | 17.074 | 5.47 | 1.637 |
| 4 | 4Hex | 666.24 | 21.248 | 11.27 | 2.037 |
| 5 | 4Hex | 666.24 | 24.78 | 6.29 | 2.376 |
| 6 | 4Hex | 666.24 | 25.028 | 8.82 | 2.399 |
| 7 | 4Hex | 666.24 | 25.857 | 9.81 | 2.479 |
| 8 | 5Hex | 828.29 | 27.544 | 2.08 | 2.641 |
| 9 | 5Hex | 828.29 | 28.172 | 3.8 | 2.701 |
| 10 | 5Hex | 828.29 | 28.914 | 1.18 | 2.772 |
| 11 | 5Hex | 828.29 | 29.507 | 7.33 | 2.829 |
| 12 | 5Hex | 828.29 | 30.241 | 11.24 | 2.899 |
| 13 | 6Hex | 990.34 | 32.369 | 2.62 | 3.103 |
| 14 | 6Hex | 990.34 | 34.032 | 6.74 | 3.263 |
| 15 | 6Hex | 990.34 | 37.05 | 1.95 | 3.552 |

TABLE F
CLX113 comprises the oligosaccharides shown in this table. Hex refers
to hexose sugars, Pent refers to pentose sugars, HexA refers to
hexuronic acid sugars, and Deoxyhex refers to deoxyhexose sugars.

| | | | RT | Oligo | Retention |
|---|---|---|---|---|---|
| Compound | Identity | Mass | (min) | Wt % | Factor |
| 1 | 2Hex1Pent | 474.17 | 6.596 | 8.71 | 1 |
| 2 | 2Hex1Pent | 474.17 | 7.182 | 1.54 | 1.089 |
| 3 | 2Hex1Pent | 474.17 | 9.915 | 6.7 | 1.503 |
| 4 | 2Hex2Pent | 606.22 | 10.489 | 0.41 | 1.59 |
| 5 | 2Hex2Pent | 606.22 | 14.055 | 8.77 | 2.131 |
| 6 | 3Hex1Pent | 636.23 | 11.569 | 2.3 | 1.754 |
| 7 | 3Hex1Pent | 636.23 | 12.735 | 4.64 | 1.931 |
| 8 | 3Hex1Pent | 636.23 | 17.858 | 2.32 | 2.707 |
| 9 | 3Hex1Pent | 636.23 | 19.639 | 2.6 | 2.977 |
| 10 | 3Hex1Pent | 636.23 | 21.047 | 2.57 | 3.191 |
| 11 | 3Hex2Pent | 768.27 | 16.236 | 1.77 | 2.461 |
| 12 | 3Hex2Pent | 768.27 | 16.913 | 7.56 | 2.564 |
| 13 | 3Hex2Pent | 768.27 | 22.6 | 4.71 | 3.426 |
| 14 | 3Hex2Pent | 768.27 | 23.712 | 4.79 | 3.595 |
| 15 | 3Hex2Pent | 768.27 | 24.427 | 2.85 | 3.703 |
| 16 | 3Hex3Pent | 900.31 | 25.675 | 1.94 | 3.893 |
| 17 | 4Hex1Pent | 798.28 | 19.976 | 2.44 | 3.029 |
| 18 | 4Hex1Pent | 798.28 | 22.29 | 1.57 | 3.379 |
| 19 | 4Hex1Pent | 798.28 | 26.972 | 0.62 | 4.089 |
| 20 | 4Hex1Pent | 798.28 | 27.808 | 0.91 | 4.216 |
| 21 | 4Hex1Pent | 798.28 | 28.061 | 0.79 | 4.254 |
| 22 | 4Hex2Pent | 930.32 | 18.582 | 2.65 | 2.817 |
| 23 | 4Hex2Pent | 930.32 | 22.74 | 1.7 | 3.448 |
| 24 | 4Hex2Pent | 930.32 | 23.905 | 1.69 | 3.624 |
| 25 | 4Hex2Pent | 930.32 | 24.564 | 3.31 | 3.724 |
| 26 | 4Hex2Pent | 930.32 | 24.966 | 3.86 | 3.785 |
| 27 | 4Hex2Pent | 930.32 | 27.736 | 1.18 | 4.205 |
| 28 | 4Hex2Pent | 930.32 | 28.44 | 0.98 | 4.312 |
| 29 | 4Hex2Pent | 930.32 | 28.778 | 1.97 | 4.363 |
| 30 | 4Hex2Pent | 930.32 | 29.18 | 1.24 | 4.424 |
| 31 | 4Hex2Pent | 930.32 | 30.836 | 0.75 | 4.675 |
| 32 | 4Hex3Pent | 1062.36 | 26.017 | 1.49 | 3.944 |
| 33 | 4Hex3Pent | 1062.36 | 26.225 | 1.2 | 3.976 |
| 34 | 4Hex3Pent | 1062.36 | 29.365 | 0.99 | 4.452 |
| 35 | 4Hex3Pent | 1062.36 | 29.76 | 0.96 | 4.512 |
| 36 | 4Hex3Pent | 1062.36 | 31.172 | 0.58 | 4.726 |
| 37 | 4Hex3Pent | 1062.36 | 31.49 | 1.02 | 4.774 |
| 38 | 5Hex3Pent | 1224.42 | 26.484 | 1.27 | 4.015 |
| 39 | 5Hex3Pent | 1224.42 | 28.91 | 0.54 | 4.383 |
| 40 | 5Hex3Pent | 1224.42 | 29.282 | 0.74 | 4.439 |
| 41 | 5Hex3Pent | 1224.42 | 30.009 | 0.89 | 4.55 |
| 42 | 5Hex3Pent | 1224.42 | 31.349 | 0.47 | 4.753 |

TABLE G
CLX115 comprises the oligosaccharides shown in this table. Hex refers
to hexose sugars, Pent refers to pentose sugars, HexA refers to
hexuronic acid sugars, and Deoxyhex refers to deoxyhexose sugars.

| Com- | | | RT | Oligos | Retention |
|---|---|---|---|---|---|
| pound | Identity | Mass | (min) | Wt % | Factor |
| 1 | 1Hex1Pent | 314.1214 | 1.839 | 0.09 | 1.196 |
| 2 | 1Hex2Pent | 446.1638 | 14.618 | 0.46 | 9.511 |
| 3 | 1Hex2Pent | 446.1641 | 14.73 | 0.17 | 9.584 |
| 4 | 1Hex2Pent | 446.1635 | 15.648 | 0.28 | 10.181 |
| 5 | 1Hex2Pent | 446.1643 | 17.222 | 0.11 | 11.205 |
| 6 | 2Hex | 344.1321 | 1.537 | 1.05 | 1 |
| 7 | 2Hex | 344.1329 | 2.878 | 2.41 | 1.872 |
| 8 | 2Hex1Pent | 476.1744 | 12.931 | 1.06 | 8.413 |
| 9 | 2Hex1Pent | 476.174 | 13.229 | 0.49 | 8.607 |
| 10 | 2Hex1Pent | 476.1753 | 13.732 | 1.53 | 8.934 |
| 11 | 2Hex1Pent | 476.1742 | 15.112 | 0.33 | 9.832 |
| 12 | 2Hex1Pent | 476.1748 | 16.113 | 0.84 | 10.483 |
| 13 | 2Hex1Pent | 476.1742 | 16.527 | 0.33 | 10.753 |
| 14 | 2Hex2Pent | 608.2157 | 24.944 | 0.45 | 16.229 |
| 15 | 2Hex2Pent | 608.2161 | 26.779 | 0.19 | 17.423 |
| 16 | 3Hex | 506.1856 | 3.295 | 0.63 | 2.144 |
| 17 | 3Hex | 506.1856 | 4.549 | 0.8 | 2.96 |
| 18 | 3Hex | 506.1843 | 10.5 | 0.08 | 6.831 |
| 19 | 3Hex | 506.1855 | 11.335 | 7.55 | 7.375 |
| 20 | 3Hex | 506.1855 | 14.01 | 0.23 | 9.115 |
| 21 | 3Hex | 506.1857 | 15.193 | 9.74 | 9.885 |
| 22 | 3Hex | 506.1857 | 15.445 | 0.05 | 10.049 |
| 23 | 3Hex | 506.184 | 15.874 | 0.08 | 10.328 |
| 24 | 3Hex | 506.1856 | 17.831 | 6.41 | 11.601 |
| 25 | 3Hex1HexA | 682.2171 | 25.124 | 0.13 | 16.346 |
| 26 | 3Hex1HexA | 682.2176 | 27.182 | 0.58 | 17.685 |
| 27 | 3Hex1HexA | 682.2177 | 27.272 | 0.19 | 17.744 |
| 28 | 3Hex1HexA | 682.2169 | 28.058 | 0.67 | 18.255 |
| 29 | 3Hex1Pent | 638.2261 | 9.335 | 0.1 | 6.074 |
| 30 | 3Hex1Pent | 638.2263 | 10.131 | 0.07 | 6.591 |
| 31 | 3Hex1Pent | 638.2276 | 23.161 | 0.93 | 15.069 |
| 32 | 3Hex1Pent | 638.2275 | 23.637 | 0.4 | 15.379 |
| 33 | 3Hex1Pent | 638.2273 | 24.298 | 0.44 | 15.809 |
| 34 | 3Hex1Pent | 638.2271 | 25.161 | 0.72 | 16.37 |
| 35 | 3Hex1Pent | 638.2282 | 25.768 | 0.84 | 16.765 |
| 36 | 3Hex1Pent | 638.2271 | 26.311 | 0.23 | 17.118 |
| 37 | 3Hex2Pent | 770.2696 | 29.593 | 0.06 | 19.254 |
| 38 | 3Hex2Pent | 770.2686 | 30.152 | 0.19 | 19.617 |
| 39 | 3Hex2Pent | 770.2681 | 30.838 | 0.07 | 20.064 |
| 40 | 3Hex2Pent | 770.2695 | 30.903 | 0.12 | 20.106 |
| 41 | 4Hex | 668.237 | 7.62 | 0.19 | 4.958 |
| 42 | 4Hex | 668.238 | 8.72 | 0.1 | 5.673 |
| 43 | 4Hex | 668.237 | 9.276 | 1.35 | 6.035 |
| 44 | 4Hex | 668.2351 | 9.819 | 0.06 | 6.388 |
| 45 | 4Hex | 668.2378 | 10.269 | 1.06 | 6.681 |
| 46 | 4Hex | 668.2379 | 10.63 | 0.34 | 6.916 |
| 47 | 4Hex | 668.2388 | 21.557 | 8.22 | 14.025 |
| 48 | 4Hex | 668.2386 | 24.211 | 0.1 | 15.752 |
| 49 | 4Hex | 668.239 | 25.244 | 4.57 | 16.424 |
| 50 | 4Hex | 668.239 | 25.507 | 5.92 | 16.595 |
| 51 | 4Hex | 668.2387 | 26.403 | 6.18 | 17.178 |
| 52 | 4Hex | 668.2343 | 26.607 | 0.04 | 17.311 |
| 53 | 4Hex1Deoxyhex | 814.2585 | 31.081 | 0.31 | 20.222 |
| 54 | 4Hex1HexA | 844.247 | 29.807 | 0.04 | 19.393 |
| 55 | 4Hex1Pent | 800.2787 | 28.827 | 0.23 | 18.755 |
| 56 | 4Hex1Pent | 800.2797 | 29.062 | 0.41 | 18.908 |
| 57 | 4Hex1Pent | 800.2787 | 29.552 | 0.11 | 19.227 |
| 58 | 4Hex1Pent | 800.2799 | 29.615 | 0.56 | 19.268 |
| 59 | 4Hex1Pent | 800.2788 | 30.227 | 1.2 | 19.666 |
| 60 | 5Hex | 830.2911 | 11.751 | 0.2 | 7.645 |
| 61 | 5Hex | 830.2895 | 13.261 | 0.48 | 8.628 |
| 62 | 5Hex | 830.2893 | 13.518 | 0.46 | 8.795 |
| 63 | 5Hex | 830.2899 | 13.84 | 0.71 | 9.005 |
| 64 | 5Hex | 830.2907 | 27.932 | 2.29 | 18.173 |
| 65 | 5Hex | 830.2922 | 28.423 | 3.57 | 18.493 |
| 66 | 5Hex | 830.2907 | 29.322 | 1.02 | 19.077 |
| 67 | 5Hex | 830.2908 | 29.82 | 4.98 | 19.401 |
| 68 | 5Hex | 830.291 | 30.469 | 6.13 | 19.824 |
| 69 | 6Hex | 992.3433 | 14.683 | 0.1 | 9.553 |
| 70 | 6Hex | 992.342 | 15.905 | 0.98 | 10.348 |
| 71 | 6Hex | 992.3433 | 31.896 | 1.52 | 20.752 |
| 72 | 6Hex | 992.3427 | 31.937 | 1.02 | 20.779 |
| 73 | 6Hex | 992.3434 | 33.337 | 0.52 | 21.69 |
| 74 | 6Hex | 992.3429 | 33.37 | 1.9 | 21.711 |
| 75 | 6Hex | 992.3429 | 33.763 | 0.91 | 21.967 |
| 76 | 6Hex | 992.3421 | 35.487 | 1.12 | 23.088 |

TABLE H
CLX123 comprises the oligosaccharides shown in this
table (Pent = pentose, generally arabinose,
and Hex = hexose, generally glucose or mannose).

| | | | RT | Oligo | Retention |
|---|---|---|---|---|---|
| Compound | Identity | Mass | (min) | Wt. % | Factor |
| 1 | 2Hex1HexA | 518.16 | 8.325 | 0.62 | 4.73 |
| 2 | 2Hex1HexA | 518.16 | 8.947 | 3.64 | 5.084 |
| 3 | 2Hex1Pent | 474.17 | 2.959 | 0.17 | 1.681 |
| 4 | 2Hex1Pent | 474.17 | 3.194 | 1.02 | 1.815 |
| 5 | 2Hex1Pent | 474.17 | 4.957 | 3.69 | 2.816 |
| 6 | 2Hex1Pent | 474.17 | 6.228 | 0.45 | 3.539 |
| 7 | 2Hex1Pent | 474.17 | 7.324 | 0.47 | 4.161 |
| 8 | 2Hex1Pent | 474.17 | 8.744 | 0.56 | 4.968 |
| 9 | 3Hex | 504.18 | 1.76 | 0.23 | 1 |
| 10 | 3Hex | 504.18 | 2.979 | 1.35 | 1.693 |
| 11 | 3Hex | 504.18 | 3.493 | 3.1 | 1.985 |
| 12 | 3Hex | 504.18 | 4.368 | 0.71 | 2.482 |
| 13 | 3Hex | 504.18 | 4.621 | 5.04 | 2.626 |
| 14 | 3Hex | 504.19 | 4.969 | 10.34 | 2.823 |
| 15 | 3Hex | 504.18 | 6.283 | 1.47 | 3.57 |
| 16 | 3Hex | 504.18 | 7.081 | 1.01 | 4.023 |
| 17 | 3Hex | 504.18 | 9.241 | 1.5 | 5.251 |
| 18 | 3Hex | 504.18 | 9.776 | 2.12 | 5.555 |
| 19 | 3Hex | 504.18 | 15.365 | 1.46 | 8.73 |
| 20 | 3Hex1HexA | 680.22 | 4.082 | 0.5 | 2.319 |
| 21 | 3Hex1HexA | 680.22 | 6.239 | 0.48 | 3.545 |
| 22 | 3Hex1HexA | 680.22 | 19.397 | 2.04 | 11.021 |
| 23 | 3Hex1Pent | 636.23 | 7.418 | 0.22 | 4.215 |
| 24 | 3Hex1Pent | 636.23 | 8.142 | 3.1 | 4.626 |
| 25 | 3Hex1Pent | 636.23 | 9.592 | 1.44 | 5.45 |
| 26 | 3Hex1Pent | 636.23 | 10.399 | 1.06 | 5.909 |
| 27 | 3Hex1Pent | 636.23 | 10.963 | 1.67 | 6.229 |
| 28 | 3Hex1Pent | 636.23 | 16.021 | 0.65 | 9.103 |
| 29 | 4Hex | 666.24 | 7.534 | 1.63 | 4.281 |
| 30 | 4Hex | 666.24 | 7.918 | 0.6 | 4.499 |
| 31 | 4Hex | 666.24 | 8.781 | 7.65 | 4.989 |
| 32 | 4Hex | 666.24 | 9.533 | 3.13 | 5.416 |
| 33 | 4Hex | 666.24 | 10.45 | 4.42 | 5.938 |
| 34 | 4Hex | 666.24 | 10.889 | 3.98 | 6.187 |
| 35 | 4Hex | 666.24 | 11.44 | 0.5 | 6.5 |
| 36 | 4Hex | 666.24 | 11.7 | 0.56 | 6.648 |
| 37 | 4Hex | 666.24 | 12.426 | 1.56 | 7.06 |
| 38 | 4Hex | 666.24 | 16.009 | 0.45 | 9.096 |
| 39 | 4Hex | 666.24 | 16.252 | 1.35 | 9.234 |
| 40 | 4Hex | 666.24 | 25.297 | 0.23 | 14.373 |
| 41 | 4Hex1HexA | 842.27 | 8.233 | 0.65 | 4.678 |
| 42 | 4Hex1HexA | 842.27 | 13.575 | 1.24 | 7.713 |
| 43 | 4Hex1Pent | 798.28 | 11.859 | 0.58 | 6.738 |
| 44 | 4Hex1Pent | 798.28 | 12.178 | 0.74 | 6.919 |
| 45 | 4Hex1Pent | 798.28 | 13.095 | 2.49 | 7.44 |
| 46 | 4Hex1Pent | 798.28 | 14.144 | 0.79 | 8.036 |
| 47 | 4Hex1Pent | 798.28 | 19.536 | 0.42 | 11.1 |
| 48 | 5Hex | 828.29 | 11.298 | 0.55 | 6.419 |
| 49 | 5Hex | 828.29 | 11.761 | 0.51 | 6.682 |
| 50 | 5Hex | 828.29 | 11.962 | 2.04 | 6.797 |
| 51 | 5Hex | 828.29 | 12.608 | 1.79 | 7.164 |
| 52 | 5Hex | 828.29 | 12.853 | 5.08 | 7.303 |
| 53 | 5Hex | 828.29 | 13.744 | 0.63 | 7.809 |
| 54 | 5Hex | 828.29 | 14.038 | 2.43 | 7.976 |
| 55 | 5Hex | 828.29 | 15.144 | 0.39 | 8.605 |
| 56 | 5Hex | 828.29 | 17.108 | 0.41 | 9.72 |
| 57 | 5Hex | 828.29 | 19.354 | 0.82 | 10.997 |
| 58 | 5Hex | 828.29 | 21.535 | 0.24 | 12.236 |
| 59 | 5Hex1Pent | 960.33 | 15.57 | 0.28 | 8.847 |
| 60 | 6Hex | 990.34 | 15.31 | 0.93 | 8.699 |
| 61 | 7Hex | 1152.39 | 16.851 | 0.77 | 9.574 |

TABLE I
CLX125 comprises the oligosaccharides shown in this table.

| | | | | | Reten- |
|---|---|---|---|---|---|
| Com- | | | RT | Oligo | tion |
| pound | Identity | Mass | (min) | Wt. % | Factor |
| 1 | 1Hex1HexA1Deoxyhex | 502.17 | 19.054 | 10.75 | 7.46 |
| 2 | 2Hex1Deoxyhex | 488.19 | 2.554 | 5.22 | 1 |
| 3 | 2Hex1HexA | 518.16 | 18.245 | 6.09 | 7.144 |
| 4 | 2Hex1HexA1Deoxyhex | 664.22 | 17.01 | 20.06 | 6.66 |
| 5 | 2Hex1HexA1Deoxyhex | 664.22 | 26.202 | 11.78 | 10.259 |
| 6 | 2Hex1HexA1Deoxyhex | 664.22 | 30.461 | 6.73 | 11.927 |
| 7 | 2Hex1HexA2Deoxyhex | 810.28 | 34.83 | 7.10 | 13.637 |
| 8 | 3Hex | 504.19 | 3.133 | 0.21 | 1.227 |
| 9 | 3Hex | 504.19 | 3.384 | 0.24 | 1.325 |
| 10 | 3Hex | 504.18 | 4.804 | 2.33 | 1.881 |
| 11 | 3Hex1HexA1Deoxyhex | 826.28 | 28.498 | 11.38 | 11.158 |
| 12 | 3Hex1HexA1Deoxyhex | 826.27 | 28.523 | 3.44 | 11.168 |
| 13 | 3Hex1HexA2Deoxyhex | 972.33 | 32.07 | 8.64 | 12.557 |
| 14 | 4Hex | 666.24 | 9.489 | 0.27 | 3.715 |
| 15 | 4Hex | 666.24 | 10.444 | 0.32 | 4.089 |
| 16 | 4Hex | 666.24 | 10.847 | 2.00 | 4.247 |
| 17 | 4Hex | 666.23 | 16.998 | 1.52 | 6.655 |
| 18 | 5Hex | 828.29 | 13.948 | 1.36 | 5.461 |
| 19 | 8Hex | 1314.46 | 26.197 | 0.55 | 10.257 |

TABLE J
CLX 115Cu comprises the oligosaccharides shown in this table.

| | | | RT | Oligo | Retention |
|---|---|---|---|---|---|
| Compound | Identity | Mass | (min) | Wt. % | Factor |
| 1 | 1Hex1Pent | 312.12 | 2.063 | 3.5 | 1.158 |
| 2 | 1Hex1Pent | 312.12 | 11.288 | 0.31 | 6.334 |
| 3 | 1Hex1Pent | 312.12 | 12.054 | 0.44 | 6.764 |
| 4 | 1Hex1Pent | 312.12 | 14.528 | 0.4 | 8.153 |
| 5 | 1Hex2Pent | 444.17 | 13.12 | 0.95 | 7.363 |
| 6 | 1Hex2Pent | 444.17 | 14.019 | 0.9 | 7.867 |
| 7 | 1Hex2Pent | 444.17 | 15.704 | 0.28 | 8.813 |
| 8 | 2Hex | 342.13 | 2.189 | 0.2 | 1.228 |
| 9 | 2Hex | 342.13 | 2.916 | 0.82 | 1.636 |
| 10 | 2Hex | 342.13 | 3.01 | 2.45 | 1.689 |
| 11 | 2Hex | 342.13 | 9.733 | 1.32 | 5.462 |
| 12 | 2Hex | 342.13 | 13.479 | 1.89 | 7.564 |
| 13 | 2Hex1Pent | 474.18 | 10.122 | 0.05 | 5.68 |
| 14 | 2Hex1Pent | 474.17 | 11.287 | 1.96 | 6.334 |
| 15 | 2Hex1Pent | 474.17 | 11.594 | 0.43 | 6.506 |
| 16 | 2Hex1Pent | 474.17 | 12.054 | 2.65 | 6.764 |
| 17 | 2Hex1Pent | 474.18 | 14.528 | 1.12 | 8.153 |
| 18 | 2Hex1Pent | 474.18 | 14.944 | 0.44 | 8.386 |
| 19 | 2Hex2Pent | 606.22 | 23.54 | 0.32 | 13.21 |
| 20 | 2Hex2Pent | 606.22 | 24.287 | 0.38 | 13.629 |
| 21 | 2Hex2Pent | 606.22 | 25.041 | 0.61 | 14.052 |
| 22 | 3Hex | 504.19 | 1.782 | 0.09 | 1 |
| 23 | 3Hex | 504.19 | 3.333 | 0.43 | 1.87 |
| 24 | 3Hex | 504.18 | 4.242 | 0.15 | 2.38 |
| 25 | 3Hex | 504.18 | 5.108 | 0.12 | 2.866 |
| 26 | 3Hex | 504.18 | 6.804 | 0.31 | 3.818 |
| 27 | 3Hex | 504.18 | 8.412 | 0.76 | 4.721 |
| 28 | 3Hex | 504.18 | 8.669 | 0.39 | 4.865 |
| 29 | 3Hex | 504.19 | 9.734 | 9.22 | 5.462 |
| 30 | 3Hex | 504.19 | 12.287 | 1.16 | 6.895 |
| 31 | 3Hex | 504.19 | 23.908 | 0.43 | 13.416 |
| 32 | 3Hex | 504.19 | 24.73 | 0.32 | 13.878 |
| 33 | 3Hex | 504.19 | 27.971 | 0.46 | 15.696 |
| 34 | 3Hex1Deoxyhex | 650.24 | 1.903 | 0.06 | 1.068 |
| 35 | 3Hex1HexA | 680.22 | 23.207 | 1.06 | 13.023 |
| 36 | 3Hex1HexA | 680.22 | 25.112 | 0.94 | 14.092 |
| 37 | 3Hex1HexA | 680.22 | 25.972 | 1.07 | 14.575 |
| 38 | 3Hex1Pent | 636.23 | 19.245 | 0.03 | 10.8 |
| 39 | 3Hex1Pent | 636.23 | 21.862 | 1.58 | 12.268 |
| 40 | 3Hex1Pent | 636.23 | 22.377 | 0.32 | 12.557 |
| 41 | 3Hex1Pent | 636.23 | 22.939 | 0.71 | 12.873 |
| 42 | 3Hex1Pent | 636.23 | 23.624 | 0.76 | 13.257 |
| 43 | 3Hex1Pent | 636.23 | 24.175 | 1.49 | 13.566 |
| 44 | 3Hex1Pent | 636.22 | 24.268 | 0.1 | 13.618 |
| 45 | 3Hex1Pent | 636.23 | 24.438 | 0.31 | 13.714 |
| 46 | 3Hex1Pent | 636.23 | 24.656 | 0.09 | 13.836 |
| 47 | 3Hex2Pent | 768.27 | 28.4 | 0.2 | 15.937 |
| 48 | 3Hex2Pent | 768.27 | 29.119 | 0.16 | 16.341 |
| 49 | 4Hex | 666.24 | 7.554 | 0.18 | 4.239 |
| 50 | 4Hex | 666.24 | 8.467 | 0.12 | 4.751 |
| 51 | 4Hex | 666.24 | 17.617 | 0.24 | 9.886 |
| 52 | 4Hex | 666.24 | 17.98 | 0.14 | 10.09 |
| 53 | 4Hex | 666.24 | 18.202 | 0.71 | 10.214 |
| 54 | 4Hex | 666.24 | 18.74 | 0.21 | 10.516 |
| 55 | 4Hex | 666.24 | 19.121 | 0.3 | 10.73 |
| 56 | 4Hex | 666.24 | 20.19 | 9.67 | 11.33 |
| 57 | 4Hex | 666.24 | 21.297 | 0.4 | 11.951 |
| 58 | 4Hex | 666.24 | 22.289 | 0.21 | 12.508 |
| 59 | 4Hex | 666.24 | 23.248 | 0.13 | 13.046 |
| 60 | 4Hex | 666.24 | 23.697 | 4.91 | 13.298 |
| 61 | 4Hex | 666.24 | 23.84 | 2.45 | 13.378 |
| 62 | 4Hex | 666.24 | 23.869 | 0.25 | 13.395 |
| 63 | 4Hex | 666.24 | 24.452 | 0.28 | 13.722 |
| 64 | 4Hex | 666.24 | 24.73 | 7.14 | 13.878 |
| 65 | 4Hex1HexA | 842.27 | 30.693 | 1.13 | 17.224 |
| 66 | 4Hex1Pent | 798.28 | 27.188 | 0.57 | 15.257 |
| 67 | 4Hex1Pent | 798.28 | 27.761 | 0.48 | 15.579 |
| 68 | 4Hex1Pent | 798.28 | 28.4 | 1.13 | 15.937 |
| 69 | 5Hex | 828.29 | 22.477 | 0.05 | 12.613 |
| 70 | 5Hex | 828.29 | 22.735 | 0.05 | 12.758 |
| 71 | 5Hex | 828.29 | 23.539 | 0.1 | 13.209 |
| 72 | 5Hex | 828.29 | 24.626 | 0.09 | 13.819 |
| 73 | 5Hex | 828.29 | 24.857 | 0.29 | 13.949 |
| 74 | 5Hex | 828.29 | 26.032 | 2.76 | 14.608 |
| 75 | 5Hex | 828.29 | 26.525 | 0.07 | 14.885 |
| 76 | 5Hex | 828.29 | 26.537 | 3.33 | 14.892 |
| 77 | 5Hex | 828.29 | 27.442 | 1.4 | 15.4 |
| 78 | 5Hex | 828.29 | 27.974 | 4.82 | 15.698 |
| 79 | 5Hex | 828.29 | 28.668 | 0.12 | 16.088 |
| 80 | 5Hex | 828.29 | 28.674 | 7.11 | 16.091 |
| 81 | 5Hex | 828.29 | 28.697 | 0.04 | 16.104 |
| 82 | 5Hex | 828.29 | 41.967 | 0.32 | 23.551 |
| 83 | 5Hex1Deoxyhex | 974.35 | 11.313 | 0.12 | 6.348 |
| 84 | 5Hex1Deoxyhex | 974.35 | 12.165 | 0.08 | 6.827 |
| 85 | 5Hex1Deoxyhex | 974.35 | 13.385 | 0.03 | 7.511 |
| 86 | 5Hex1Pent | 960.34 | 30.951 | 0.07 | 17.369 |
| 87 | 5Hex1Pent | 960.33 | 31.583 | 0.15 | 17.723 |
| 88 | 6Hex | 990.34 | 26.403 | 0.06 | 14.816 |
| 89 | 6Hex | 990.34 | 27.633 | 0.05 | 15.507 |
| 90 | 6Hex | 990.34 | 30.321 | 0.51 | 17.015 |
| 91 | 6Hex | 990.34 | 31.531 | 1.44 | 17.694 |
| 92 | 6Hex | 990.34 | 31.983 | 1.21 | 17.948 |
| 93 | 6Hex | 990.34 | 33.875 | 0.94 | 19.01 |

TABLE K
Refractive index detection (RID) analysis of CLX compositions.
Data are presented in units of peak area %.

| M.W. | | | | |
|---|---|---|---|---|
| Distribution | CLX 115-PS | CLX 115Cu | CLX 115 | CLX 112 |
| >100k | 65.88 | 21.19 | 21.12 | 11.65 |
| 100k-50k | 15.1 | 6.56 | 13.91 | 5.79 |
| 50k-15k | 7.5 | 0.74 | 3.18 | 1.68 |
| 15k-5k | 2.3 | 1.81 | 0.8 | 0.32 |
| 5k-1k | 2.73 | 33.71 | 23.21 | 28.34 |
| 1k-500 | 2.93 | 30.16 | 34.65 | 47.28 |
| <500 | 3.56 | 5.84 | 3.12 | 4.93 |

In an embodiment, this invention provides a method for attenuating post-prandial glucose response in a subject. The method comprises enterally administering to the subject an effective amount of a beta-glucan oligosaccharide prior to and/or during the subject consuming a glucose source. The beta-glucan oligosaccharide can be provided in the form of a synthetic composition. For example a formulation of substantially pure beta-glucan oligosaccharide, a supplement, a nutritional composition, or a drug.

In one embodiment, the synthetic composition is a dietary supplement in a unit dosage form containing a unit dose of the beta glucan oligosaccharide. The dietary supplement can contain an acceptable food-grade carrier, e.g. phosphate buffered saline solution, mixtures of ethanol in water, water and emulsions such as an oil/water or water/oil emulsion, as well as various wetting agents or excipients. The dietary supplement can also contain other materials that do not produce an adverse, allergic or otherwise unwanted reaction when administered to a human. The carriers and other materials can include solvents, dispersants, coatings, absorption promoting agents, controlled release agents, and one or more inert excipients, such as starches, granulating agents, microcrystalline cellulose, diluents, lubricants, binders, and disintegrating agents.

The dietary supplement can be administered orally, e.g. as a tablet, capsule, or pellet containing a predetermined amount of the beta-glucan oligosaccharide, or as a powder or granules containing a predetermined amount of the beta-glucan oligosaccharide or a gel, paste, solution, suspension, emulsion, syrup, bolus, electuary, or slurry, in an aqueous or non-aqueous liquid, containing a predetermined concentration of the beta-glucan oligosaccharide. An orally administered composition can include one or more binders, lubricants, inert diluents, flavoring agents, and humectants. An orally administered composition such as a tablet can optionally be coated and can be formulated to provide sustained, delayed or controlled release of the beta-glucan oligosaccharide.

The dietary supplement can also include active agents such as vitamins, minerals, prebiotics, probiotics, and anti-inflammatory agents. Examples of suitable vitamins include vitamins A, B-complex (such as B1, B2, B6 and B12), C, D, E and K, niacin and acid vitamins such as pantothenic acid, folic acid and biotin. Examples of suitable minerals include calcium, iron, zinc, magnesium, iodine, copper, phosphorus, manganese, potassium, chromium, molybdenum, selenium, nickel, tin, silicon, vanadium and boron. Examples of suitable prebiotics include human milk oligosaccharides, galacto-oligosaccharides, fructo-oligosaccharides, inulin, and the like. Suitable probiotics include B. animalis subsp. lactis BB-12, B. lactis HN019, B. lactis Bi07, B. infantis ATCC 15697, L. rhamnosus GG, L. rhamnosus HN001, L. acidophilus LA-5, L. acidophilus NCFM, L. fermentum CECT5716, B. longum BB536, B. longum AH 1205, B. longum AH1206, B. breve M-16V, L. reuteri ATCC 55730, L. reuteri ATCC PTA-6485, and L. reuteri DSM 17938. Suitable anti-inflammatory agents include natural antioxidants and polyphenols such as carotenoids, for example lutein, lycopene, zeaxanthin, and beta-carotene. Also, fats with anti-inflammatory properties such as omega-3 polyunsaturated fatty acids may be included.

The dietary supplement can also include one or more other agents suitable for use in foods and dietary supplements which improve glucose management. Suitable agents are well known in the art. Non-limiting examples include alpha-lipoic acid, chromium, magnesium, vanadium, cinnamon extract, berberine extract, fenugreek seed extract, Gymnema sylvestre extract, mulberry leaf extract, aloe vera extract, turmeric/curcumin extract, bilberry extract, bitter melon extract, ginseng extract, or any combination thereof.

The synthetic composition can also be formulated as unit dosage form for administration by naso-gastric tube or direct infusion into the GI tract or stomach.

In further embodiments, the synthetic composition can be formulated as a pharmaceutical composition. The pharmaceutical composition can contain a pharmaceutically acceptable carrier. e.g. phosphate buffered saline solution, mixtures of ethanol in water, water and emulsions such as an oil/water or water/oil emulsion, as well as various wetting agents or excipients. The pharmaceutical composition can also contain other materials that do not produce an adverse, allergic or otherwise unwanted reaction when administered to humans. The carriers and other materials can include solvents, dispersants, coatings, absorption promoting agents, controlled release agents, and one or more inert excipients, such as starches, granulating agents, microcrystalline cellulose, diluents, lubricants, binders, and disintegrating agents.

The pharmaceutical composition can also include one or more agents suitable for use in pharmaceuticals which improve glucose management. Suitable agents are well known in the art. Non-limiting examples include metformin, sulfonylureas (Glyburide, Glipizide, Glimepiride), Meglitinides (Repaglinide, Nateglinide), thiazolidinediones (Pioglitazone, Rosiglitazone), DPP-4 inhibitors (Sitagliptin, Saxagliptin, Linagliptin), SGLT-1 inhibitors (Canagliflozin, Dapagliflozin, Empagliflozin), GLP-1 receptor agonists (Semaglutide. Exenatide, Liraglutide, Dulaglutide), insulin, alpha-glucosidase blockers (Acarbose), or any combination thereof.

The pharmaceutical compositions can be administered orally. e.g. as a tablet, capsule, or pellet containing a predetermined amount, or as a powder or granules containing a predetermined concentration or a gel, paste, solution, suspension, emulsion, syrup, bolus, electuary, or slurry, in an aqueous or non-aqueous liquid, containing a predetermined concentration. Orally administered compositions can include binders, lubricants, inert diluents, flavoring agents, and humectants. Orally administered compositions such as tablets can optionally be coated and can be formulated to provide sustained, delayed or controlled release of the mixture therein.

The pharmaceutical compositions can also be administered by naso-gastric tube or direct infusion into the GI tract or stomach.

The pharmaceutical compositions can also include therapeutic agents such as antibiotics, probiotics, analgesics, and anti-inflammatory agents.

The synthetic composition can be in the form of a nutritional composition. For example, the nutritional composition can be a food composition, a rehydration solution, a medical food or food for special medical purposes, a nutritional supplement and the like. The nutritional composition can contain sources of protein, lipids and/or digestible carbohydrates and can be in powdered or liquid forms. The composition can be designed to be the sole source of nutrition or as a nutritional supplement.

Suitable protein sources include milk proteins, soy protein, rice protein, pea protein and oat protein, or mixtures thereof. Milk proteins can be in the form of milk protein concentrates, milk protein isolates, whey protein or casein, or mixtures of both. The protein can be whole protein or hydrolyzed protein, either partially hydrolyzed or extensively hydrolyzed. Hydrolyzed protein offers the advantage of easier digestion which can be important for humans with inflamed or compromised GI tracts. The protein can also be provided in the form of free amino acids. The protein can comprise about 5% to about 30% of the energy of the nutritional composition, normally about 10% to 20%. The protein source can be a source of glutamine, threonine, cysteine, serine, proline, or a combination of these amino acids.

Suitable digestible carbohydrates include maltodextrin, hydrolyzed or modified starch or corn starch, glucose polymers, corn syrup, corn syrup solids, high fructose corn syrup, rice-derived carbohydrates, pea-derived carbohydrates, potato-derived carbohydrates, tapioca, sucrose, glucose, fructose, sucrose, lactose, honey, sugar alcohols (e.g. maltitol, erythritol, sorbitol), or mixtures thereof. Preferably, the composition is reduced in or free from glucose or simple, digestible carbohydrates containing glucose. Generally digestible carbohydrates provide about 15% to about 55%, for example about 35% to about 55%, of the energy of the nutritional composition. A particularly suitable digestible carbohydrate is a low dextrose equivalent (DE) maltodextrin.

Suitable lipids include medium chain triglycerides (MCT) and long chain triglycerides (LCT). Generally, the lipids provide about 15% to about 50%, for example about 30% to about 50%, of the energy of the nutritional composition. The lipids can contain essential fatty acids (omega-3 and omega-6 fatty acids). Preferably, these polyunsaturated fatty acids provide less than about 30% of total energy of the lipid source.

Suitable sources of long chain triglycerides are rapeseed oil, sunflower seed oil, palm oil, soy oil, milk fat, corn oil, high oleic oils, and soy lecithin. Fractionated coconut oils are a suitable source of medium chain triglycerides. The lipid profile of the nutritional composition is preferably designed to have a polyunsaturated fatty acid omega-6 (n-6) to omega-3 (n-3) ratio of about 4:1 to about 10:1. For example, the n-6 to n-3 fatty acid ratio can be about 6:1 to about 9:1 (by weight).

The nutritional composition may also include vitamins and minerals. If the nutritional composition is intended to be a sole source of nutrition, it preferably includes a complete vitamin and mineral profile. Examples of vitamins include vitamins A, B-complex (such as B1, B2, B6 and B12), C, D, E and K, niacin and acid vitamins such as pantothenic acid, folic acid and biotin. Examples of minerals include calcium, iron, zinc, magnesium, iodine, copper, phosphorus, manganese, potassium, chromium, molybdenum, selenium, nickel, tin, silicon, vanadium and boron.

The nutritional composition can also include a carotenoid such as lutein, lycopene, zeaxanthin, and beta-carotene. The total amount of carotenoid included can vary from about 0.001 pg/ml to about 10 μg/ml. Lutein can be included in an amount of from about 0.001 μg/ml to about 10 μg/ml, preferably from about 0.044 μg/ml to about 5 μg/ml of lutein. Lycopene can be included in an amount from about 0.001 μg/ml to about 10 μg/ml, preferably about 0.0185 μg/ml to about 5 μg/ml of lycopene. Beta-carotene can comprise from about 0.001 μg/ml to about 10 mg/ml, for example about 0.034 μg/ml to about 5 μg/ml of beta-carotene.

The nutritional composition preferably also contains reduced concentrations of sodium; for example, from about 300 mg/l to about 400 mg/l. The remaining electrolytes can be present in concentrations set to meet needs without providing an undue renal solute burden on kidney function. For example, potassium is preferably present in a range of about 1180 to about 1300 mg/l; and chloride is preferably present in a range of about 680 to about 800 mg/l.

The nutritional composition can also contain various other conventional ingredients such as preservatives, emulsifying agents, thickening agents, buffers, fibers and prebiotics, probiotics, antioxidant/anti-inflammatory compounds including tocopherols, carotenoids, ascorbate/vitamin C, ascorbyl palmitate, polyphenols, glutathione, and superoxide dismutase (melon), other bioactive factors (e.g. growth hormones, cytokines, TFG-b), colorants, flavors, and stabilizers, lubricants, and so forth.

The nutritional composition can also include one or more other agents suitable for use in foods and dietary supplements which improve glucose management. Suitable agents are well known in the art. Non-limiting examples include alpha-lipoic acid, chromium, magnesium, vanadium, cinnamon extract, berberine extract, fenugreek seed extract, Gymnema sylvestre extract, mulberry leaf extract, aloe vera extract, turmeric/curcumin extract, bilberry extract, bitter melon extract, ginseng extract, or any combination thereof.

The nutritional composition can be formulated as a soluble powder, a liquid concentrate, or a ready-to-use formulation. The composition can be fed to a human in need via a nasogastric tube or orally. Various flavors, fibers and other additives can also be present.

The nutritional compositions can be prepared by any commonly used manufacturing techniques for preparing nutritional compositions in solid or liquid form. For example, the composition can be prepared by combining various feed solutions. A protein-in-fat feed solution can be prepared by heating and mixing the lipid source and then adding an emulsifier (e.g. lecithin), fat soluble vitamins, and at least a portion of the protein source while heating and stirring. A carbohydrate feed solution is then prepared by adding minerals, trace and ultra trace minerals, thickening or suspending agents to water while heating and stirring. The resulting solution is held for 10 minutes with continued heat and agitation before adding carbohydrates (e.g. the HMOs and digestible carbohydrate sources). The resulting feed solutions are then blended together while heating and agitating and the pH adjusted to 6.6-7.0, after which the composition is subjected to high-temperature short-time processing during which the composition is heat treated, emulsified and homogenized, and then allowed to cool. Water soluble vitamins and ascorbic acid are added, the pH is adjusted to the desired range if necessary, flavors are added, and water is added to achieve the desired total solid level.

For a liquid product, the resulting solution can then be aseptically packed to form an aseptically packaged nutritional composition. In this form, the nutritional composition can be in ready-to-feed or concentrated liquid form. Alternatively, the composition can be spray-dried and processed and packaged as a reconstitutable powder.

For attenuating post-prandial glucose response in a subject, the amount of beta-glucan oligosaccharide to be administered to the subject will vary depending upon factors such as risk factors, condition severity of the subject, the age of the subject, the form of the composition, the glucose load of and type of the food/beverage being consumed, and other medications being administered to the subject. However, the amount can be readily set by a medical practitioner. In certain embodiments, the dose to attenuate post-prandial glucose response may generally be in the range from about 0.5 g to about 20 g. for example from about 0.75 g to about 15 g, and more specifically from about 1 g to about 7.5 g. The beta-glucan oligosaccharide can be administered before the patient consumes a source of glucose or while consuming the source of glucose. When administered before consuming the source of glucose, the beta-glucan oligosaccharide can be administered up to about 30 minutes before, for example up to about 15 minutes before. When administered while consuming the source of glucose, the beta glucan oligosaccharide can be administered separately from the source of glucose or as a meal replacement. The daily dose of beta-glucan oligosaccharide administered to the subject would generally be about 0.5 g to about 20 g per day. For example, the daily dose can be about 0.75 g to about 15 g per day, more specifically about 1 g to about 10 g per day. An appropriate dose can be determined based on several factors, including, for example, body weight and/or condition, the severity of the condition, other ailments and/or diseases, the incidence and/or severity of side effects, the manner of administration, and/or the glucose load of the meal/beverage being consumed. Appropriate dose ranges may be determined by methods known to those skilled in the art.

For reducing the risk of a prediabetic and/or obese subject from progressing to Type 2 diabetes, the amount of beta-glucan oligosaccharide to be administered to the subject will vary depending upon factors, such as risk factors, condition severity of the subject, the age of the subject, the form of the composition, the glucose load of and type of the food/beverage being consumed, and other medications being administered to the subject. However, the amount can be readily set by a medical practitioner. Doses and daily doses as suggested above can be used. The beta-glucan oligosaccharide can be administered daily over a period of time, for example for at least one month, at least two months, etc.

For lowering HbA1c levels in a subject, the amount of beta-glucan oligosaccharide to be administered to the subject will vary depending upon factors such as risk factors, condition severity of the subject, the age of the subject, the form of the composition, the glucose load of and type of the food/beverage being consumed, and other medications being administered to the subject. However, the amount can be readily set by a medical practitioner. Doses and daily doses as suggested above can be used.

The synthetic composition can be co-administered to a patient who is also receiving a standard-of-care medication for glucose control.

### EXAMPLES

### Example 1: Evaluation of SGLT1 Receptor Inhibition by Beta-Glucan Oligosaccharides

It has been described that some polysaccharides reduce SGLT-1 expression not only in the intestinal mucosa of diabetic mice but also in Caco-2 cells. Furthermore, some long carbohydrate structures significantly promoted blood glucose regulation in normal mice, and reduced glucose transportation through Caco-2 monolayers (Cao et al. 2016). A method for measuring SGLT1 mediated transport is the cellular uptake assay. The interaction is detected as the modulation of the initial rate of 14C-AMG transport by human SGLT1 into HEK-293-FRT cells expressing the SGLT1 transporter. An inhibition test using phlorizin (100 μM) as the reference inhibitor was performed to ensure maximal inhibition of SGLT1 mediated AMG transport.

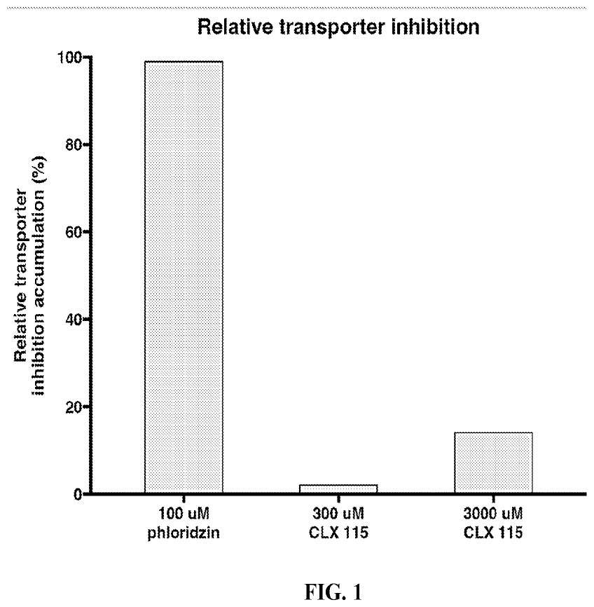

Beta-glucan oligosaccharides were produced from isolated oat beta-glucan from the method described in Example 8. Two different concentrations of beta-glucan oligosaccharides were tested in the assay (300 μM and 3000 μM). A dose dependent SGLT1 inhibition was observed when using beta-glucan oligosaccharides, with a value of 14% relative transporter inhibition at 3000 μM (FIG. 1). These results support that beta-glucan oligosaccharides may be an agent for reducing glucose uptake and thus for minimizing postprandial glucose levels.

### Example 2: Evaluation of Alpha-Glucosidases Inhibition

Alpha-Glucosidase is Localized in the brush border of the small intestine and is responsible for the enzymatic hydrolysis of starch, producing glucose as one of the main products. Alpha glucosidase is a target for the modulation of postprandial hyperglycemia, to help reduce post-meal blood glucose levels by arresting glucose absorption in the gut. Carbohydrate digestibility has been reported to relate to elevated postprandial blood glucose. One of the strategies to reduce postprandial hyperglycemia is to limit the activity of carbohydrate digestive enzymes in intestinal tract. «-Amylase is an enzyme that degrades the polymeric substrate into shorter oligomers by catalyzing the hydrolysis of α-1,4-glucan linkages present in starch, maltodextrins, and other related carbohydrates (Truscheit et al., 2010).

Inhibition of alpha-glucosidases by CLX115 was measured by using BioVision's alpha glucosidase inhibitor screening kit. It utilizes the ability of an active alpha-glucosidase to cleave a synthetic substrate thus, releasing a chromophore. In the presence of an alpha-glucosidase specific inhibitor, the enzymatic activity is reduced, detected by a decrease of absorbance readings. Nine carbohydrate sources were tested at various concentrations (Table L), using acarbose as the positive inhibition control. In a separate experiment testing only CLX115, reactions contained 100 mg/ml of CLX115 or 200 μM acarbose, 20 μM Maltose, and alpha glucosidase at 2 U/ml. Multiple tubes per treatment were prepared and incubated at 37° C. Progression of the reaction was captured by placing tubes in a 96° C. water bath for 10 min to inactivate the enzyme and stopping the reaction at 0, 10, 30 and 60 min. Glucose and Maltose were measured in the manner of Xu ct al. (10.1039/C7AN01530E). Briefly, glucose and maltose were derivatized with PMP (3-Methyl-1-phenyl-2-pyrazoline-5-one) and analyzed on an Agilent UHPLC/QqQ mass spectrometer. Quantitation is performed by employing a standard curve and peak area is normalized by employing internal standards.

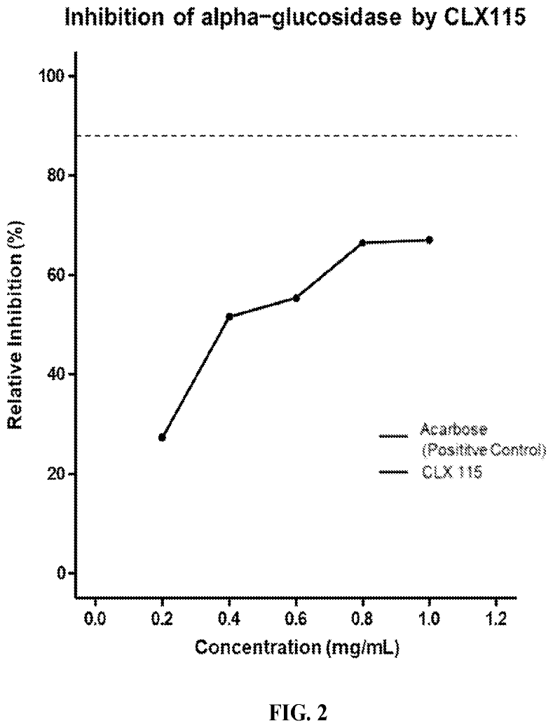

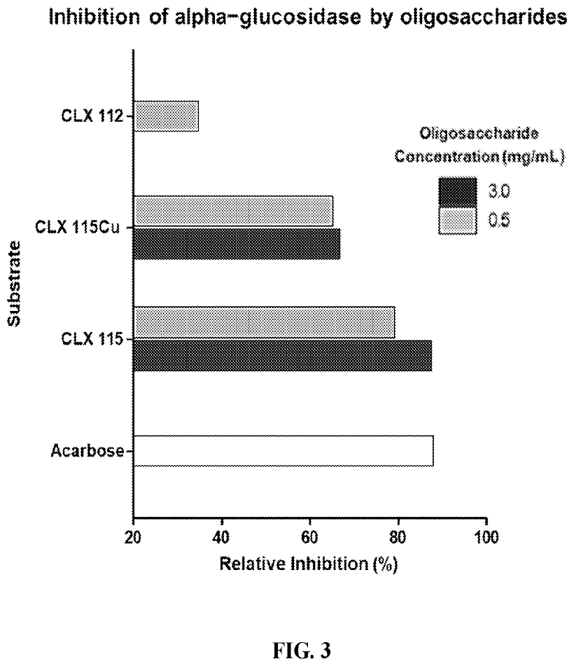

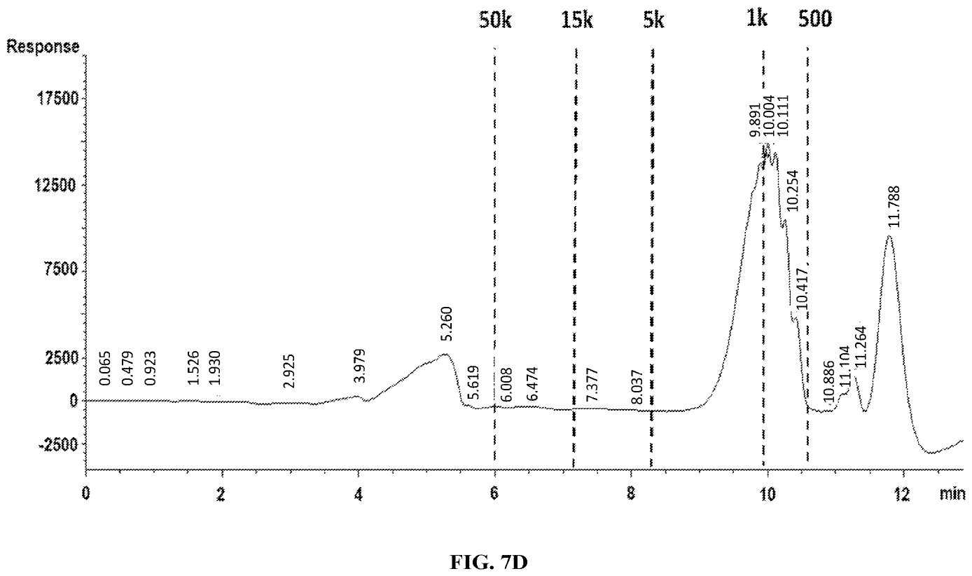

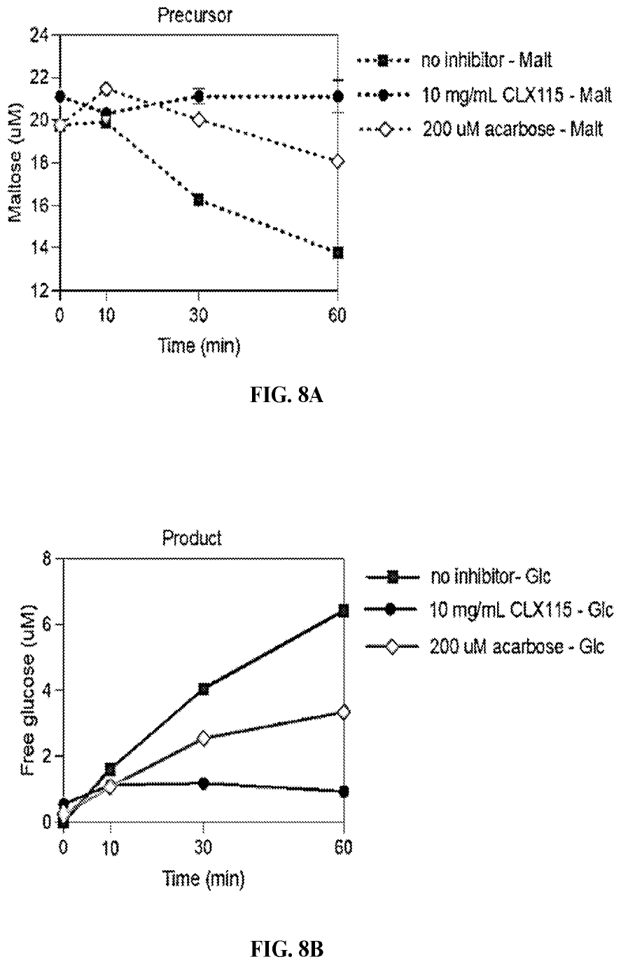

Results show that CLX115. CLX112, and CLX115Cu inhibit alpha-glucosidase at the tested concentrations, while various other carbohydrate sources, including the CLX115-PS did not show inhibition (Table L, FIG. 2, and FIG. 3). For the separate experiment testing only CLX115, when alpha-glucosidase was incubated in the presence of maltose alone, results show that maltose levels decrease (FIG. 8A) and free glucose is accumulated (FIG. 8B) as the reaction progressed. Both CLX115 and acarbose (positive control) suppressed the release of glucose (FIG. 8B) and inhibited the breakdown of maltose (FIG. 8A). In fact, in the presence of 100 mg/ml of CLX115, maltose and glucose levels were virtually constant until the end of the reaction. This demonstrates that CLX115 efficiently inhibits alpha-glucosidase activity. This inhibitory effect implies that CLX115 can act as an agent for lowering post-prandial glucose increases. CLX 115-PS was evaluated for alpha-glucosidase inhibition together with their oligosaccharide counterparts. However, the turbidity and viscosity of the polysaccharide solutions are important factors that could act as confounding variables. This inhibitory effect implies that depolymerized cereal beta-glucans, from various sources can act as an agent for reducing glucose uptake.

TABLE L
Alpha-glucosidase inhibition results.

| | 3.0 | 1.0 | 0.5 |
|---|---|---|---|
| CLX No. | mg/mL | mg/mL | mg/mL |
| CLX125 | NA | 3.79% | NA |
| CLX102 | 43.60% | 41.34% | 46.90% |
| CLX123 | 8.23% | X | NA |
| CLX113 | X | 5.84% | NA |
| CLX115 | 87.50% | 71.21% | 79.10% |
| CLX115Cu | 66.70% | NA | 65.10% |
| CLX115-PS | NA | X | X |
| CLX112 | NA | NA | 34.60% |
| CLX101 | 93.20% | NA | 96.20% |
| Acarbose | 87.90% | | |

NA = not measured.
X = not inhibitory

### Example 3: In Vitro Evaluation of Short Chain Fatty Acid Production by CLX115

Short chain fatty acids, particularly acetate, propionate, and butyrate, are mainly produced by anaerobic fermentation of gut microbes. SCFAs has demonstrated physiologically beneficial effects, like stimulating G-protein-coupled receptors. Studies have shown that the activation of GPR41 by SCFA can stimulate the secretion of intestinal hormone glucagon-like peptide-1 (GLP-1) which indirectly regulates blood glucose levels by increasing insulin secretion and decreasing pancreatic glucagon secretion.

One of the available gut models was used, the Simulator of the Human Intestinal Microbial Ecosystem (SHIME), to evaluate the effect of CLX115 in the production of SCFA. This system allowed simulation of physiology and microbiology of the GI tract. The typical reactor setup of the SHIME consists of a succession of three reactors; the first one stimulates the different steps in food uptake and digestion (representing stomach and small intestine). The other two reactors simulate the large intestine (proximal and distal colon) and are inoculated with fecal samples from a healthy donor. The experiment was divided in three different stages: control stage (2 weeks, stabilization of reactors and fecal sample, being the baseline microbial community and activity), treatment stage (3 weeks, CLX115 was added three times a day with the feed to simulate repeated intake, revealing the effects of the oligos) and wash out (2 weeks, to test permanence of the effect after interruption of the treatment). Samples were taking at different timepoints during the experiment and analyzed to determine levels of SCFA. SCFA (acetate, propionate and butyrate) and BCFA (isobutyrate, isovalerate and isocaproate) were determined by gas chromatography (GC 2014-AOC 20i autosampler, Shimadzu Europa GmbH) as recently published (Ghyselinck, Verstrepen et al. 2020).

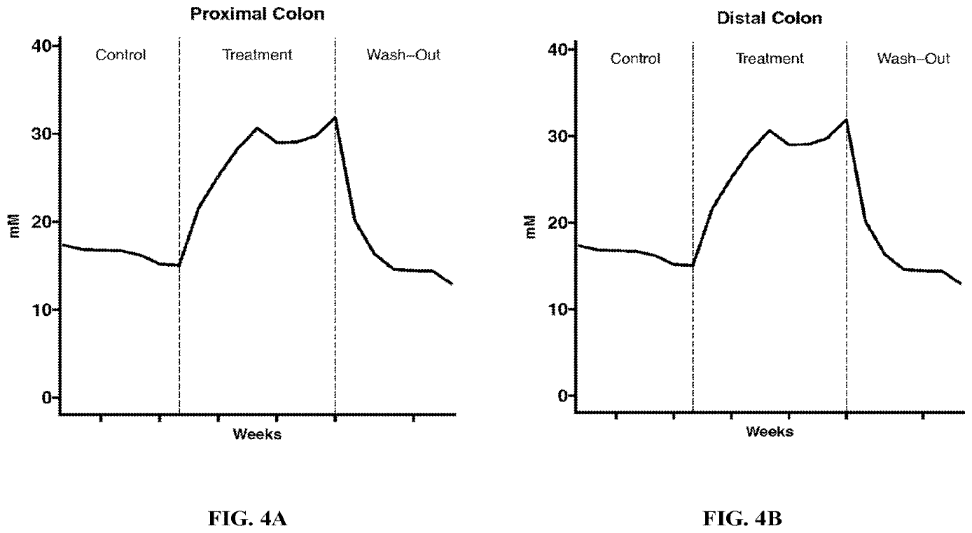

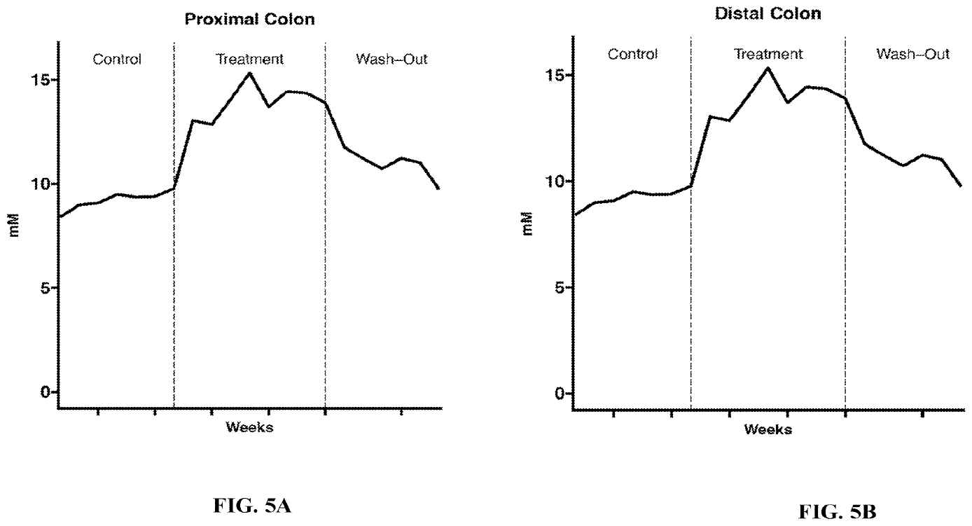

Results show a strong butyrogenic effect of CLX115 (FIGS. 4A (Proximal Colon) and 4B (Distal Colon)) as well as strong increase of propionic acid (FIGS. 5A (Proximal Colon) and 5B (Distal Colon)) during treatment period. No significant effect on acetate and BCFA was observed. After cessation of the treatment, concentrations of these SCFA generally returned to their pretreatment levels. The ability to increase SCFA production by CLX115 indicates that this oligosaccharide can act as an agent for stimulating GLP-1 production, which regulates glucose levels in blood.

Fermentative activity by gut microbes was determined by the measurement of gas production by the presence of CLX115. A short-term colonic simulation was performed in triplicate, using inoculum collected from the proximal colon vessel at the end of treatment phase during the SHIME experiment. We compared to treatment with FOS, and quantified total gas production. A negative control, not receiving any treatment will be included. The negative control was inoculated with sample from the control phase.

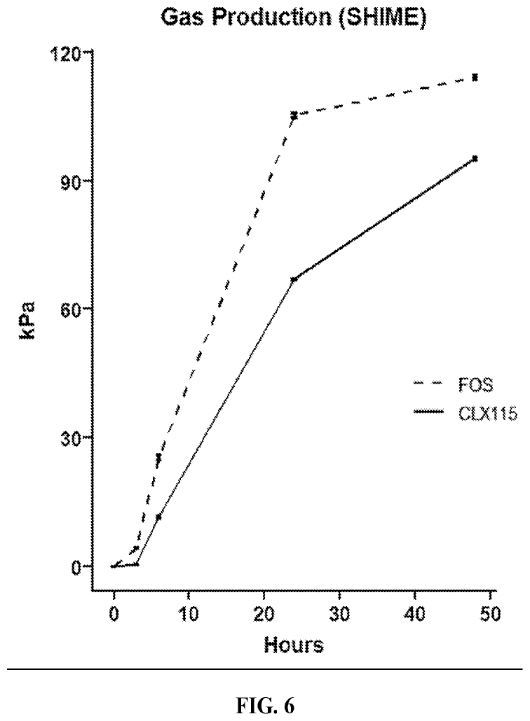

Gas formation was measured using a pressure meter to which the needle was connected (hand-held pressure indicator CPH6200; Wika, Echt, The Netherlands). Results show that CLX 115 produced less gas than FOS (Add %) (FIG. 6). This data indicates that CLX115, in this system, total gas production in vitro higher than 100 kPa has been related to bloating. Thus, CLX115 is an agent for glucose controls that provide less risk of bloating than common prebiotic FOS.

### Example 4: Optimization of Hydrogen Peroxide and Copper (II) Sulfate Concentration in Copper-Based Fenton Depolymerization of Beta-Glucan

The use of iron-based and copper-based Fenton depolymerization has been demonstrated in several previous publications (WO2021097138A1, WO2018236917A1, WO2020247389A1). However, a thorough optimization of copper-based Fenton depolymerization has never been demonstrated. Here, we show the impact of hydrogen peroxide concentration on the copper-based Fenton depolymerization of beta-glucan, particularly from cereals.

Beta Glucan was massed to 4 g in three different bottles followed by dissolution to a concentration of 10% in a 50 mM, pH 5.5 sodium acetate buffer containing either 2%, 3% or 4% hydrogen peroxide. The mix was heated to 55° C. in a shaker incubator. Copper (II) sulfate was added to start the reaction with a final concentration of either 0.25 mM, 0.50 mM, or 1.00 mM and allowed to run for 2 hours at 55° C. Following 2 hours the mixtures were removed from the incubator and chilled to below 15° C. Next, concentrated ammonium hydroxide was added to a final concentration of 0.39M and allowed to run in a shaker incubator at 45° C. for 2 hours. The reaction was halted through freezing at −80° C. and lyophilization to dryness. The lyophilized material was re-hydrated with minimal ultra-pure H₂O followed by dilution with 200 proof food-grade ethanol until a 60% ethanol solution was achieved. The suspension was extracted by centrifugation at 4700 rmp for 15 min at −10° C. followed by decanting the supernatant. The subsequent supernatant volume was reduced via rotary evaporation and lyophilization to yield a crystalline solid.

It was concluded that the reaction containing a concentration of 4% H₂O₂ produced the optimal oligosaccharide molecular weights at the highest yield. This increase in yield was accomplished while maintaining low free monosaccharide production and high purity. Additionally, the reaction required a concentration between 0.50 mM and 1.00 mM of Copper (II) Sulfate to provide the optimal oligosaccharide molecular weights at the highest yield. Therefore, it can be concluded that hydrogen peroxide and Copper (II) Sulfate concentration can be used to optimize for yield and/or molecular weight distribution of beta glucan oligosaccharides.

### Example 5: Optimization of Ammonium Hydroxide Concentration in Copper-Based Fenton Depolymerization of Beta-Glucan

The use of iron-based and copper-based Fenton depolymerization has been demonstrated in several previous publications (WO2021097138A1, WO2018236917A1, WO2020247389A1). However, a thorough optimization of copper-based Fenton depolymerization has never been demonstrated. Here, we show the impact of copper concentration on the copper-based Fenton depolymerization of beta-glucan, particularly from cereals.

A beaker containing a 50 mM, 5.50 pH ammonium acetate buffer was heated to 55° C. Next, Beta Glucan was stirred in slowly to a final concentration of 10% followed by the adding hydrogen peroxide to a concentration of 4%. Once the mixture reached 55° C., the reaction was started through addition of copper (II) sulfate to a concentration of 0.75 mM. The reaction was allowed to stir for 2 hours held at 55° C. then was promptly chilled to below 15° C. To begin the base cleavage step, concentrated ammonium hydroxide was added to a final concentration of either 0.58M or 0.76M. The reaction was run for 2 hours at 45° C. then stopped and put through vacuum filtration using a GD120 filter and Buchner funnel. The flow through was then treated with MB10 resin (10% w/v) to an electrical conductivity of ˜90 μS/cm was achieved. The resin was removed via vacuum filtration with a glass-fritted funnel followed by freezing and lyophilization of the filtrate to dryness. The lyophilized material was re-hydrated with minimal ultra-pure H₂O followed by dilution with 200 proof food-grade ethanol until a 60% ethanol solution was achieved. The suspension was extracted by centrifugation at 4700 rmp for 15 min at −10° C. followed by decanting the supernatant. The subsequent supernatant volume was reduced via rotary evaporation and lyophilization to yield a white, fluffy, crystalline solid.

It was concluded that the reaction containing a concentration of between 0.58M and 0.76M Ammonium hydroxide produced the near optimal oligosaccharide molecular weights at the highest yield. This increase in yield was accomplished while maintaining low free monosaccharide production and high purity. Therefore, it can be concluded that Ammonium hydroxide concentration can be used to optimize for yield and/or molecular weight distribution of beta glucan oligosaccharides.

### Example 6: Optimized Conditions for Copper-Based Fenton Depolymerization of Beta-Glucan

The use of iron-based and copper-based Fenton depolymerization has been demonstrated in several previous publications (WO2021097138A1, WO2018236917A1, WO2020247389A1). However, a thorough optimization of copper-based Fenton depolymerization has never been demonstrated. Here, we show a set of optimized parameters and provide instructions for the copper-based Fenton depolymerization of beta-glucan, particularly from cereals.

A solution containing 4% hydrogen peroxide with 43.4 mM, pH 5.5 ammonium acetate buffer is heated to 55° C. Beta Glucan (or other) polysaccharides are stirred in gradually to a final concentration of 10%. To initiate the first reaction step, Copper (II) sulfate is added to a final concentration of 0.75 mM. The reaction is allowed to proceed for 2 hours at 55° C. then cooled to below 15° C. Next, to initiate the second reaction step, concentrated ammonium hydroxide is added to a final concentration of 0.67 M. The reaction is allowed to stir at 45° C. for 2 hours. The reaction is filtered by vacuum with a GD120 filter and Buchner funnel, then treated with MB10 resin (10% w/v) until the electrical conductivity is below a threshold of 100 μS/cm. Resin is removed by vacuum filtration using a glass-fritted funnel and the filtrate is frozen, then lyophilized to dryness. The lyophilized product mixture is then solubilized in minimal ultra-pure H₂O after which a volume of 200 proof food-grade ethanol is added to create a 60% ethanol solution. The solution is then separated by centrifugation (4700 RPM, 15 min, −10° C.). The supernatant is carried forward while the pellet is once again solubilize din minimal ultra-pure H₂O after which a volume of 200 proof food-grade ethanol is added to create a 60% ethanol solution. The solution is then separated by centrifugation (4700 RPM, 15 min, −10° C.). The cumulative supernatant volume is reduced by rotary evaporation, then lyophilized to yield a fluffy, white, crystalline solid.

When analyzed the depolymerized product showed a composition wherein about 87.5% of the mass comprises glucose and about 4.5% of the mass comprises arabinose, as measured by hydrolytic monosaccharide compositional analysis. The glycosidic linkage composition comprises, approximately, the amount set forth in Table B, for CLX 115Cu. The oligosaccharide composition comprises, approximately, the values set forth in Table J (CLX115Cu), as measured by oligosaccharide analysis. The molecular weight distribution of the composition comprises, approximately, the values set forth in Table K (CLX115Cu), as measured by refractive index detection (RID) (see also FIG. 7B).

Therefore, it can be concluded that the optimized conditions can produce a desirable distribution of oligosaccharide molecular weights and yields.

### Example 7. Optimized Conditions for Iron-Based Fenton Depolymerization of Beta-Glucan

The use of iron-based and copper-based Fenton depolymerization has been demonstrated in several previous publications (WO2021097138A1, WO2018236917A1, WO2020247389A1). Here, we show a set of optimized parameters for the iron-based Fenton depolymerization of beta-glucan, particularly from cereals.

A solution containing 7% hydrogen peroxide with 43.4 mM, pH 5.5 ammonium acetate buffer is heated to 55° C. Beta Glucan (or other) polysaccharides are stirred in gradually to a final concentration of 5%. To initiate the first reaction step, Iron (II) sulfate is added to a final concentration of 1.15 mM. The reaction is allowed to proceed for 2 hours at 55° C. then cooled to below 15° C. Next, to initiate the second reaction step, concentrated ammonium hydroxide is added to a final concentration of 0.39 M. The reaction is allowed to stir at 45° C. for 2 hours. The reaction is filtered by vacuum with a GD120 filter and Buchner funnel, then treated with MB10 resin (10% w/v) until the electrical conductivity is below a threshold of 100 μS/cm. Resin is removed by vacuum filtration using a glass-fritted funnel and the filtrate is frozen, then lyophilized to dryness. The lyophilized product mixture is then solubilized in minimal ultra-pure H₂O after which a volume of 200 proof food-grade ethanol is added to create a 60% ethanol solution. The solution is then separated by centrifugation (4700 RPM, 15 min, −10° C.). The supernatant is carried forward while the pellet is once again solubilize din minimal ultra-pure H₂O after which a volume of 200 proof food-grade ethanol is added to create a 60% ethanol solution. The solution is then separated by centrifugation (4700 RPM, 15 min, −10° C.). The cumulative supernatant volume is reduced by rotary evaporation, then lyophilized to yield a fluffy, white, crystalline solid.

When analyzed the depolymerized product showed a composition wherein about 95% of the mass comprises glucose and about 2% of the mass comprises arabinose, as measured by hydrolytic monosaccharide compositional analysis. The glycosidic linkage composition comprises, approximately, the amount set forth in Table B, for CLX 115. The composition comprises, approximately, the 1H-13C HSQC NMR correlations set forth in Table A for CLX115. The oligosaccharide composition comprises, approximately, the values set forth in Table G (CLX115), as measured by oligosaccharide analysis. The molecular weight distribution of the composition comprises, approximately, the values set forth in Table K (CLX115), as measured by refractive index detection (RID) (see also FIG. 7C). The composition has a dynamic viscosity of about 1.382 mPa*s at 100 mg/ml at 25° C.

Therefore, it can be concluded that the optimized conditions can produce a desirable distribution of oligosaccharide molecular weights and yields.

### Example 8. Comparison Between Copper-Based and Iron-Based Fenton Depolymerization of Beta-Glucan

The use of iron-based and copper-based Fenton depolymerization has been demonstrated in several previous publications (WO2021097138A1, WO2018236917A1, WO2020247389A1). However, a thorough optimization of copper-based Fenton depolymerization has never been demonstrated. Here, we show a comparison of copper-based Fenton depolymerization and iron-based Fenton depolymerization of beta-glucan, particularly from cereals.

Copper-based Fenton depolymerization was performed as described in Example 6. Iron-based Fenton depolymerization was performed as described in Example 7.

Conclusion Therefore, it can be concluded that both the copper-based and iron-based Fenton depolymerization of beta glucans can produce similar oligosaccharide profiles. However, due to the higher substrate loading capacity, lower metal catalyst concentration, and lower hydrogen peroxide concentration, the copper-based Fenton depolymerization is believed to be more economically viable than the iron-based Fenton depolymerization.

### Example 9: Capsule Composition

A capsule is prepared by filling about 0.75 g of beta-glucan oligosaccharide into a 000 gelatine capsule using a filing machine. The capsules are then closed. The beta-glucan oligosaccharide is in free flowing, powder form.

### Example 10: Nutritional Composition

Beta-glucan oligosaccharide is introduced into a rotary blender in a 4:1 mass ratio. An amount of 0.25 wt % of silicon dioxide is introduced into the blender and the mixture blended for 10 minutes. The mixture is then agglomerated in a fluidized bed and filled into 5 gram stick packs and the packs are sealed.

### Example 11: In Vivo Evaluation of Beta-Glucan Effect on Blood Glucose Spike During Oral Challenge with Maltose

It has been described that some long carbohydrate structures significantly promote blood glucose regulation in normal mice (Cao et al. 2016). An in vivo assay using mice was performed to evaluate the effect of CLX115-Cu in the sugar spike after a single administration of maltose alone as a control or mixed with equal amounts of CLX115-Cu.

CLX115-Cu was produced from isolated beta-glucan from the method described in Example 8. Maltose or maltose plus CLX115-Cu were administered to two groups of healthy C57BL/6 male mice (n=15 mice/group). Mice were receiving a single gavage dose of 200 mg/ml of maltose alone or mixed with equal amount of CLX115-Cu. After administration, blood glucose levels were measured via handheld glucometer at pre-dose (0 h), 0.25 h, 0.5 h, 1 h. 1.5 h and 2 h post dose.

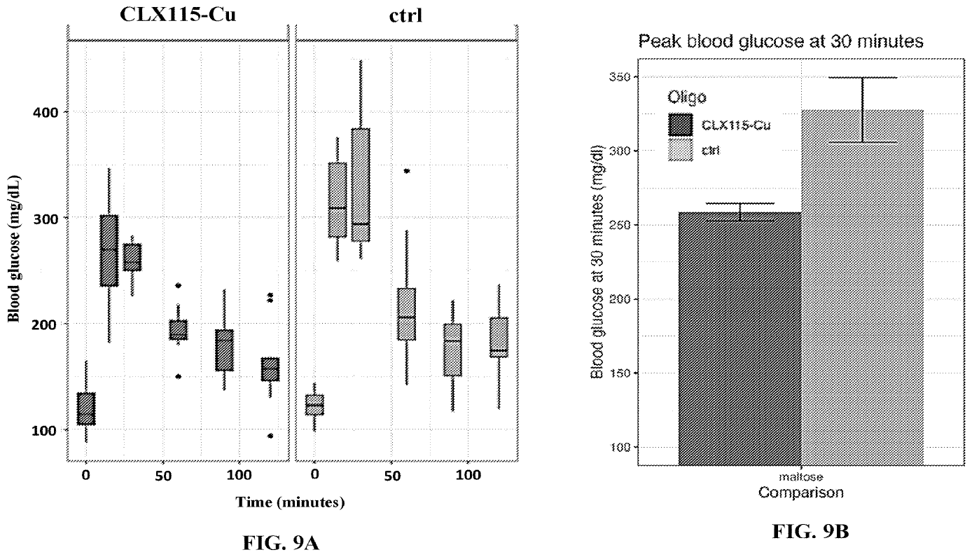

Blood glucose levels show expected trends during the experiment, with a peak of glucose between 30 and 90 minutes followed by a decline of glucose in blood between 90 and 120 minutes (FIG. 9A). CLX115-Cu significantly reduce the peak blood glucose relative to control (maltose alone) (two-way ANOVA). (FIG. 9B). This result indicates a possible interaction of CLX115-Cu with alpha-glucosidase enzymes, that reduces the amount of glucose in blood, as described in Example 2.

### Example 12: The Use of Copper-Based Fenton Depolymerization with Sodium Hydroxide as an Alternative Base

The use of an optimized copper-based Fenton depolymerization was demonstrated in previous examples in this application. Earlier applications demonstrated the use of sodium hydroxide as an alternative base (WO2021097138A1, WO2018236917A1, WO2020247389A1). However, the here described optimized copper-based Fenton depolymerization has not yet been described in tandem with the alternative sodium hydroxide base. Here, we show a set of optimized parameters and provide instructions for the copper-based Fenton depolymerization of beta-glucan with an alternative sodium hydroxide base.

A solution containing 4% hydrogen peroxide with 43.4 mM, pH 5.5 ammonium acetate buffer is heated to 55° C. Beta Glucan (or other) polysaccharides are stirred in gradually to a final concentration of 10%. To initiate the first reaction step, Copper (II) sulfate is added to a final concentration of 0.75 mM. The reaction is allowed to proceed for 2 hours at 55° C. then cooled to below 15° C. Next, to initiate the second reaction step, a 50% w/v sodium hydroxide solution is added to a final pH of 9; however, one can alternatively adjust the pH anywhere between the range of 8 and 12 and still achieve depolymerization to different degrees. The reaction is allowed to stir at 45° C. for 2 hours. The reaction is filtered by vacuum with a GD120 filter and Buchner funnel, then treated with MB10 resin (10% w/v) until the electrical conductivity is below a threshold of 100 μS/cm. Resin is removed by vacuum filtration using a glass-fritted funnel and the filtrate is frozen, then lyophilized to dryness. The lyophilized product mixture is then solubilized in minimal ultra-pure H₂O after which a volume of 200 proof food-grade ethanol is added to create a 60% ethanol solution. The solution is then separated by centrifugation (4700 RPM, 15 min, −10° C.). The supernatant is carried forward while the pellet is once again solubilized in minimal ultra-pure H₂O after which a volume of 200 proof food-grade ethanol is added to create a 60% ethanol solution. The solution is then separated by centrifugation (4700 RPM, 15 min, −10° C.). The cumulative supernatant volume is reduced by rotary evaporation, then lyophilized to yield a fluffy, white, crystalline solid.

The resulting depolymerization products of the copper-based Fenton depolymerization paired with the sodium hydroxide base are expected to be the same, or substantially similar to, the products described in Example 6.

### Example 13: The Use of Iron-Based Fenton Depolymerization with Sodium Hydroxide as an Alternative Base

The use of an optimized iron-based Fenton depolymerization was demonstrated in previous examples in this application. Earlier applications demonstrated the use of sodium hydroxide as an alternative base (WO2021097138A1, WO2018236917A1, WO2020247389A1). However, the here described optimized iron-based Fenton depolymerization has not yet been described in tandem with the alternative sodium hydroxide base. Here, we show a set of optimized parameters and provide instructions for the iron-based Fenton depolymerization of beta-glucan with an alternative sodium hydroxide base.

A solution containing 4% hydrogen peroxide with 43.4 mM, pH 5.5 ammonium acetate buffer is heated to 55° C. Beta Glucan (or other) polysaccharides are stirred in gradually to a final concentration of 10%. To initiate the first reaction step, Iron (II) sulfate is added to a final concentration of 1.15 mM. The reaction is allowed to proceed for 2 hours at 55° C. then cooled to below 15° C. Next, to initiate the second reaction step, a 50% w/v sodium hydroxide solution is added to a final pH of 9; however, one can alternatively adjust the pH anywhere between the range of 8 and 12 and still achieve depolymerization to different degrees. The reaction is allowed to stir at 45° C. for 2 hours. The reaction is filtered by vacuum with a GD120 filter and Buchner funnel, then treated with MB10 resin (10% w/v) until the electrical conductivity is below a threshold of 100 μS/cm. Resin is removed by vacuum filtration using a glass-fritted funnel and the filtrate is frozen, then lyophilized to dryness. The lyophilized product mixture is then solubilized in minimal ultra-pure H₂O after which a volume of 200 proof food-grade ethanol is added to create a 60% ethanol solution. The solution is then separated by centrifugation (4700 RPM, 15 min, −10° C.). The supernatant is carried forward while the pellet is once again solubilized in minimal ultra-pure H₂O after which a volume of 200 proof food-grade ethanol is added to create a 60% ethanol solution. The solution is then separated by centrifugation (4700 RPM, 15 min, −10° C.). The cumulative supernatant volume is reduced by rotary evaporation, then lyophilized to yield a fluffy, white, crystalline solid.

The resulting depolymerization products of the iron-based Fenton depolymerization paired with the sodium hydroxide base are expected to be the same, or substantially similar to, the products described in Example 7.

### REFERENCES

All references listed below, or anywhere else throughout this description, are hereby incorporated by reference herein in their entireties for all purposes:
Cao, Y., Zoe, S., Xu, H., Li, M., Tong, Z., Xu, M., Xu, X. Hypoglycemic activity of the Baker's yeast beta-glucan in obese/type 2 diabetic mice and the underlying mechanism. Mol Nutr Food Res. 2016, 60:2678-2690
Benalla, W., Bellahcen, S., Bnouham, M. Antidiabetic medicinal plants as source of alpha glucosidase inhibitors. Current Diabetes Rev. 2010, 6:247-254
Everard. A., Cani, P. D. Gut microbiota and GLP-1. Rev Endocr MEtab Disord, 2014, 15:189-196

### ASPECTS OF THE INVENTION

Various aspects are contemplated herein, several of which are set forth in the paragraphs below. It is explicitly contemplated that any aspect or portion thereof can be combined to form an aspect. In addition, it is explicitly contemplated that any aspect (e.g., Aspect A13) that references an aspect (e.g., Aspect A1) for which there are sub-aspects having the same top level number (e.g., Aspect A1a, A1b, A1c, and so forth) necessarily includes reference to those sub-aspects A1a, A1b, A1c, and so forth. In other words, if Aspect A13 refers to Aspect A1, and there are Aspects A1a and A1b present, then Aspect A13 refers to Aspects A1a or A1b. Furthermore, although the aspects below are subdivided into aspects A, B, C, D, and so forth, it is explicitly contemplated that aspects in each of subdivisions A, B, C, D, etc. can be combined in any manner. Moreover, the term “any preceding aspect” means any aspect that appears prior to the aspect that contains such phrase (in other words, the sentence “Aspect B13: The method of any one of aspects β1-B12, or any preceding aspect . . . ” means that any aspect prior to aspect B13 is referenced, including aspects β1-B12 and all of the “A” aspects). For example, it is contemplated that, optionally, any method or composition of any of the below aspects may be useful with or combined with any other aspect provided below. Further, for example, it is contemplated that any embodiment described elsewhere herein, including above this paragraph, may optionally be combined with any of the below listed aspects. In some instances in the aspects below, or elsewhere herein, two open ended ranges are disclosed to be combinable into a range. For example, “at least X” is disclosed to be combinable with “less than Y” to form a range, in which X and Y are numeric values. For the purposes of forming ranges herein, it is explicitly contemplated that “at least X” combined with “less than Y” forms a range of X-Y inclusive of value X and value Y.

Aspect A1. A method for treating glucose-related metabolic disorders in a subject, the method comprising enterally administering to the subject an effective amount of a beta-glucan oligosaccharide prior to and/or during the subject consuming a glucose source.

Aspect A2: The method of aspect A1, wherein the glucose-related metabolic disorder is diabetes.

Aspect A2a: The method of aspect A1 or A2, wherein the glucose-related metabolic disorder is Type 1 diabetes.

Aspect A2b: The method of any one of aspects A1-A2a, wherein the glucose-related metabolic disorder is Type 2 diabetes.

Aspect A2c: The method of any one of aspects A1-A2b, wherein the glucose-related metabolic disorder is gestational diabetes.

Aspect A3: The method of any one of aspects A1-A2c, wherein the glucose-related metabolic disorder is metabolic syndrome.

Aspect B1: A method for attenuating post-prandial glucose response in a subject, the method comprising enterally administering to the subject an effective amount of a beta-glucan oligosaccharide prior to and/or during the subject consuming a glucose source.

Aspect B2: The method according to aspect B1, or any preceding aspect, in which the subject is at risk of developing diabetes, is overweight and/or obese, is pregnant, and/or is diabetic.

Aspect C1: A method for reducing the risk of a prediabetic and/or obese subject from progressing to type 2 diabetes, the method comprising attenuating post-prandial glucose response in the subject by enterally administering to the subject an effective amount of a beta-glucan oligosaccharide prior to and/or during the subject consuming a glucose source.

Aspect D1: A method for lowering HbA1c levels in a subject, the method comprising attenuating the post-prandial glucose response in the subject over a period of at least 2 months by enterally administering to the subject an effective amount of a beta-glucan oligosaccharide prior to and/or during the subject consuming a glucose source.

Aspect E1: The method of any one of aspects A1-D1, wherein the effective amount of the beta-glucan oligosaccharide ranges from about 0.5 g to about 20 g. for example, from about 0.5 g to about 20 g, from about 0.5 g to about 15 g, from about 0.5 to about 10 g, from about 0.5 g to about 7.5 g, from about 0.75 g to about 20 g, from about 0.75 g to about 15 g, from about 0.75 g to about 15 g, from about 0.75 g to about 10 g, from about 0.75 g to about 7.5 g, from about 1 g to about 20 g, from about 1 g to about 15 g, from about 1 to about 10 g, or from about 1 g to about 7.5 g.

Aspect E2: The method of any one of aspects A1-D1, or any preceding aspect, wherein the effective amount of the beta-glucan oligosaccharide ranges from about 0.75 g to about 7.5 g, for example from about 0.75 g to about 20 g, from about 0.75 g to about 15 g, from about 0.75 g to about 15 g, from about 0.75 g to about 10 g, from about 0.75 g to about 7.5 g, from about 1 g to about 20 g, from about 1 g to about 15 g, from about 1 to about 10 g, or from about 1 g to about 7.5 g.

Aspect E3: The method of any one of aspects A1-E2, wherein the beta-glucan oligosaccharide is administered up to 2 hours prior to, up to 1.5 hours prior to, up to 1 hour prior to, or up to 30 minutes prior to, the subject consuming the glucose source.

Aspect E4: The method of any one of aspects A1-E3, wherein the beta-glucan oligosaccharide is administered up to 30 minutes prior to, up to 20 minutes prior to, or up to 15 minutes prior to, the subject consuming the glucose source.

Aspect E5: The method of any one of aspects A1-D1, or any preceding aspect, wherein the beta-glucan oligosaccharide is administered up to 30 minutes prior to, up to 20 minutes prior to, or up to 15 minutes prior to, the subject consuming the glucose source, and wherein the effective amount of the beta-glucan oligosaccharide ranges from about 0.5 g to about 20 g (e.g., from about 0.5 g to about 20 g, from about 0.5 g to about 15 g, from about 0.5 to about 10 g, from about 0.5 g to about 7.5 g. from about 0.75 g to about 20 g, from about 0.75 g to about 15 g, from about 0.75 g to about 15 g, from about 0.75 g to about 10 g, from about 0.75 g to about 7.5 g, from about 1 g to about 20 g, from about 1 g to about 15 g, from about 1 to about 10 g, or from about 1 g to about 7.5 g) or optionally ranges from about 0.75 g to about 7.5 g (e.g., from about 0.75 g to about 7.5 g. for example from about 0.75 g to about 20 g, from about 0.75 g to about 15 g, from about 0.75 g to about 15 g, from about 0.75 g to about 10 g, from about 0.75 g to about 7.5 g, from about 1 g to about 20 g, from about 1 g to about 15 g, from about 1 to about 10 g, or from about 1 g to about 7.5 g).

Aspect E6: The method of any one of aspects A1-E5, wherein the beta-glucan oligosaccharide inhibits salivary or pancreatic amylase.

Aspect E7: The method of any one of aspects A1-E6, wherein the beta-glucan oligosaccharide inhibits the SGLT1 glucose transporter.

Aspect E8: The method of any one of aspects A1-E7, wherein the beta-glucan oligosaccharide inhibits alpha-glucosidase.

Aspect E9: The method of any one of aspects A1-E8, wherein the beta-glucan oligosaccharide contains beta-1,3 and beta-1,4 linked glucose residues.

Aspect E9a: The method of any one of aspects A1-E9, wherein the beta-glucan oligosaccharide comprises CLX101, CLX102, CLX112. CLX115, CLX115Cu. CLX123, CLX125, or a combination thereof.

Aspect E9b: The method of any one of aspects A1-E9a, wherein the beta-glucan oligosaccharide comprises CLX112, CLX115, CLX115Cu, or a combination thereof.

Aspect E10: The method of any one of aspects A1-E9, wherein the beta-glucan oligosaccharide comprises β1,3 linked glucose residues: β1,4 linked glucose residues in a ratio of 1:1 to 1:5 (e.g., a ratio of 1:1 to 1:5, 1:2 to 1:5, 1:3 to 1:5, or 1:1 to 1:4).

Aspect E11: The method of any one of aspects A1-E10, wherein the beta-glucan oligosaccharide has a weight averaged molecular weight (Mw) of less than 10,000 Da (e.g., a Mw of between 100 Da and 10,000 Da, between 500 Da and 10,000 Da, or between 1,000 Da and 10,000 Da).

Aspect E12: The method of any one of aspects A1-E11, wherein the beta-glucan oligosaccharide has a weight averaged molecular weight (Mw) of less than 8,000 Da (e.g., a Mw of between 100 Da and 8,000 Da, between 500 Da and 8,000 Da, or between 1,000 Da and 8,000 Da).

Aspect E13: The method of any one of aspects A1-E12, wherein the beta-glucan oligosaccharide has a weight averaged molecular weight (Mw) of less than 7,500 Da (e.g., a Mw of between 100 Da and 7,500 Da, between 500 Da and 7,500 Da, or between 1,000 Da and 7.5000 Da).

Aspect E14: The method of any one of aspects A1-E13, wherein the beta-glucan oligosaccharide has a weight averaged molecular weight (Mw) of less than 5,000 Da (e.g., a Mw of between 100 Da and 5,000 Da, between 500 Da and 5,000 Da, or between 1,000 Da and 5.000 Da).

Aspect E15: The method of any one of aspects A1-E14, wherein the beta-glucan oligosaccharide contains 3 to 30 subunits (e.g., 3 to 30 subunits, 3 to 25 subunits, 5 to 30 subunits, 5 to 25 subunits, 10 to 30 subunits, or 10 to 25 subunits), wherein at least 50% of the subunits, at least 75% of the subunits, or 100% of the subunits are beta-1,3 glucose residues, beta-1,4 glucose residues, or a combination thereof.

Aspect E15a: The method of aspect E15, or any preceding aspect, wherein the beta-glucan oligosaccharide contains 3 to 30 subunits (e.g., 3 to 30 subunits, 3 to 25 subunits, 5 to 30 subunits, 5 to 25 subunits, 10 to 30 subunits, or 10 to 25 subunits), wherein each subunit is independently a beta-1,3 glucose residue or a beta-1,4 glucose residue.

Aspect E16: The method of any one of aspects A1-E15a, wherein the beta-glucan oligosaccharide contains 3 to 30 subunits (e.g., 3 to 30 subunits, 3 to 25 subunits, 5 to 30 subunits, 5 to 25 subunits, 10 to 30 subunits, or 10 to 25 subunits), wherein the beta-glucan oligosaccharide contains both beta-1,3 glucose residues and beta-1,4 glucose residues, and wherein at least 50% of the subunits, at least 75% of the subunits, or 100% of the subunits are beta-1,3 glucose residues or beta-1.4 glucose residues.

Aspect E16a: The method of any one of aspects A1-E16, wherein the beta-glucan oligosaccharide contains 3 to 30 subunits (e.g., 3 to 30 subunits, 3 to 25 subunits, 5 to 30 subunits, 5 to 25 subunits, 10 to 30 subunits, or 10 to 25 subunits), wherein the beta-glucan oligosaccharide contains both beta-1,3 glucose residues and beta-1,4 glucose residues, and wherein each subunit is independently a beta-1,3 glucose residue or a beta-1,4 glucose residue.

Aspect E17: The method of any one of aspects A1-E16a, wherein the beta-glucan oligosaccharide has a dynamic viscosity ranging from about 1 to about 10 mPa*s at 100 mg/ml at 25° C. (e.g., from about 1 to about 10 mPa*s at 100 mg/ml at 25° C., or from about 1 to about 7.5 mPa*s at 100 mg/ml at 25° C., or from about 1 to about 5 mPa*s at 100 mg/ml at 25° C.).

Aspect E18: The method of any one of aspects A1-E16a, or any preceding aspect, wherein the beta-glucan oligosaccharide has a dynamic viscosity ranging from about 1 to about 5 mPa*s at 100 mg/ml at 25° C. (e.g., from about 1 to about 5 mPa*s at 100 mg/ml at 25° C., or from about 1 to about 3 mPa*s at 100 mg/ml at 25° C., or from about 1 to about 1.5 mPa*s at 100 mg/ml at 25° C., or from about 1.3 to about 1.4 mPa*s at 100 mg/ml at 25° C.).

Aspect E19: The method of any one of aspects A1-E16a, or any preceding aspect, wherein the beta-glucan oligosaccharide has a dynamic viscosity ranging from about 1 to about 3 mPa*s at 100 mg/ml at 25° C. (e.g., from about 1 to about 3 mPa*s at 100 mg/ml at 25° C., or from about 1 to about 1.5 mPa*s at 100 mg/ml at 25° C., or from about 1.3 to about 1.4 mPa*s at 100 mg/ml at 25° C.).

Aspect E20: The method of any one of aspects A1-E16a, or any preceding aspect, wherein the beta-glucan oligosaccharide has a dynamic viscosity ranging from about 1 to about 1.5 mPa*s at 100 mg/ml at 25° C. (e.g., from about 1 to about 1.5 mPa*s at 100 mg/ml at 25° C., or from about 1.3 to about 1.4 mPa*s at 100 mg/ml at 25° C.).

Aspect E21: The method of any one of aspects A1-E16a, or any preceding aspect, wherein the beta-glucan oligosaccharide has a dynamic viscosity of about 1.3 to about 1.4 mPa*s at 100 mg/ml at 25° C.

Aspect E22: The method of any one of aspects A1-E21, wherein at least 70% of the mass (e.g., at least 70%, at least 75%, at least 85%, at least 95%, or optionally 100% of the mass) of beta-glucan oligosaccharide has molecular mass of less than 100 kDa (e.g., less than 100 kDa, less than 75 kDa, optionally greater than 0.1 kDa or 0.5 kDa).

Aspect E23: The method of any one of aspects A1-E21, or any preceding aspect, wherein at least 60% or the mass (e.g., at least 60%, at least 70%, at least 75%, at least 85%, at least 95%, or optionally 100% of the mass) of the beta-glucan oligosaccharide has molecular mass of less than 50 kDa (e.g., less than 50 kDa, less than 40 kDa, less than 30 kDa, less than 25 kDa, optionally greater than 0.1 kDa or 0.5 kDa).

Aspect E24: The method of any one of aspects A1-E21, or any preceding aspect, wherein at least 50% of the mass (e.g., at least 50%, at least 60%, at least 70%, at least 75%, at least 85%, at least 95%, or optionally 100% of the mass) of the beta-glucan oligosaccharide has molecular mass of less than 15 kDa (e.g., less than 15 kDa, less than 10 kDa, or less than 5 kDa, optionally greater than 0.1 kDa or 0.5 kDa).

Aspect E25: The method of any one of aspects A1-E21, or any preceding aspect, wherein at least 50% of the mass (e.g., at least 50%, at least 60%, at least 70%, at least 75%, at least 85%, at least 95%, or optionally 100% of the mass) of the beta-glucan oligosaccharide has molecular mass of less than 5 kDa (e.g., less than 5 kDa, less than 4 kDa, less than 2.5 kDa, or less than 1 kDa, optionally greater than 0.1 kDa).

Aspect E26: The method of any one of aspects A1-E21, or any preceding aspect, wherein at least 25% of the mass (e.g., at least 25%, at least 35%, at least 45%, at least 50%, at least 75%, at least 85%, at least 95%, or optionally 100% of the mass) of the beta-glucan oligosaccharide has a molecular mass of less than 1 kDa (e.g., less than 1 kDa or less than 0.75 kDa, optionally greater than 0.1 kDa).

Aspect E27: The method of any one of aspects A1-E26, wherein the beta-glucan oligosaccharide is generated by reacting polysaccharides in a reaction mixture with a Fenton's reagent, having a peroxide agent and metal ions, to provide treated polysaccharides; and cleaving the treated polysaccharides with a base to generate a mixture of polysaccharide cleavage products and/or oligosaccharides characteristic of the polysaccharides which mixture is the beta-glucan oligosaccharide.

Aspect E28: The method of aspect E27, or any preceding aspect, wherein, the Fenton's reagent comprises hydrogen peroxide, and one or more metal ions selected from the group consisting of transition metals Fe(II), Fe(III), Cu(I), Cu(II), Mn(II), Zn(II), Ni(II), and Co(II), alkaline earth metals Ca(II) and Mg(II), and the lanthanide Ce(IV).

Aspect E29: The method of aspect E27 or E28, or any preceding aspect, wherein the base is one or more bases selected from the group consisting of ammonium hydroxide, ammonium bicarbonate, ammonia, urea, sodium amide, dimethyl amine, trimethylamine, pyridine, and N,N-diisopropylethylamine, sodium hydroxide, calcium hydroxide, potassium hydroxide, barium hydroxide, lithium hydroxide.

Aspect E30: The method of any one of aspects E27-E29, or any preceding aspect, wherein the base is one or more bases selected from the group consisting of ammonium hydroxide and sodium hydroxide.

Aspect E31: The method of any one of aspects E27-E29, or any preceding aspect, wherein the base is a nitrogen-based cleavage reagent.

Aspect E32: The method of aspect E31, or any preceding aspect, wherein the nitrogen-based cleavage reagent is also a peroxide quenching reagent, and initiation of polysaccharide cleavage is commensurate, or substantially commensurate with initiation of peroxide-quenching.

Aspect E33: The method of aspect E31, or any preceding aspect, wherein, the nitrogen-based cleavage agent is not a peroxide-quenching agent, and the method further comprises initiation of peroxide quenching with an additional agent that is a peroxide-quenching agent.

Aspect E34: The method of any one of aspects E27-E33, or any preceding aspect, wherein the metal ion is a copper ion.

Aspect E35: The method of any one of aspects E27-E34, or any preceding aspect, wherein the metal ion is Cu(II).

Aspect E36: The method of aspect E35, or any preceding aspect, wherein Cu(II) is used in the reaction mixture at a concentration of about 0.25 mM to about 1.00 mM (e.g., about 0.25 mM to about 1.00 mM, about 0.25 mM to about 0.8 mM, about 0.5 mM to about 1 mM, or about 0.5 mM to about 0.8 mM).

Aspect E37: The method of aspect E35, or any preceding aspect, wherein Cu(II) is used in the reaction mixture at a concentration of about 0.7 mM to about 0.8 mM.

Aspect E38: The method of aspect E35, or any preceding aspect, wherein Cu(II) is used in the reaction mixture at a concentration of about 0.75 mM.

Aspect E39: The method of any one of aspects E27-E38, or any preceding aspect, wherein the Fenton's reagent comprises copper sulfate.

Aspect E40: The method of aspect E39, or any preceding aspect, wherein, copper sulfate is used in the reaction mixture at a concentration of about 0.25 mM to about 1.00 mM (e.g., about 0.25 mM to about 1.00 mM, about 0.25 mM to about 0.8 mM, about 0.5 mM to about 1 mM, or about 0.5 mM to about 0.8 mM).

Aspect E41: The method of aspect E39, or any preceding aspect, wherein copper sulfate is used in the reaction mixture at a concentration of about 0.7 mM to about 0.8 mM.

Aspect E42: The method of aspect E39, or any preceding aspect, wherein copper sulfate is used in the reaction mixture at a concentration of about 0.75 mM.

Aspect E43: The method of any one of aspects E27-E42, or any preceding aspect, wherein the hydrogen peroxide concentration is about 1% to about 7% (v/v) (e.g., about 1% to about 7%, about 1% to about 6%, about 1% to about 5%, about 1% to about 4.5%, about 3.5% to about 7%, or about 3.5% to 6% (v/v)).

Aspect E44: The method of any one of aspects E27-E42, or any preceding aspect, wherein the hydrogen peroxide concentration is about 3.5 to 4.5% (v/v).

Aspect E45: The method of any one of aspects E27-E42, or any preceding aspect, wherein, the hydrogen peroxide concentration is about 4.0 (v/v).

Aspect E46: The method of any one of aspects E27-E45, or any preceding aspect, wherein the base concentration is about 0.2 M to about 1 M (e.g., about 0.2 M to about 1.00 M, about 0.2 M to about 0.8 M, about 0.3 M to about 1 M, or about 0.3 M to about 0.8 M).

Aspect E47: The method of any one of aspects E27-E45, or any preceding aspect, wherein the base concentration is about 0.3 M to about 0.5 M.

Aspect E48: The method of any one of aspects E27-E45, or any preceding aspect, wherein the base concentration is about 0.4 M.

Aspect E49: The method of any one of aspects E27-E48, or any preceding aspect, wherein the base adjusts the pH of the mixture of polysaccharide cleavage products and/or oligosaccharides to a final pH of about 8 to 12.

Aspect E49a: The method of any one of aspects E27-E48, or any preceding aspect, wherein the base adjusts the pH of the mixture of polysaccharide cleavage products and/or oligosaccharides to a final pH of about 8.5 to about 11.

Aspect E50: The method of any one of aspects E27-E48, or any preceding aspect, wherein the base adjusts the pH of the mixture of polysaccharide cleavage products and/or oligosaccharides to a final pH of about 9.5 to about 10.5.

Aspect E51: The method of any one of aspects E27-E48, or any preceding aspect, wherein the base adjusts the pH of the mixture of polysaccharide cleavage products and/or oligosaccharides to a final pH of about 10.

Aspect E52: The method of any one of aspects E27-E51, or any preceding aspect, wherein the concentration of the polysaccharides reacted in the reaction mixture is between about 2% and about 20% (w/v) (e.g., between about 2% and 20%, between about 2% and 15%, between about 2% and 12%, between about 5% and 20%, between about 5% and 12%, or between about 8% and 20%).

Aspect E53: The method of any one of aspects E27-E51, or any preceding aspect, wherein the concentration of the polysaccharides reacted in the reaction mixture is between about 8% and about 12% (w/v).

Aspect E54: The method of any one of aspects E27-E51, or any preceding aspect, wherein the concentration of the polysaccharides reacted in the reaction mixture is about 10% (w/v).

Aspect E55: The method of any one of aspects E27-E54, or any preceding aspect, wherein at least one of the polysaccharides is derived from a grain.

Aspect E55a: The method of any one of aspects E27-E54, or any preceding aspect, wherein the polysaccharides are derived from a grain.

Aspect E56: The method of any one of aspects E27-E54, or any preceding aspect, wherein at least one of the polysaccharides is derived from oat or barley.

Aspect E56a: The method of any one of aspects E27-E54, or any preceding aspect, wherein the polysaccharides are derived from oat or barley.

Aspect E57: The method of any one of aspects E27-E56, or any preceding aspect, wherein at least one of the polysaccharides is a beta-glucan polysaccharide.

Aspect E57a: The method of any one of aspects E27-E57, or any preceding aspect, wherein the polysaccharides are beta-glucan polysaccharides.

Aspect E57b: The method of any one of aspects E27-E57a, or any preceding aspect, wherein the beta-glucan polysaccharide is CLX112-PS, CLX115-PS, or a combination thereof.

Aspect E58: The method of aspect E57 or E576, or any preceding aspect, wherein the beta-glucan polysaccharides have a weight average molecular weight of 500 kDa or more, optionally less than 10.000 kDa or less than 5,000 kDa.

Aspect F1: A beta-glucan oligosaccharide wherein at least 70% (e.g., at least 70%, at least 75%, at least 85%, at least 95%, or optionally 100%) of beta-glucan oligosaccharides by mass have a molecular mass of less than 100 kDa (e.g., less than 100 kDa, less than 75 kDa, or less than 50 kDa, optionally greater than 0.1 kDa or 0.5 kDa).

Aspect G1: A beta-glucan oligosaccharide wherein at least 60% (e.g., at least 60%, at least 70%, at least 75%, at least 85%, at least 95%, or optionally 100%) of beta-glucan oligosaccharides by mass have a molecular mass of less than 50 kDa (e.g., less than 50 kDa, less than 40 kDa, less than 30 kDa, less than 25 kDa, optionally greater than 0.1 kDa or 0.5 kDa).

Aspect H1: A beta-glucan oligosaccharide, wherein at least 50% (e.g., at least 50%, at least 60%, at least 70%, at least 75%, at least 85%, at least 95%, or optionally 100%) of beta-glucan oligosaccharides by mass have a molecular mass of less than 15 kDa (e.g., less than 15 kDa, less than 10 kDa, or less than 5 kDa, optionally greater than 0.1 kDa or 0.5 kDa).

Aspect I1: A beta-glucan oligosaccharide, wherein at least 50% (e.g., at least 50%, at least 60%, at least 70%, at least 75%, at least 85%, at least 95%, or optionally 100%) of beta-glucan oligosaccharides by mass have a molecular mass of less than 5 kDa (e.g., less than 5 kDa, less than 4 kDa, less than 2.5 kDa, or less than 1 kDa, optionally greater than 0.1 kDa).

Aspect J1: A beta-glucan oligosaccharide, wherein at least 25% (e.g., at least 25%, at least 35%, at least 45%, at least 50%, at least 75%, at least 85%, at least 95%, or optionally 100%) of beta-glucan oligosaccharides by mass have a molecular mass of less than 1 kDa (e.g., less than 1 kDa or less than 0.75 kDa, optionally greater than 0.1 kDa).

Aspect J1a: The beta-glucan oligosaccharide of any one of aspects F1-J1, or any preceding aspect, wherein the beta-glucan oligosaccharide comprises CLX101, CLX102, CLX112. CLX115, CLX115Cu, CLX123, CLX125, or a combination thereof.

Aspect J1b: The beta-glucan oligosaccharide of any one of aspects F1-J1a, wherein the beta-glucan oligosaccharide comprises CLX112, CLX115, CLX115Cu, or a combination thereof.

Aspect K1: The beta-glucan oligosaccharide of any one of aspects F1-J1, or any preceding aspect, wherein the beta-glucan oligosaccharide is generated by reacting polysaccharides in a reaction mixture with a Fenton's reagent, having a peroxide agent and metal ions, to provide treated polysaccharides; and cleaving the treated polysaccharides with a base to generate a mixture of polysaccharide cleavage products and/or oligosaccharides characteristic of the polysaccharides which mixture is the beta-glucan oligosaccharide.

Aspect K2: The beta-glucan oligosaccharide of aspect K1, or any preceding aspect, wherein the Fenton's reagent comprises hydrogen peroxide, and one or more metals selected from the group consisting of transition metals Fe(II), Fe(III), Cu(I), Cu(II), Mn(II). Zn(II), Ni(II), and Co(II), alkaline earth metals Ca(II) and Mg(II), and the lanthanide Ce(IV).

Aspect K3: The beta-glucan oligosaccharide of aspects K1 or K2, or any preceding aspect, wherein the base is one or more bases selected from the group consisting of ammonium hydroxide, ammonium bicarbonate, ammonia, urea, sodium amide, dimethyl amine, trimethylamine, pyridine, and N,N-diisopropylethylamine, sodium hydroxide, calcium hydroxide, potassium hydroxide, barium hydroxide, lithium hydroxide.

Aspect K4: The beta-glucan oligosaccharide of any one of aspects K1-K3, or any preceding aspect, wherein the base is one or more bases selected from the group consisting of ammonium hydroxide and sodium hydroxide.

Aspect K5: The beta-glucan oligosaccharide of any one of aspects K1-K4, or any preceding aspect, wherein the base is a nitrogen-based cleavage reagent.

Aspect K6: The beta-glucan oligosaccharide of aspect K5, or any preceding aspect, wherein the nitrogen-based cleavage reagent is also a peroxide quenching reagent, and initiation of polysaccharide cleavage is commensurate, or substantially commensurate with initiation of peroxide-quenching.

Aspect K7: The beta-glucan oligosaccharide of aspect K5, or any preceding aspect, wherein the nitrogen-based cleavage agent is not a peroxide-quenching agent, and the method further comprises initiation of peroxide quenching with an additional agent that is a peroxide-quenching agent.

Aspect K8: The beta-glucan oligosaccharide of any one of aspects K1-K7, or any preceding aspect, wherein the one or more metals are copper ions.

Aspect K9: The beta-glucan oligosaccharide of any one of aspects K1-K8, or any preceding aspect, wherein the one or more metals is Cu(II).

Aspect K10: The beta-glucan oligosaccharide of aspect K9, or any preceding aspect, wherein the Cu(II) is used at a concentration of about 0.25 mM to about 1.00 mM (e.g., about 0.25 mM to about 1.00 mM, about 0.25 mM to about 0.8 mM, about 0.5 mM to about 1 mM, or about 0.5 mM to about 0.8 mM).

Aspect K11: The beta-glucan oligosaccharide of aspect K9, or any preceding aspect, wherein the Cu(II) is used at a concentration of about 0.7 to about 0.8 mM.

Aspect K12: The beta-glucan oligosaccharide of aspect K9, or any preceding aspect, wherein the Cu(II) is used at a concentration of about 0.75 mM.

Aspect K13: The beta-glucan oligosaccharide of any one of aspects K1-K12, or any preceding aspect, wherein the hydrogen peroxide concentration is about 1% to about 7% (v/v) (e.g., about 1% to about 7%, about 1% to about 6%, about 1% to about 5%, about 1% to about 4.5%, about 3.5% to about 7%, or about 3.5% to 6% (v/v)).

Aspect K14: The beta-glucan oligosaccharide of any one of aspects K1-K12, or any preceding aspect, wherein the hydrogen peroxide concentration is about 3.5 to 4.5% (v/v).

Aspect K15: The beta-glucan oligosaccharide of any one of aspects K1-K12, or any preceding aspect, wherein the hydrogen peroxide concentration is about 4.0 (v/v).

Aspect K16: The beta-glucan oligosaccharide of any one of aspects K1-K15, or any preceding aspect, wherein the base concentration is about 0.2 M to about 1 M (e.g., about 0.2 M to about 1.00 M, about 0.2 M to about 0.8 M, about 0.3 M to about 1 M, or about 0.3 M to about 0.8 M).

Aspect K17: The beta-glucan oligosaccharide of any one of aspects K1-K15, or any preceding aspect, wherein the base concentration is about 0.3 M to about 0.5M.

Aspect K18: The beta-glucan oligosaccharide of any one of aspects K1-K15, or any preceding aspect, wherein the base concentration is about 0.4 M.

Aspect K19: The beta-glucan oligosaccharide any one of aspects K1-K18, or any preceding aspect, wherein the concentration of the polysaccharides reacted in the reaction mixture is between about 2% and about 20% (w/v) (e.g., between about 2% and 20%, between about 2% and 15%, between about 2% and 12%, between about 5% and 20%, between about 5% and 12%, or between about 8% and 20%).

Aspect K20: The beta-glucan oligosaccharide any one of aspects K1-K18, or any preceding aspect, wherein the concentration of the polysaccharides reacted in the reaction mixture is between about 8% and about 12% (w/v).

Aspect K21: The beta-glucan oligosaccharide any one of aspects K1-K18, or any preceding aspect, wherein the concentration of the polysaccharides reacted in the reaction mixture is about 10% (w/v).

Aspect K22: The beta-glucan oligosaccharide of any one of aspects K1-K21, or any preceding aspect, wherein at least one of the polysaccharides is derived from a grain.

Aspect K22a: The beta-glucan oligosaccharide of any one of aspects K1-K21, or any preceding aspect, wherein the polysaccharides are derived from a grain.

Aspect K23: The beta-glucan oligosaccharide of any one of aspects K1-K21, or any preceding aspect, wherein at least one of the polysaccharides is derived from oat or barley.

Aspect K23a: The beta-glucan oligosaccharide of any one of aspects K1-K21, or any preceding aspect, wherein the polysaccharides are derived from oat or barley.

Aspect K24: The beta-glucan oligosaccharide of any one of aspects K1-K21, or any preceding aspect, wherein at least one of the polysaccharides is a beta-glucan polysaccharide.

Aspect K24a: The beta-glucan oligosaccharide of any one of aspects K1-K21, or any preceding aspect, wherein at least one of the polysaccharides is a CLX112-PS or a CLX115-PS.

Aspect K24b: The beta-glucan oligosaccharide of any one of aspects K1-K21, or any preceding aspect, wherein the polysaccharides are beta-glucan polysaccharides.

Aspect K24c: The beta-glucan oligosaccharide of any one of aspects K1-K21, or any preceding aspect, wherein the polysaccharides are CLX112-PS, CLX115-PS, or a combination thereof.

Aspect K25: The beta-glucan oligosaccharide of aspect K24 or K24a, or any preceding aspect, wherein the beta-glucan polysaccharides have a weight average molecular weight of 500 kDa or more, optionally less than 10,000 kDa or less than 5,000 kDa.

Aspect L1: Use of the beta-glucan oligosaccharide of any one of aspects F1-K25, or any preceding aspect, for treating a glucose-based metabolic disorder.

Aspect L2: Use of the beta-glucan oligosaccharide of any one of aspects F1-K25, or any preceding aspect, for treating diabetes or metabolic syndrome.

Aspect L2a: The use of aspect L2, or any preceding aspect, for treating Type 2 diabetes.

Aspect L2b: The use of aspect L2 or L2a, or any preceding aspect, for treating Type 1 diabetes.

Aspect L3: Use of the beta-glucan oligosaccharide of any one of aspects F1-K25, or any preceding aspect, for attenuating post-prandial glucose response in a subject.

Aspect L3a: The use of aspect L3, or any preceding aspect, in which the subject is at risk of developing diabetes, is overweight and/or obese, is pregnant, and/or is diabetic.

Aspect L4: Use of the beta-glucan oligosaccharide of any one of aspects F1-K25, or any preceding aspect, for reducing the risk of a prediabetic and/or obese subject from progressing to Type 2 diabetes.

Aspect L5: Use of the beta-glucan oligosaccharide of any one of aspects F1-K25, or any preceding aspect, for lowering HbA1c levels in a subject.

Aspect L6: Use of the beta-glucan oligosaccharide of any one of aspects F1-K25, or any preceding aspect, for preparation of a medicament for attenuating post-prandial glucose response in a subject.

Aspect L6a: Use of aspect L6, or any preceding aspect, in which the subject is at risk of developing diabetes, is overweight and/or obese, is pregnant, and/or is diabetic.

Aspect L7: Use of the beta-glucan oligosaccharide of any one of aspects F1-K25, or any preceding aspect, for preparation of a medicament for treating a glucose-based metabolic disorder.

Aspect L8: The use of aspect L7, or any preceding aspect, for treating diabetes.

Aspect L8a: The use of aspect L8, or any preceding aspect, for treating Type 1 diabetes.

Aspect L8b: The use of aspect L8 or L8a, or any preceding aspect, for treating Type 2 diabetes.

Aspect L9: The use of aspect L7, or any preceding aspect, for treating metabolic syndrome.

Aspect L10: Use of the beta-glucan oligosaccharide of any one of aspects F1-K25, or any preceding aspect, for preparation of a medicament for reducing the risk of a prediabetic and/or obese subject from progressing to type 2 diabetes.

Aspect L11: Use of the beta-glucan oligosaccharide of any one of aspects F1-K25, or any preceding aspect, for preparation of a medicament for lowering HbA1c levels in a subject.

Aspect M1: A pharmaceutical composition comprising the beta-glucan oligosaccharide of any one of aspects F1-K25, or any preceding aspect, and a pharmaceutically acceptable carrier.

Aspect N1: A dietary supplement comprising the beta-glucan oligosaccharide of any one of aspects F1-K25, or any preceding aspect, and a food-grade carrier.

### STATEMENTS REGARDING INCORPORATION BY REFERENCE AND VARIATIONS

All references throughout this application, for example patent documents including issued or granted patents or equivalents; patent application publications; and non-patent literature documents or other source material; are hereby incorporated by reference herein in their entireties, as though individually incorporated by reference, to the extent each reference is at least partially not inconsistent with the disclosure in this application (for example, a reference that is partially inconsistent is incorporated by reference except for the partially inconsistent portion of the reference). All patents and publications mentioned in the specification are indicative of the levels of skill of those skilled in the art to which the invention pertains. References cited herein are incorporated by reference herein in their entirety to indicate the state of the art, in some cases as of their filing date, and it is intended that this information can be employed herein, if needed, to exclude (e.g., to disclaim) specific embodiments that are in the prior art. For example, when a compound is claimed, it should be understood that compounds known in the prior art, including certain compounds disclosed in the references disclosed herein (particularly in referenced patent documents), are not intended to be included in the claim.

The terms and expressions which have been employed herein are used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments, exemplary embodiments and optional features, modification and variation of the concepts herein disclosed may be resorted to by those skilled in the art, and that such modifications and variations are considered to be within the scope of this invention as defined by the appended claims. The specific embodiments provided herein are examples of useful embodiments of the present invention and it will be apparent to one skilled in the art that the present invention may be carried out using a large number of variations of the devices, device components, methods steps set forth in the present description. As will be obvious to one of skill in the art, methods and devices useful for the present methods can include a large number of optional composition and processing elements and steps.

As used herein and in the appended claims, the singular forms “a”, “an”, and “the” include plural reference unless the context clearly dictates otherwise. Thus, for example, reference to “a cell” includes a plurality of such cells and equivalents thereof known to those skilled in the art. As well, the terms “a” (or “an”), “one or more” and “at least one” can be used interchangeably herein. It is also to be noted that the terms “comprising”. “including”, and “having” can be used interchangeably. The expression “of any of claims XX-YY” (wherein XX and YY refer to claim numbers) is intended to provide a multiple dependent claim in the alternative form, and in some embodiments is interchangeable with the expression “as in any one of claims XX-YY.”

When a group of substituents is disclosed herein, it is understood that all individual members of that group and all subgroups, including any isomers, enantiomers, and diastereomers of the group members, are disclosed separately. When a Markush group or other grouping is used herein, all individual members of the group and all combinations and subcombinations possible of the group are intended to be individually included in the disclosure. When a compound is described herein such that a particular isomer, enantiomer or diastereomer of the compound is not specified, for example, in a formula or in a chemical name, that description is intended to include each isomers and enantiomer of the compound described individual or in any combination. Additionally, unless otherwise specified, all isotopic variants of compounds disclosed herein are intended to be encompassed by the disclosure. For example, it will be understood that any one or more hydrogens in a molecule disclosed can be replaced with deuterium or tritium. Isotopic variants of a molecule are generally useful as standards in assays for the molecule and in chemical and biological research related to the molecule or its use. Methods for making such isotopic variants are known in the art. Specific names of compounds are intended to be exemplary, as it is known that one of ordinary skill in the art can name the same compounds differently.

Certain molecules disclosed herein may contain one or more ionizable groups [groups from which a proton can be removed (e.g., —COOH) or added (e.g., amines) or which can be quaternized (e.g., amines)]. All possible ionic forms of such molecules and salts thereof are intended to be included individually in the disclosure herein. With regard to salts of the compounds herein, one of ordinary skill in the art can select from among a wide variety of available counterions those that are appropriate for preparation of salts of this invention for a given application. In specific applications, the selection of a given anion or cation for preparation of a salt may result in increased or decreased solubility of that salt.

Every device, system, formulation, combination of components, or method described or exemplified herein can be used to practice the invention, unless otherwise stated.

Whenever a range is given in the specification, for example, a temperature range, a time range, or a composition or concentration range, all intermediate ranges and subranges, as well as all individual values included in the ranges given are intended to be included in the disclosure. It will be understood that any subranges or individual values in a range or subrange that are included in the description herein can be excluded from the claims herein.

All patents and publications mentioned in the specification are indicative of the levels of skill of those skilled in the art to which the invention pertains. References cited herein are incorporated by reference herein in their entirety to indicate the state of the art as of their publication or filing date and it is intended that this information can be employed herein, if needed, to exclude specific embodiments that are in the prior art. For example, when composition of matter are claimed, it should be understood that compounds known and available in the art prior to Applicant's invention, including compounds for which an enabling disclosure is provided in the references cited herein, are not intended to be included in the composition of matter claims herein.

As used herein, “comprising” is synonymous with “including,” “containing,” or “characterized by,” and is inclusive or open-ended and does not exclude additional, unrecited elements or method steps. As used herein, “consisting of” excludes any element, step, or ingredient not specified in the claim element. As used herein, “consisting essentially of” does not exclude materials or steps that do not materially affect the basic and novel characteristics of the claim. For example, “consisting essentially of” in reference to a CLX or a pharmaceutical composition described herein does not exclude other compounds such as surfactants, flavoring agents, fillers, dyes, binders, buffering agents, preservatives, excipients, and carriers. In each instance herein any of the terms “comprising”, “consisting essentially of” and “consisting of” may be replaced with either of the other two terms. The invention illustratively described herein suitably may be practiced in the absence of any element or elements, limitation or limitations which is not specifically disclosed herein.

One of ordinary skill in the art will appreciate that starting materials, biological materials, reagents, synthetic methods, purification methods, analytical methods, assay methods, and biological methods other than those specifically exemplified can be employed in the practice of the invention without resort to undue experimentation. All art-known functional equivalents, of any such materials and methods are intended to be included in this invention. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

We claim:

1. A method for treating a glucose-related metabolic disorder in a subject, the method comprising enterally administering to the subject an effective amount of a beta-glucan oligosaccharide prior to and/or during the subject consuming a glucose source.

2. The method of claim 1, wherein the glucose-related metabolic disorder comprises diabetes, Type 1 diabetes, Type 2 diabetes, gestational diabetes, metabolic syndrome, or any combination thereof.

3. A method for attenuating post-prandial glucose response in a subject, the method comprising enterally administering to the subject an effective amount of a beta-glucan oligosaccharide prior to and/or during the subject consuming a glucose source.

4. The method of claim 4, wherein the subject is at risk of developing diabetes, is overweight, is obese, is pregnant, is diabetic, or any combination thereof.

5. A method for reducing the risk of a prediabetic and/or obese subject from progressing to Type 2 diabetes, the method comprising attenuating post-prandial glucose response in the subject by enterally administering to the subject an effective amount of a beta-glucan oligosaccharide prior to and/or during the subject consuming a glucose source.

6. A method for lowering HbA1c levels in a subject, the method comprising attenuating the post-prandial glucose response in the subject over a period of at least 2 months by enterally administering to the subject an effective amount of a beta-glucan oligosaccharide prior to and/or during the subject consuming a glucose source.

7. The method of any one of claims 1-6, wherein the effective amount of the beta-glucan oligosaccharide ranges from about 0.5 g to about 20 g.

8. The method of any one of claims 1-7, wherein the effective amount of the beta-glucan oligosaccharide ranges from about 0.75 g to about 15 g.

9. The method of any one of claims 1-8, wherein the beta-glucan oligosaccharide is administered up to 2 hours prior to the subject consuming the glucose source.

10. The method of any one of claims 1-9, wherein the beta-glucan oligosaccharide is administered up to 30 minutes prior to the subject consuming the glucose source.

11. The method of any one of claims 1-10, wherein the beta-glucan oligosaccharide is administered up to 30 minutes prior to the subject consuming the glucose source, and wherein the effective amount of the beta-glucan oligosaccharide ranges from about 0.5 g to about 20 g or optionally ranges from about 0.75 g to about 15 g.

12. The method of any one of claims 1-11, wherein the beta-glucan oligosaccharide inhibits salivary or pancreatic amylase.

13. The method of any one of claims 1-12, wherein the beta-glucan oligosaccharide inhibits the SGLT1 glucose transporter.

14. The method of any one of claims 1-13, wherein the beta-glucan oligosaccharide inhibits alpha-glucosidase.

15. The method of any one of claims 1-14, wherein the beta-glucan oligosaccharide contains beta-1,3 and beta-1,4 linked glucose residues.

16. The method of any one of claims 1-15, wherein the beta-glucan oligosaccharide comprises β1,3 linked glucose residues: $1,4 linked glucose residues in a ratio of 1:1 to 1:5.

17. The method of any one of claims 1-16, wherein the beta-glucan oligosaccharide has a weight averaged molecular weight (Mw) of less than 10,000 Da.

18. The method of any one of claims 1-17, wherein the beta-glucan oligosaccharide has a weight averaged molecular weight (Mw) of less than 5,000 Da.

19. The method of any one of claims 1-18, wherein the beta-glucan oligosaccharide comprises from 3 to 30 subunits, and wherein at least 50% of the subunits are beta-1,3 glucose residues, beta-1,4 glucose residues, or a combination thereof.

20. The method of any one of claims 1-19, wherein the beta-glucan oligosaccharide comprises from 3 to 30 subunits, wherein the beta-glucan oligosaccharide contains both beta-1,3 glucose residues and beta-1,4 glucose residues, and wherein each subunit is independently a beta-1,3 glucose residue or a beta-1,4 glucose residue.

21. The method of any one of claims 1-20, wherein the beta-glucan oligosaccharide has a dynamic viscosity ranging from about 1 to about 10 mPa*s at 100 mg/ml at 25° C.

22. The method of any one of claims 1-20, wherein the beta-glucan oligosaccharide has a dynamic viscosity ranging from about 1 to about 5 mPa*s at 100 mg/ml at 25° C.

23. The method of any one of claims 1-22, wherein at least 60% of the mass of the beta-glucan oligosaccharide has molecular mass of less than 50 kDa.

24. The method of any one of claims 1-23, wherein at least 50% of the mass of the beta-glucan oligosaccharide has molecular mass of less than 5 kDa.

25. The method of any one of claim 1-24 or 46-50, wherein the beta-glucan oligosaccharide is generated by reacting polysaccharides in a reaction mixture with a Fenton's reagent, having a peroxide agent and metal ions, to provide treated polysaccharides; and cleaving the treated polysaccharides with a base to generate a mixture of polysaccharide cleavage products and/or oligosaccharides characteristic of the polysaccharides which mixture is the beta-glucan oligosaccharide.

26. The method of claim 25, wherein the Fenton's reagent comprises hydrogen peroxide and one or more metal ions selected from the group consisting of transition metals Fe(II), Fe(III), Cu(I), Cu(II), Mn(II), Zn(II), Ni(II), and Co(II), alkaline earth metals Ca(II) and Mg(II), and the lanthanide Ce(IV).

27. The method of claim 25 or 26, wherein the base is one or more bases selected from the group consisting of ammonium hydroxide, ammonium bicarbonate, ammonia, urea, sodium amide, dimethyl amine, trimethylamine, pyridine, and N,N-diisopropylethylamine, sodium hydroxide, calcium hydroxide, potassium hydroxide, barium hydroxide, lithium hydroxide.

28. The method of any one of claims 25-27, wherein the base is one or more bases selected from the group consisting of ammonium hydroxide and sodium hydroxide.

29. The method of any one of claims 25-27, wherein the base is a nitrogen-based cleavage reagent.

30. The method of claim 29, wherein the nitrogen-based cleavage reagent is also a peroxide quenching reagent, and initiation of polysaccharide cleavage is commensurate, or substantially commensurate, with initiation of peroxide-quenching.

31. The method of claim 41, wherein, the nitrogen-based cleavage agent is not a peroxide-quenching agent, and the method further comprises initiation of peroxide quenching with an additional agent that is a peroxide-quenching agent.

32. The method of any one of claims 26-31, wherein the one or more metal ions comprises a copper ion.

33. The method of claim 32, wherein the copper ion is Cu(II).

34. The method of claim 33, wherein Cu(II) is used in the reaction mixture at a concentration of about 0.25 mM to about 1.00 mM.

35. The method of claim 33, wherein Cu(II) is used in the reaction mixture at a concentration of about 0.7 mM to about 0.8 mM.

36. The method of any one of claims 25-35, wherein the Fenton's reagent comprises copper sulfate.

37. The method of claim 36, wherein copper sulfate is used in the reaction mixture at a concentration of about 0.25 mM to about 1.00 mM.

38. The method of any one of claims 25-37, wherein the hydrogen peroxide concentration is about 1% to about 7% (v/v).

39. The method of any one of claims 25-38, wherein the base concentration is about 0.2 M to about 1 M.

40. The method of any one of claims 25-39, wherein the base adjusts the pH of the mixture of polysaccharide cleavage products and/or oligosaccharides to a final pH of about 8 to 12.

41. The method of any one of claims 25-40, wherein the concentration of the polysaccharides reacted in the reaction mixture is between about 2% and about 20% (w/v).

42. The method of any one of claims 25-41, wherein at least one of the polysaccharides reacted in the reaction mixture is derived from a grain.

43. The method of any one of claims 25-42, wherein at least one of the polysaccharides reacted in the reaction mixture is derived from oat or barley.

44. The method of any one of claims 25-43, wherein at least one of the polysaccharides reacted in the reaction mixture is a beta-glucan polysaccharide.

45. The method of claim 44, wherein the beta-glucan polysaccharide has a weight average molecular weight of 500 kDa or more.

46. A beta-glucan oligosaccharide, wherein at least 70% of beta-glucan oligosaccharides by mass have a molecular mass of less than 100 kDa.

47. A beta-glucan oligosaccharide, wherein at least 50% of beta-glucan oligosaccharides by mass have a molecular mass of less than 15 kDa.

48. The beta-glucan oligosaccharide of claim 46 or 47, wherein the beta-glucan oligosaccharide is derived from a cereal beta glucan.

49. The beta-glucan oligosaccharide of claim 48, wherein the beta-glucan oligosaccharide is derived from oat or barley.

50. The beta-glucan oligosaccharide of any one of claims 46-49, wherein the beta-glucan oligosaccharide comprises from 3 to 30 subunits, and wherein at least 50% of the subunits are beta-1.3 glucose residues, beta-1,4 glucose residues, or a combination thereof.

51. A pharmaceutical composition comprising the beta-glucan oligosaccharide of any one of claims 46-50 and a pharmaceutically acceptable carrier.

52. A dietary supplement comprising the beta-glucan oligosaccharide of any one of claims 46-50 and a food-grade carrier.
